(19) United States
(12) Patent Application Publication
(10) Pub. No.: US 2022/0298488 A1
(43) Pub. Date: Sep. 22, 2022

(54) Genetically Engineered Cells Sensitive for Clostridial Neurotoxins

(71) Applicant: SYNAPTIC RESEARCH, LLC, Haletehorpe, MD (US)

(72) Inventors: George OYLER, Wrexham (GB); Barry GERTZ, Wrexham (GB)

(21) Appl. No.: 17/596,140

(22) PCT Filed: Jun. 8, 2020
(86) PCT No.: PCT/IB2020/055377

§ 371 Date: Dec. 3, 2021

Related U.S. Application Data
(60) Provisional application No. 62/858,384, filed on Jun. 7, 2019.

(51) Int. Cl. C12N 5/09; C12N 15/85; C12N 5/079

(52) U.S. Cl. CPC C12N 5/0693; C12N 15/85; C12N 5/0618; C12N 2510/00

(57) ABSTRACT

A cell that has been genetically engineered to be highly sensitive to clostridial neurotoxin, for example, botulinum neurotoxin and tetanus neurotoxin, or modified or recombinant versions thereof. A method for making such a genetically-engineered cell and a method for using such a cell in assaying the activity of modified or recombinant clostridial neurotoxin.

### FIELD OF THE INVENTION

The present invention relates generally to a cell that has been genetically engineered to have increased sensitivity to clostridial neurotoxin, such as botulinum neurotoxin and tetanus neurotoxin. The invention also relates to a method for making such a cell and to a method for using such a cell in assaying the activity of polypeptides derived from such neurotoxins, such as modified and recombinant versions of such clostridial neurotoxins.

### BACKGROUND OF THE INVENTION

The anaerobic, gram-positive bacterium Clostridium botulinum produces various different types of neurotoxins, including botulinum neurotoxins (BoNTs) and tetanus neurotoxin (TeNT).

BoNTs are the most potent toxins known, with median lethal dose (LD₅₀) values for mice ranging from 0.5 to 5 ng/kg, depending on the serotype. BoNTs are adsorbed in the gastrointestinal tract and, after entering the general circulation, bind to the presynaptic membrane of cholinergic nerve terminals and prevent the release of the neurotransmitter acetylcholine.

BoNTs are well known for their ability to cause a flaccid muscle paralysis. Said muscle-relaxant properties have led to BoNTs being employed in a variety of medical and cosmetic procedures, including treatment of glabellar lines or hyperkinetic facial lines, headache, hemifacial spasm, hyperactivity of the bladder, hyperhidrosis, nasal labial lines, cervical dystonia, blepharospasm, and spasticity.

There are at present at least eight different classes of BoNT, namely: BoNT serotypes A, B, C, D, E, F, G, and H (known respectively as BoNT/A, BoNT/B, BoNT/C, BoNT/D, BoNT/E, BoNT/F, BoNT/G, and BoNT/H), all of which share similar structures and modes of action. Different BoNT serotypes can be distinguished based on inactivation by specific neutralising anti-sera, with such classification by serotype correlating with percentage sequence identity at the amino acid level. BoNT proteins of a given serotype are further divided into different subtypes on the basis of amino acid percentage sequence identity.

The different serotypes of BoNTs differ in affected animal species with regard to severity and duration of the paralysis caused. For example, BoNT/A is the most lethal of all known biological substances and, with regard to paralysis, is 500 times more potent in rats than BoNT/B. Further, the duration of paralysis after BoNT/A injection in mice is ten times longer than the duration following injection of BoNT/E.

In nature, clostridial neurotoxins are synthesised as a single-chain polypeptide that is modified post-translationally by a proteolytic cleavage event to form two polypeptide chains joined together by a disulfide bond. Cleavage occurs at a specific cleavage site, often referred to as the activation site, located between the cysteine residues that provide the inter-chain disulfide bond. It is this di-chain form that is the active form of the toxin. The two chains are termed the heavy chain (H-chain), which has a molecular mass of approximately 100 kDa, and the light chain (L-chain), which has a molecular mass of approximately 50 kDa. The H-chain comprises a C-terminal targeting component, known as the “targeting moiety” and an N-terminal translocation component, known as the “translocation domain”. The cleavage site is located between the L-chain and the translocation component, in an exposed loop region. Following binding of the targeting moiety to its target neuron and internalization of the bound toxin into the cell via an endosome, the translocation domain translocates the L-chain across the endosomal membrane and into the cytosol.

The L-chain comprises a protease component, known as the “protease domain”. It has a non-cytotoxic protease function and acts by proteolytically cleaving intracellular transport proteins known as SNARE proteins—see Gerald K (2002) “Cell and Molecular Biology” (4th edition) John Wiley & Sons, Inc. The acronym SNARE derives from the term Soluble NSF Attachment Receptor, where NSF means N-ethylmaleimide-Sensitive Factor. The protease domain has a zinc-dependent endopeptidase activity and exhibits a high substrate specificity for SNARE proteins.

Through their respective protease domains, the various different clostridial neurotoxins cleave different SNARE proteins. BoNT/B, BoNT/D, BoNT/F, BoNT/G, and TeNT cleave synaptobrevin, otherwise known as vesicle-associated membrane protein (VAMP). BoNT/A, BoNT/C, and BoNT/E cleave the synaptosomal-associated protein of 25 kDa (SNAP-25). BoNT/C cleaves syntaxin.

SNARE proteins are associated either with the membrane of a secretory vesicle or with a cell membrane and facilitate exocytosis of molecules by mediating the fusion of the secretory vesicle with the cell membrane, thus allowing for the contents of the vesicle to be expelled outside the cell. The cleavage of such SNARE proteins inhibits such exocytosis and thus inhibits the release of neurotransmitter from such neurons. As a result thereof, striated muscles are paralyzed and sweat glands cease their secretion.

Accordingly, once delivered to a desired target cell, the clostridial neurotoxins are capable of inhibiting cellular secretion from the target cell.

It is known in the art to modify clostridial neurotoxins to alter the properties thereof. Modifications can comprise amino acid modifications such as the addition, deletion, and/or substitution of amino acid(s) and/or chemical modifications such as addition of a phosphate or a carbohydrate or the formation of disulfide bonds. Modification can also involve the re-ordering of the components of the clostridial neurotoxin, for example, flanking the protease component with the translocation component and the targeting component.

It is also known in the art to produce recombinant clostridial neurotoxins that are either genetically identical to neurotoxin from Clostridia or that differ from wild-type clostridial neurotoxins in that they contain additional, fewer, or different amino acids and/or have the components placed in a different order from which they are placed in wild-type clostridial neurotoxins. These recombinant clostridial neurotoxins may also be chemically modified as described above.

The differences between the modified and recombinant clostridial neurotoxins and their wild-type counterparts, however, may affect the desired SNARE protein-cleaving property of the neurotoxin. As such, it may be important to determine whether such differences improve, reduce, or eliminate such activity.

Various conventional assays are available that allow the skilled artisan to confirm whether these modified or recombinant clostridial neurotoxins have the desired activity of cleaving the targeted SNARE protein. These assays involve testing for the presence of the products resulting from the cleavage of the SNARE protein. For example, following contacting of a cell with the modified or recombinant neurotoxin, the cell may be lysed and analyzed by SDS-PAGE to detect the presence of cleavage products. Alternatively, the cleavage products may be detected by contacting the cell lysate with antibodies.

While natural cells may be used in such assays, as such natural cells may have only limited sensitivity to clostridial neurotoxin, the assays typically require the use of a high concentration of such cells. In addition, it is desirable that such assays use a clonal stable cell line.

There is therefore a desire for a genetically-engineered cell that has increased sensitivity to clostridial neurotoxins for use in such assays.

### SUMMARY OF THE INVENTION

The present invention relates in part to a cell that has been genetically engineered to express or overexpress a receptor for clostridial neurotoxin, or a variant or fragment thereof. Such a receptor may be a protein receptor or a ganglioside. Preferably, the cell described herein (whether in the context of a cell per se described herein or any method or use which involves a cell described herein) comprises an exogenous nucleic acid that provides for expression or overexpression (preferably overexpression) of the receptor and/or ganglioside, wherein said exogenous nucleic acid is under the control of a constitutive and/or inducible promoter (preferably a constitutive promoter).

The invention also relates in part to a method of producing such a cell. The method comprises introducing into a cell a nucleic acid encoding: a clostridial neurotoxin receptor, or variant or fragment thereof that has the ability to bind clostridial neurotoxin; and/or an enzyme of the ganglioside synthesis pathway, or a variant or fragment thereof that has the catalytic activity of such enzyme.

The invention further relates in part to an assay for determining the activity of a modified or recombinant neurotoxin. The method comprises contacting the aforementioned cell with the modified or recombinant neurotoxin under conditions and for a period of time that would allow the protease domain of a wild-type clostridial neurotoxin to cleave an indicator protein in the cell and determining the presence of product resulting from the cleavage of such an indicator protein.

The invention embraces methods for testing/assessing the activity of a batch of clostridial neurotoxin for therapeutic/cosmetic use. Such methods advantageously find utility in toxin activity monitoring during storage, and tracking activity over time. A further advantage is the ability to determine optimal storage conditions (e.g. that do not degrade activity levels). The methods are particularly advantageous for characterizing the activity (e.g. cell binding/SNARE cleaving ability) of a recombinant clostridial neurotoxins.

An aspect of the invention provides an in vitro method for characterizing the activity of a clostridial neurotoxin (preferably BoNT) formulation or identifying a clostridial neurotoxin (preferably BoNT) formulation for therapeutic (and/or cosmetic) use, said method comprising:
a. providing a cell (e.g. genetically engineered cell) having an exogenous nucleic acid that provides for expression or overexpression of a receptor and/or ganglioside (preferably a receptor) having binding affinity for the clostridial neurotoxin, and an exogenous nucleic acid that provides for expression or overexpression of an indicator protein (preferably an indicator protein comprising a SNARE domain) cleavable by the clostridial neurotoxin; preferably wherein the cell does not express the receptor and/or ganglioside (preferably receptor) in the absence of said exogenous nucleic acid;
b. contacting said cell with the clostridial neurotoxin formulation;
c. comparing a level of cleavage of the indicator protein subsequent to contact with (e.g. administration of) the clostridial neurotoxin formulation with a level of cleavage pre-contact with the clostridial neurotoxin formulation; and
d. identifying (i) the clostridial neurotoxin formulation as being suitable for therapeutic (and/or cosmetic) use when the level of cleavage of the indicator protein subsequent to the contact is increased, or identifying (ii) the presence of activity when the level of cleavage of the indicator protein subsequent to the contact is increased; or
e. identifying (i) the clostridial neurotoxin formulation as being unsuitable for therapeutic (and/or cosmetic) use when the level of cleavage of the indicator protein subsequent to the contact is not increased, or identifying (ii) the absence of activity when the level of cleavage of the indicator protein subsequent to the contact is not increased.

Another aspect of the invention provides an in vitro method for characterising the activity of a clostridial neurotoxin (preferably BoNT) formulation or identifying a clostridial neurotoxin (preferably BoNT) formulation that is suitable for therapeutic (and/or cosmetic) use, said method comprising:
a. manipulating a cell (e.g. genetically engineering a cell) to incorporate an exogenous nucleic acid that provides for expression or overexpression of a receptor and/or ganglioside (preferably a receptor) having binding affinity for the clostridial neurotoxin, and an exogenous nucleic acid that provides for expression or overexpression of an indicator protein (preferably an indicator protein comprising a SNARE domain) cleavable by the clostridial neurotoxin; preferably wherein the cell does not express the receptor and/or ganglioside (preferably receptor) in the absence of said exogenous nucleic acid;
b. contacting said cell with the clostridial neurotoxin formulation;
c. comparing a level of cleavage of the indicator protein subsequent to contact with (e.g. administration of) the clostridial neurotoxin formulation with a level of cleavage pre-contact with the clostridial neurotoxin; and
d. identifying (i) the clostridial neurotoxin as being suitable for therapeutic (and/or cosmetic) use when the level of cleavage of the indicator protein subsequent to the contact is increased, or identifying (ii) the presence of activity when the level of cleavage of the indicator protein subsequent to the contact is increased; or
e. identifying (i) the clostridial neurotoxin as being unsuitable for therapeutic (and/or cosmetic) use when the level of cleavage of the indicator protein subsequent to the contact is not increased, or identifying (ii) the absence of activity when the level of cleavage of the indicator protein subsequent to the contact is not increased.

Another aspect of the invention provides an in vitro method for characterizing the activity of a clostridial neurotoxin (preferably BoNT) formulation or identifying a clostridial neurotoxin (preferably BoNT) formulation that is suitable for therapeutic (and/or cosmetic) use, said method comprising:
a. providing a cell (e.g. genetically engineered cell) having an exogenous nucleic acid that provides for expression or overexpression of a receptor and/or ganglioside (preferably a receptor) having binding affinity for the clostridial neurotoxin, and an exogenous nucleic acid that provides for expression or overexpression of an indicator protein (preferably an indicator protein comprising a SNARE domain) cleavable by the clostridial neurotoxin; preferably wherein the cell does not express the receptor and/or ganglioside (preferably receptor) in the absence of said exogenous nucleic acid;
b. contacting said cell with the clostridial neurotoxin formulation;
c. comparing a level of cleavage of the indicator protein subsequent to contact with (e.g. administration of) the clostridial neurotoxin formulation with a level of cleavage subsequent to contact with a control clostridial neurotoxin formulation; and
d. identifying (i) the clostridial neurotoxin formulation as being suitable for therapeutic (and/or cosmetic) use when the level of cleavage of the indicator protein subsequent to the contact is increased or equivalent to a level of cleavage subsequent to contact with a control clostridial neurotoxin formulation, or identifying (ii) the presence of activity when the level of cleavage of the indicator protein subsequent to the contact is increased or equivalent to a level of cleavage subsequent to contact with a control clostridial neurotoxin formulation; or
e. identifying (i) the clostridial neurotoxin as being unsuitable for therapeutic (and/or cosmetic) use when the level of cleavage of the indicator protein subsequent to the contact is not increased or equivalent to a level of cleavage subsequent to contact with a control clostridial neurotoxin formulation, or identifying (ii) the absence of activity when the level of cleavage of the indicator protein subsequent to the contact is not increased or not equivalent to a level of cleavage subsequent to contact with a control clostridial neurotoxin formulation.

The control clostridial neurotoxin formulation is a formulation of clostridial neurotoxin (e.g. batch of clostridial neurotoxin) that is known to have cleavage activity and/or is known to be suitable for therapeutic (and/or cosmetic) use (in other words, the control is a positive control). Preferably, the (test) clostridial neurotoxin formulation and control neurotoxin formulation are formulations of clostridial neurotoxin of the same type/ serotype, for example, where the (test) clostridial neurotoxin formulation is a formulation of BoNT/E, the control clostridial neurotoxin is preferably also a formulation of BoNT/E.

Another aspect of the invention provides a method for engineering a cell suitable for use in an assay for characterizing the activity of a clostridial neurotoxin (preferably BoNT) formulation or identifying a clostridial neurotoxin (preferably BoNT) formulation that is suitable for therapeutic (and/or cosmetic) use, the method comprising:
manipulating a cell to incorporate:
(i) an exogenous nucleic acid that provides for expression or overexpression of a receptor and/or ganglioside having binding affinity for the clostridial neurotoxin, preferably wherein the cell does not express the receptor and/or ganglioside (preferably receptor) in the absence of said exogenous nucleic acid; and
(ii) an exogenous nucleic acid that provides for expression or overexpression of an indicator protein (preferably an indicator protein comprising a SNARE domain) cleavable by the clostridial neurotoxin.

The following are optional embodiment for any aspect of the invention described herein.

In one embodiment, the cell does not normally express the receptor and/or ganglioside (preferably receptor, preferably wherein the receptor is SV2A). In other words, in a preferable embodiment, the cell does not express the receptor and/or ganglioside (preferably receptor, preferably wherein the receptor is SV2A) in the absence of said exogenous nucleic acid.

In one embodiment, the cell is of a cell type that is distinct from the natural target of the clostridial neurotoxin. In other words, in one embodiment, the cell is not a natural target of the clostridial neurotoxin. For example, the cell may not be a natural target of BoNT/A. Additionally or alternatively, the cell may not be a natural target of BoNT/E.

In one embodiment, the cell is not a neural cell (e.g. neuron).

Advantageously, by providing for expression or overexpression of a receptor and/or ganglioside having binding affinity for the clostridial neurotoxin, the methods and cells of the invention allow for reduced false-negative results (e.g. where low cleavage activity would be incorrectly detected as a result of low affinity of the clostridial neurotoxin for binding and translocating into the cell used in the assay, rather than low activity of the protease domain). Furthermore, the invention provides for a broader spectrum of cell types that may be used in such assays, reducing reliance on, for example, neural cells (e.g. naturally expressing sufficient levels of receptor/ ganglioside) which may be difficult to culture in vitro. These advantages are provided in the context of a cell-based assay (allowing for characterization of binding, translocation and protease activity) as opposed to a cell-free system (which characterizes protease activity only).

The term “when the level of cleavage of the indicator protein subsequent to the contact is not increased” means that there is substantially no increase in the level of cleavage. The term “substantially” as used herein in the context of the term “when the level of cleavage of the indicator protein subsequent to the contact is not increased” preferably means there is no statistically significant increase. Said increase (which is not substantial) may be an increase of less than 30%, 25%, 20%, 15%, 10%, 5% or 1%, preferably less than 20%. Said increase (which is not substantial) may be an increase of less than 5%, 2%, 1% or 0.5%, preferably less than 0.1%. More preferably, the term “when the level of cleavage of the indicator protein subsequent to the contact is not increased” as used herein means that the level of cleavage of the indicator protein subsequent to contact is not decreased at all (i.e. the increase in the level of cleavage is 0%).

A level of the receptor and/or ganglioside (preferably receptor) that is expressed in a cell described herein is preferably equal to or greater than a level of the receptor and/or ganglioside (preferably receptor) that is expressed in a natural target (e.g. neural cell) for the clostridial neurotoxin. For example, a level of the receptor and/or ganglioside (preferably receptor) that is expressed in a cell described herein may be ≥10%, ≥20%, ≥30%, ≥40%, ≥50%, ≥60%, ≥70%, ≥80%, ≥90%, or ≥100% relative to a level of the receptor and/or ganglioside (preferably receptor) that is expressed in a natural target (e.g. neural cell) for the clostridial neurotoxin.

A level of the receptor and/or ganglioside (preferably receptor) that is expressed in a cell described herein may preferably be greater than a level of the receptor and/or ganglioside (preferably receptor) that is expressed in a cell that lacks said exogenous nucleic acid. For example, a level of the receptor and/or ganglioside (preferably receptor) that is expressed in a cell described herein may be ≥10%, ≥20%, ≥30%, ≥40%, ≥50%, ≥60%, ≥70%, ≥80%, ≥90%, or ≥100% relative to a level of the receptor and/or ganglioside (preferably receptor) that is expressed in a cell (e.g. an otherwise equivalent cell) that lacks said exogenous nucleic acid.

In one embodiment, the term “overexpression” as used in the context of any aspect or embodiment described herein preferably means ≥10%, ≥20%, ≥30%, ≥40%, ≥50%, ≥60%, ≥70%, ≥80%, ≥90%, or ≥100% expression relative to a level of the receptor and/or ganglioside (preferably receptor) that is expressed in a natural target (e.g. neural cell) for the clostridial neurotoxin. In one embodiment, the term “overexpression” as used in the context of any aspect or embodiment described herein preferably means ≥10%, ≥20%, ≥30%, ≥40%, ≥50%, ≥60%, ≥70%, ≥80%, ≥90%, or ≥100% expression relative to a level of the receptor and/or ganglioside (preferably receptor) that is expressed in a cell (e.g. an otherwise equivalent cell) that lacks said exogenous nucleic acid.

In one embodiment, the clostridial neurotoxin may be BoNT/A (or a BoNT comprising a BoNT/A H_{CC} domain), and preferably the receptor may be SV2A, SV2B and/or SV2C (preferably SV2A).

Additionally or alternatively, the clostridial neurotoxin may be BoNT/B (or a BoNT comprising a BoNT/B H_{CC} domain), and preferably the receptor may be Syt-I and/or Syt-II.

Additionally or alternatively, the clostridial neurotoxin may be BoNT/E (or a BoNT comprising a BoNT/E H_{CC} domain), and preferably the receptor may be SV2A and/or SV2B (preferably SV2A).

Additionally or alternatively, the clostridial neurotoxin may be BoNT/G (or a BoNT comprising a BoNT/G H_{CC} domain), and preferably the receptor may be Syt-I and/or Syt-II.

For further information on suitable receptors/ gangliosides, see Binz and Rummel (Journal of Neurochemistry, Volume 109, Issue 6, June 2009, Pages 1584-1595), incorporated herein by reference.

### BRIEF DESCRIPTION OF THE DRAWINGS

FIG. 1A depicts a Western blot using anti-SNAP-25 antibody following treatment of N2a cells with BoNT/A at 0.1 nM, 1 nM, or 10 nM for 8 hours or at 1 nM, 0.1 nM, or 0.01 nM for 24 hours. The presence of a lower band indicates the presence of a cleavage product.

FIG. 1B depicts a Western blot using anti-SNAP-25 antibody following treatment of M17 cells with BoNT/A at 0.1 nM, 1 nM, or 10 nM for 8 hours or at 1 nM, 0.1 nM, or 0.01 nM for 24 hours. The presence of a lower band indicates the presence of a cleavage product.

FIG. 1C depicts a Western blot using anti-SNAP-25 antibody following treatment of IMR-32 cells with BoNT/A at 0.1 nM, 1 nM, or 10 nM for 8 hours or at 1 nM, 0.1 nM, or 0.01 nM for 24 hours. The presence of a lower band indicates the presence of a cleavage product.

FIG. 1D depicts a Western blot using anti-SNAP-25 antibody following treatment of NG108 cells with BoNT/A at 0.1 nM, 1 nM, or 10 nM for 8 hours or at 1 nM, 0.1 nM, or 0.01 nM for 24 hours. The presence of a lower band indicates the presence of a cleavage product.

FIG. 2A depicts fluorescent photomicrographs of NG108 cells 1 day after transfection with a plasmid containing the mScarlet-SNAP25-GeNluc construct.

FIG. 2B depicts fluorescent photomicrographs of M17 cells 1 day after transfection with a plasmid containing the mScarlet-SNAP25-GeNluc construct.

FIG. 3 depicts fluorescent photomicrographs of puromycin-resistant N108 cells stably transfected with a plasma containing the mScarlet-SNAP25-GeNluc construct.

FIG. 4 is a bar graph depicting average cell counts per HPF for cells that fluoresce green following treatment with 0, 0.1 nM, or 1 nM BoNT/A.

FIG. 5A depicts a scatter plot of flow cytometry data for NG108 cells stably transfected with the mScarlet-SNAP-25-GeNluc construct showing granularity/complexity on the x axis and cell size on the y axis.

FIG. 5B depicts a histogram of emission fluorescence intensity measured at 525 nm for NG108 cells stably transfected with the mScarlet-SNAP-25-GeNluc construct.

FIG. 5C depicts a histogram of emission fluorescence intensity measured at 585 nm for NG108 cells stably transfected with the mScarlet-SNAP-25-GeNluc construct.

FIG. 5D depicts a histogram of emission fluorescence intensity measured at 617 nm for NG108 cells stably transfected with the mScarlet-SNAP-25-GeNluc construct.

FIG. 5E depicts a histogram of emission fluorescence intensity measured at 665 nm for NG108 cells stably transfected with the mScarlet-SNAP-25-GeNluc construct.

FIG. 5F depicts a histogram of emission fluorescence intensity measured at 785 nm for NG108 cells stably transfected with the mScarlet-SNAP-25-GeNluc construct.

FIG. 5G depicts a scatter plot of flow cytometry data for NG108 cells stably transfected with the mScarlet-SNAP-25-GeNluc measured at 665 nm on the x axis and side-scatter (SS) on the y axis.

FIG. 6A depicts a scatter plot of flow cytometry data for M17 cells stably transfected with the mScarlet-SNAP-25-GeNluc construct showing granularity/complexity on the x axis and

cell size on the y axis. FIG. 6B depicts a histogram of emission fluorescence intensity measured at 525 nm for M17 cells stably transfected with the mScarlet-SNAP-25-GeNluc construct.

FIG. 6C depicts a histogram of emission fluorescence intensity measured at 585 nm for M17 cells stably transfected with the mScarlet-SNAP-25-GeNluc construct.

FIG. 6D depicts a histogram of emission fluorescence intensity measured at 617 nm for M17 cells stably transfected with the mScarlet-SNAP-25-GeNluc construct.

FIG. 6E depicts a histogram of emission fluorescence intensity measured at 665 nm for M17 cells stably transfected with the mScarlet-SNAP-25-GeNluc construct.

FIG. 6F depicts a histogram of emission fluorescence intensity measured at 785 nm for M17 cells stably transfected with the mScarlet-SNAP-25-GeNluc construct.

FIG. 6G depicts a scatter plot of flow cytometry data for M17 cells stably transfected with the mScarlet-SNAP-25-GeNluc measured at 665 nm on the x axis and side-scatter (SS) on the y axis.

FIG. 7A depicts a histogram of emission fluorescence intensity measured at 525 nm for control NG108 cells transfected with the mScarlet-SNAP25-GeNluc indicator construct.

FIG. 7B depicts a histogram of emission fluorescence intensity measured at 525 nm NG108 cells transfected with the mScarlet-SNAP-25-GeNluc indicator construct and treated with 0.1 nM BoNT/A.

FIG. 7C depicts a histogram of emission fluorescence intensity measured at 525 nm NG108 cells transfected with the mScarlet-SNAP-25-GeNluc indicator construct and treated with 1.0 nM BoNT/A.

FIG. 8A depicts a histogram of emission fluorescence intensity measured at 785 nm for control NG108 cells transfected with the mScarlet-SNAP25-GeNluc indicator construct.

FIG. 8B depicts a histogram of emission fluorescence intensity measured at 785 nm for NG108 cells transfected with the mScarlet-SNAP-25-GeNluc indicator construct and treated with 0.1 nM BoNT/A.

FIG. 8C depicts a histogram of emission fluorescence intensity measured at 785 nm for NG108 cells transfected with the mScarlet-SNAP-25-GeNluc indicator construct and treated with 1.0 nM BoNT/A.

FIG. 9 depicts a Western blot performed on NG108 cells transfected with the mScarlet-SNAP25-GeNluc indicator construct and treated with no toxin (control), 1 nM or 8 nM BoNT/A, or 0 (control), 1 nM, 10 nM, or 100 nM BoNT/E.

FIG. 10A depicts flow cytometry data for NG108 cells transfected with the mScarlet-SNAP25-GeNluc construct and not treated with toxin.

FIG. 10B depicts flow cytometry data for NG108 cells transfected with the mScarlet-SNAP25-GeNluc construct and treated with 10 nM BoNT/A for 72 hours.

FIG. 10C depicts flow cytometry data for NG108 cells transfected with the mScarlet-SNAP25-GeNluc construct and treated with 1 nM BoNT/A for 72 hours.

FIG. 10D depicts flow cytometry data for NG108 cells transfected with the mScarlet-SNAP25-GeNluc construct and treated with 0.1 nM BoNT/A for 72 hours.

FIG. 10E depicts flow cytometry data for NG108 cells transfected with the mScarlet-SNAP25-GeNluc construct and treated with 10 nM BoNT/E for 72 hours.

FIG. 10F depicts fluorescent photomicrographs of NG108 cells transfected with the mScarlet-SNAP25-GeNluc construct and not treated with toxin.

FIG. 10G depicts fluorescent photomicrographs of NG108 cells transfected with the mScarlet-SNAP25-GeNluc construct and treated with 10 nM BoNT/A for 72 hours.

FIG. 10H depicts fluorescent photomicrographs of NG108 cells transfected with the mScarlet-SNAP25-GeNluc construct and treated with 1 nM BoNT/A for 72 hours.

FIG. 10I depicts fluorescent photomicrographs of NG108 cells transfected with the mScarlet-SNAP25-GeNluc construct and treated with 0.1 nM BoNT/A for 72 hours.

FIG. 10J depicts fluorescent photomicrographs of NG108 cells transfected with the mScarlet-SNAP25-GeNluc construct and treated with 10 nM BoNT/E for 72 hours.

FIG. 11A depicts flow cytometry data for wild-type NG108 cells.

FIG. 11B depicts flow cytometry data for genetically-engineered NG108 cells that were selected for high expression of the indicator protein and sensitivity to BoNT/A at 1,000 pM and not further treated with BoNT/A.

FIG. 11C depicts flow cytometry data for genetically-engineered NG108 cells that were selected for high expression of the indicator protein and sensitivity to BoNT/A at 1,000 pM and that were treated with 100 pM BoNT/A for 48 hours.

FIG. 11D depicts flow cytometry data for genetically-engineered NG108 cells that were selected for high expression of the indicator protein and sensitivity to BoNT/A at 1,000 pM and that were treated with 100 pM BoNT/A for 96 hours.

FIG. 11E depicts flow cytometry data for genetically-engineered NG108 cells that were selected for high expression of the indicator protein but not for sensitivity to BoNT/A and not further treated with BoNT/A.

FIG. 11F depicts flow cytometry data for genetically-engineered NG108 cells that were selected for high expression of the indicator protein but not for sensitivity to BoNT/A and that were treated with 100 pM BoNT/A for 96 hours.

FIG. 12A depicts flow cytometry data for genetically-engineered NG108 cells that were selected for high expression of the indicator protein and sensitivity to BoNT/A at 100 pM and not further treated with BoNT/A.

FIG. 12B depicts flow cytometry data for genetically-engineered NG108 cells that were selected for high expression of the indicator protein and sensitivity to BoNT/A at 100 pM and that were treated with 100 pM BoNT/A for 96 hours.

FIG. 13A is a plot of the percent amount of indicator protein cleaved following the treatment of NG108 cells genetically engineered to express indicator protein and SV2A or SV2C with various concentrations of BoNT/A for various times.

FIG. 13B is a plot of the percent amount of indicator protein cleaved following the treatment of NG108 cells genetically engineered to express indicator protein and SV2A or SV2C with various concentrations of BoNT/E for various times.

### DETAILED DESCRIPTION OF THE INVENTION

It is to be understood that the present invention is not limited to the embodiments described herein. Indeed, numerous variations, changes, and substitutions will be apparent to those of skilled in the art without departing from the invention. It should be understood that various alternatives to the embodiments of the invention described herein may be employed in practicing the invention.

In describing the invention, where a range of values is provided with respect to an embodiment, it is understood that each intervening value is encompassed within the embodiment.

As used herein, a “variant” of a protein or polypeptide refers to a protein or polypeptide having an amino acid sequence that has at least 50%, 60%, 70%, 80%, 85%, 90%, 95%, 96%, 97%, 98%, 99%, 99.5%, or 99.9% sequence identity with the amino acid sequence of a reference protein or polypeptide.

“Sequence identity” as used herein refers to the identity between a reference amino acid or nucleotide sequence and a query amino acid or nucleotide sequence wherein the sequences are aligned so that the highest order match is obtained, and which can be calculated using published techniques or methods codified in computer programs such as, for example, BLASTP, BLASTN, FASTA (Altschul 1990, J. Mol. Biol. 215:403).

As used herein, a “fragment” of a protein or polypeptide refers to truncated forms of the protein or polypeptide or truncated forms of a variant of the protein or polypeptide.

The present invention relates in part to a cell that has been genetically engineered to have increased sensitivity to clostridial neurotoxin.

Clostridial neurotoxins are neurotoxins produced naturally by the bacteria Clostridium botulinum.

In certain embodiments of the present invention, the clostridial neurotoxin is a botulinum neurotoxin (BoNT) or a tetanus neurotoxin (TeNT). As used herein, the terms “clostridial neurotoxin”, “BoNT’, and “TeNT’, respectively, refer to wild-type clostridial neurotoxins, including those produced by strains other than Clostridium botulinum, as well as modified and recombinant clostridial neurotoxins.

Modified clostridial neurotoxins may contain one or more modifications as compared to wild-type clostridial neurotoxins, including amino acid modifications and/or chemical modifications. Amino acid modifications include deletions, substitutions, or additions of one or more amino acid residues. Chemical modifications include modifications made to one or more amino acid residues, such as the addition of a phosphate or a carbohydrate or the formation of disulfide bonds.

In certain embodiments, modifications may be made to alter the properties of the clostridial neurotoxin. The modifications to the clostridial neurotoxin may increase or decrease its biological activity.

The biological activity of clostridial neurotoxin encompasses at least three separate activities: the first activity is the “proteolytic activity” residing in the protease component of the neurotoxin and is responsible for hydrolysing the peptide bond of one or more SNARE proteins involved in the regulation of cellular membrane fusion. The second activity is the “translocation activity”, residing in the translocation component of the neurotoxin and is involved in the transport of the neurotoxin across the endosomal membrane and into the cytoplasm. The third activity is the “receptor binding activity”, residing at the targeting component of the neurotoxin and is involved in the binding of the neurotoxin to a receptor on a target cell.

In certain embodiments, the modification of a neurotoxin may involve truncating component(s) of the clostridial neurotoxin while still maintaining the activities of such component(s). For example, the neurotoxin may be modified to include only portion(s) of the protease component that are necessary for the proteolytic activity, only portion(s) of the translocation component that are necessary for the translocation activity, and/or only portion(s) of the targeting component that are necessary for the receptor binding activity.

Clostridial neurotoxin is initially produced as an inactive single-chain polypeptide and is placed in its active di-chain form following cleavage of the neurotoxin at its activation site. Such cleavage results in a di-chain protein with a heavy chain (H-chain) comprising the translocation and targeting components and a light chain (L-chain) comprising the protease component.

In certain embodiments, the biological activity of the clostridial neurotoxin is modified by modifying the activation site of the neurotoxin. The ability of the neurotoxin to be activated may thereby be increased, decreased, or remain the same. In certain embodiments, the biological activity of the clostridial neurotoxin is increased or triggered by modifying the activation site so that it is more readily cleaved, thus activating the neurotoxin. In embodiments wherein activation is only desired in certain environments or cells, the activation site may be modified so that it is only cleaved by proteases present in such environments or cells. In certain other environments, the biological activity of the neurotoxin is decreased or inactivated by modifying the activate site so that it is less readily cleaved.

In certain embodiments, the biological activity of the clostridial neurotoxin is modified by modifying the protease component of the neurotoxin. The proteolytic activity of the neurotoxin may thereby be increased, decreased, or remain the same. In certain embodiments, the protease component may be replaced with a protease component from a different clostridial neurotoxin or a variant or fragment thereof. For example, a BoNT/A may be modified by replacing its protease component with the protease component of BoNT/E.

In certain embodiments, the biological activity of the clostridial neurotoxin is modified by modifying the translocation component of the neurotoxin. The translocation activity of the neurotoxin may thereby be increased, decreased, or remain the same. In certain embodiments, the translocation component may be replaced with a translocation component from a different clostridial neurotoxin or a variant or fragment thereof. For example, a BoNT/A may be modified by replacing its translocation component with the translocation component of BoNT/E.

In certain embodiments, the biological activity of the clostridial neurotoxin is modified by modifying the targeting component of the neurotoxin. The targeting ability of the neurotoxin may thereby be increased, decreased, or remain the same. In certain embodiments, the targeting component may be replaced with a targeting component from a different clostridial neurotoxin or a variant or fragment thereof. For example, a BoNT/A may be modified by replacing its targeting component with the targeting component of BoNT/E. In certain other embodiments, the targeting component may be replaced with a non-clostridial polypeptide, for example, an antibody.

Also, modification can involve the re-ordering of the components of the clostridial neurotoxin, for example, flanking the protease component with the translocation component and the targeting component.

Recombinant clostridial neurotoxins are genetically produced. They may either be genetically identical to wild-type clostridial neurotoxin or differ from wild-type clostridial neurotoxins in that they contain additional, fewer, or different amino acids. For example, recombinant clostridial neurotoxins mirroring any of the aforementioned modified clostridial neurotoxins may be made. Recombinant clostridial neurotoxins may also have the components placed in a different order from which they are placed in wild-type clostridial neurotoxins. Recombinant clostridial neurotoxins may also be chemically modified as described above.

In certain embodiments, the modified or recombinant clostridial neurotoxin is a polypeptide having an amino acid sequence that has at least 50%, 60%, 70%, 80%, 90%, 95%, 97%, 98%, or 99% sequence identity with a wild-type clostridial neurotoxin, for example a BoNT of serotype A, B, C, D, E, F, G, or H, or a TeNT.

A series of programs based on a variety of algorithms is available to the skilled artisan for comparing different sequences. In this context, the algorithms of Needleman and Wunsch or Smith and Waterman give particularly reliable results. To carry out the sequence alignments and calculate the sequence identity values recited herein, the commercially available program DNASTAR Lasergene MegAlign version 7.1.0 based on the algorithm Clustal W was used over the entire sequence region with the following settings: Pairwise Alignment parameters: Gap Penalty: 10.00, Gap Length Penalty: 0.10, Protein weight matrix Gonnet 250, which, unless otherwise specified, shall always be used as standard settings for sequence alignments.

The BoNT/A serotype is divided into at least six sub-serotypes (also known as subtypes), BoNT/A1 to BoNT/A6, which share at least 84%, up to 98%, amino acid sequence identity. BoNT/A proteins within a given subtype share a higher amino acid percentage sequence identity.

Clostridial neurotoxins target neurons by binding to receptors. Receptors for clostridial neurotoxin include protein receptors and plasma membrane gangliosides.

Gangliosides are oligoglycosylceramides derived from lactosylceramide and containing a sialic acid residue such as N-acetylneuraminic acid (Neu5Ac), N-glycolyl-neuraminic acid (Neu5Gc), or 3-deoxy-D-glycero-D-galacto-nonulosonic acid (KDN). Gangliosides are present and concentrated on cell surfaces, with the two hydrocarbon chains of the ceramide moiety embedded in the plasma membrane and the oligosaccharides located on the extracellular surface, where they present points of recognition for extracellular molecules or surfaces of neighboring cells. Gangliosides also bind specifically to viruses and to bacterial toxins, such as clostridial neurotoxins.

Gangliosides are defined by a nomenclature system in which M, D, T and Q refer to mono-, di-, tri- and tetrasialogangliosides, respectively, and the numbers 1, 2, 3, etc. refer to the order of migration of the gangliosides on thin-layer chromatography. For example, the order of migration of monosialogangliosides is GM3>GM2>GM1. To indicate variations within the basic structures, further terms are added, e.g. GM1a, GD1b, etc. Glycosphingolipids having 0, 1, 2, and 3 sialic acid residues linked to the inner galactose unit are termed asialo- (or 0-), a-, b- and c-series gangliosides, respectively, while gangliosides having sialic acid residues linked to the inner N-galactosamine residue are classified as a-series gangliosides. Pathways for the biosynthesis of the 0-, a-, b- and c- series of gangliosides involve sequential activities of sialyltransferases and glycosyltransferases as illustrated, for example, in Ledeen et al., Trends in Biochemical Sciences, 40: 407-418 (2015). Further sialization of each of the series and in different positions in the carbohydrate chain can occur to give an increasingly complex and heterogeneous range of products, such as the a-series gangliosides with sialic acid residue(s) linked to the inner N-acetylgalactosamine residue. Gangliosides are transferred to the external leaflet of the plasma membrane by a transport system involving vesicle formation.

So far, nearly 200 gangliosides have been identified in vertebrate tissues. Common gangliosides include: GM1; GM2; GM3; GD1a; GD1b; GD2; GD3; GT1b; GT3; and GQ1.

Clostridial neurotoxins possess two independent binding regions in the H_{CC} domain for gangliosides and neuronal protein receptors. BoNT/A, BoNT/B, BoNT/E, BoNT/F and BoNT/G have a conserved ganglioside binding site in the H_{CC} domain composed of a “E(Q) . . . H(K) . . . SXWY . . . G” motif, whereas BoNT/C and BoNT/D display two independent ganglioside-binding sites. Lam et al., Progress in Biophysics and Molecular Biology, 117:225-231 (2015). Most BoNTs bind only to gangliosides that have a 2,3-linked N-acetylneuraminic acid residue (denoted Sia5) attached to Gal4 of the oligosaccharide core, whereas the corresponding ganglioside-binding pocket on TeNT can also bind to GM1a, a ganglioside lacking the Sia5 sugar residue. BoNT/D has been found to bind GM1a and GD1a. See Kroken et al., Journal of Biological Chemistry, 286:26828-26837 (2011). Combining the data derived from ganglioside-deficient mice and biochemical assays, BoNT/A, BoNT/E, BoNT/F and BoNT/G display a preference for the terminal NAcGal-Gal-NAcNeu moiety being present in GD1a and GT1b, whereas BoNT/B, BoNT/C, BoNT/D and TeNT require the disialyl motif found in GD1b, GT1b and GQ1b. Abundant complex polysialo-gangliosides such as GD1a, GD1b and GT1b thus appear essential to specifically accumulate all BoNT serotypes and TeNT on the surface of neuronal cells as the first step of intoxication. See Rummel, Andreas, “Double receptor anchorage of botulinum neurotoxins accounts for their exquisite neurospecificity,” Botulinum Neurotoxins, Springer Berlin Heidelberg (2012) 61-90.

In view of the above, in certain embodiments of the present invention, the cell is genetically engineered to express or overexpress a ganglioside. In particular embodiments, the cell is genetically engineered to express or overexpress GM1a, GD1a, GD1b, GT1b, and/or GQ1b. In certain embodiments, the cell has been engineered to express or overexpress GD1a, GD1b, and/or GT1b. In certain embodiments, the cell has been engineered to express or overexpress GD1b and/or GT1b.

Gangliosides are synthesized starting from ceramide. From ceramide, one pathway involves the addition of a glucose unit by glucosylceramide synthase to form glucosylceramide (GlcCer). β1,4-galactosyltransferase I (GalT-I) then catalyzes the addition of a galactose unit to GlcCer to form lactosylceramide (LacCer). From LacCer, GalNAc-transferase (GalNAcT) may add N-acetylgalactosamine to form GA1 or GM3 synthase may add a sialic acid to form GM3. From GM3, GD3 may be formed by the addition of a further sialic acid by GD3 synthase. GT3 may be formed from GD3 by the addition of yet further sialic acid by GT3 synthase. In a separate pathway, a galactose unit is added to LacCer by galactosylceramide synthase to form galactosylceramide (GalCer). A further carbohydrate group is then added by GM4 synthase to form GM4. GM3, GD3, and GT3 may then be modified to form more complex gangliosides of the “a”, “b”, or “c” series, respectively. Such reactions are catalyzed by GalNAcT, β1,3-galactosyltransferase II (GalT-II), α2,3-sialyltranferase IV (ST-IV), or α2,8-sialyltransferase V (ST-V). For example, from GD3, the “b” series gangliosides GD1b, GT1b, and GQ1b are formed.

The cells of the present invention may therefore be engineered to express or overexpress a desired ganglioside by being engineered to express or overexpress an enzyme of the biosynthesis pathway that leads to the ganglioside. For example, the cell may engineered (i.e., by transfection) to contain an exogenous nucleic acid encoding such an enzyme. Thus, in certain embodiments, the cell has been engineered to express or overexpress glucosylceramide synthase, GalT-I, GalNAcT, GM3 synthase, GD3 synthase, GT3 synthase, galactosylceramide synthase, GM4 synthase, GalT-II, ST-IV, and/or ST-V.

The skilled artisan would understand that variants or fragments of such enzymes that retain the desired catalytic activity thereof can also play a role in the synthesis of the gangliosides of interest. Thus, in certain embodiments, the cell has been genetically engineered to express or overexpress a variant or a fragment of an enzyme of the ganglioside synthesis pathway that retains the ability of that enzyme. For example, in certain embodiments, the cell has been genetically engineered to express or overexpress a variant or fragment of glucosylceramide synthase that has the ability to add glucose to ceramide, a variant or fragment of GalT-I that has the ability to add a galactose unit to GlcCer, a variant or fragment of GalNAcT that has the ability to add N-acetylgalactosamine to LacCer, a variant or fragment of GM3 synthase that has the ability to add a sialic acid to LacCer, a variant or fragment of GD3 synthase that has the ability to add a sialic acid to GM3, a variant or fragment of GT3 synthase that has the ability to add a sialic acid to GD3, a variant or fragment of galactosylceramide synthase that has the ability to add a galactose unit to LacCer, and/or a variant of GM4 synthase that has the ability to add a carbohydrate group to GalCer.

In certain embodiments, the variant is a protein that has an amino acid sequence that has at least 80%, 85%, 90%, 95%, 98%, or 99% sequence identity with that of an enzyme of the biosynthesis pathway that leads to the ganglioside and that retains the desired catalytic activity of such enzyme. In certain such embodiments, the variant is a protein that has an amino acid sequence that has at least 80%, 85%, 90%, 95%, 98%, or 99% sequence identity with glucosylceramide synthase, GalT-I, LacCer, GalNAcT, GD3 synthase, GT3 synthase, galactosylceramide, GM4 synthase, GalT-II, ST-IV, and/or ST-V and that retains the desired catalytic activity of that enzyme.

The fragment may, for example, have 50 amino acids or less, 40 amino acids or less, 30 amino acids or less, 20 amino acids or less, or 10 amino acids or less.

Assays are known in the art that can be used to determine which variants or fragments have the desired catalytic activity. For example, the skilled artisan would be aware of assays that may be used to determine whether a variant or fragment of GD3 synthase has the ability to add sialic acid to GM3.

The skilled artisan would understand that the aforementioned enzymes may also be encoded by nucleic acids that differ from the aforementioned exogenous nucleic acid by conservative substitutions, which are known in the art. The skilled artisan would also understand that variants of the enzymes may, for example, be encoded by nucleic acids that have at least 80%, 85%, 90%, 95%, 98%, or 99% sequence identity with the nucleic acid encoding the wild-type enzyme. Thus the invention also contemplates a cell that has been genetically engineered to contain an exogenous nucleic acid that has at least 80%, 85%, 90%, 95%, 98%, or 99% sequence identity with a nucleic acid that encodes one of the aforementioned enzymes and/or a nucleic acid that differs from the wild-type nucleic acid encoding such an enzyme by only conservative substitutions, wherein the encoded protein is the wild-type enzyme or a variant that retains the catalytic activity of the wild-type enzyme.

In certain embodiments, the cell is engineered to express or overexpress an enzyme that serves to catalyze what has been determined to be a rate-limiting step in the biosynthesis of a desired ganglioside, or a variant or fragment thereof that has the desired catalytic activity of such an enzyme. For example, GD3 synthase is an enzyme that catalyzes a rate-limiting step in the biosynthesis of “b” series gangliosides, specifically the addition of a sialic acid to GM3. Thus, in embodiments wherein the expression or overexpression of GD1b, GT1b, and/or GQ1b are desired, the cell is engineered to express or overexpress GD3 synthase or a variant or fragment thereof that has the ability to add sialic acid to GM3.

For example, the cell may be transfected with a nucleic acid encoding GD3 synthase or a nucleic acid, as described above, having at least 80%, 85%, 90%, 95%, 98%, or 99% sequence identity with such a nucleic acid, for example one that encodes a variant of GD3 synthase that retains the catalytic activity thereof or one that differs from wild-type GD3 synthase only in conservative substitutions.

The binding of certain clostridial neurotoxins to cells may also be reliant on binding to protein receptors. BoNT/A, BoNT/D, BoNT/E, BoNT/F, and TeNT bind to synaptic vesicle protein 2 (SV2) with BoNT/A capable of binding to all three isoforms thereof (SV2A, SV2B, and SV2C) and BoNT/E capable binding to only the SV2A and SV2B isoforms. BoNT/B and BoNT/G bind to both isoforms (I and II) of synaptotagmin. Synaptotagmin and SV2 are localized on synaptic vesicles and become exposed to extracellular space when the vesicles fuse with the presynaptic membrane. It is during this period that the clostridial neurotoxins bind to their protein receptors.

The cells of the present invention may therefore be engineered to express or overexpress a desired protein receptor, for example, an SV2 (e.g., SV2A, SV2B, and SV2C) or an synaptotagmin (e.g., synaptotagmin I and synaptotagmin II). For example, the cell may engineered (e.g., by transfection) to contain an exogenous nucleic acid encoding such a protein receptor.

The present invention also contemplates proteins that differ from such protein receptors but still retain the ability to bind clostridial neurotoxin. Such proteins may be a variant or fragment of such a protein receptor that retains the ability of the receptor to bind clostridial neurotoxin. Thus, in certain embodiments, the cell has been engineered to express or overexpress a variant or fragment of SV2 that binds BoNT/A, BoNT/D, BoNT/E, BoNT/F, and/or TeNT. Also, in certain embodiments, the cell has been engineered to express or overexpress a variant or fragment of synaptotagmin that binds BoNT/B and/or BoNT/G.

In certain embodiments, the variant is a protein that has an amino acid sequence that has at least 80%, 85%, 90%, 95%, 98%, or 99% sequence identity with that of protein receptor that binds clostridial neurotoxin. In certain such embodiments, the variant is a protein that has an amino acid sequence that has at least 80%, 85%, 90%, 95%, 98%, or 99% sequence identity with SV2 (e.g., SV2A (SEQ ID NO: 8), SV2B (SEQ ID NO: 9), and SV2C (SEQ ID NO: 10)) or synaptotagmin (e.g., synaptotagmin I (SEQ ID NO: 14) and synaptotagmin II (SEQ ID NO: 15)).

The fragment may, for example, have 50 amino acids or less, 40 amino acids or less, 30 amino acids or less, 20 amino acids or less, or 10 amino acids or less.

In certain embodiments, the variant or fragment comprises the domains of the wild-type protein receptors that bind to the neurotoxin. For example, the variant or fragment may comprise the luminal domain(s) of wild-type SV2 (e.g., SV2A, SV2B, and SV2C) or wild-type synaptotagmin (e.g., synaptotagmin I and synaptotagmin II). In certain such embodiments, the variant or fragment may comprise the fourth luminal domain of wild-type SV2, for example the fourth luminal domain of SV2A (SEQ ID NO: 11), the fourth luminal domain of SV2B (SEQ ID NO: 12), or the fourth luminal domain of SV2C (SEQ ID NO: 13).

Assays are known in the art that can be used to determine which variants or fragments have the desired clostridial neurotoxin binding activity. For example, the skilled artisan would know that an assay may be used to determine whether a variant or fragment of SV2C has the ability to bind BoNT/A.

The skilled artisan would understand that the aforementioned enzymes may also be encoded by nucleic acids that differ from the aforementioned exogenous nucleic acid by conservative substitutions, which are known in the art. The skilled artisan would also understand that variants of the protein receptor may, for example, be encoded by nucleic acids that have at least 80%, 85%, 90%, 95%, 98%, or 99% sequence identity with the nucleic acid encoding the wild-type protein receptor. Thus the invention also contemplates a cell that has been genetically engineered to contain an exogenous nucleic acid that has at least 80%, 85%, 90%, 95%, 98%, or 99% sequence identity with a nucleic acid that encodes one of the aforementioned protein receptors and/or a nucleic acid that differs from the wild-type nucleic acid encoding such a protein receptor by only conservative substitutions, wherein the encoded protein is the wild-type protein receptor or a variant that retains the ability to bind clostridial neurotoxin.

SV2C is the most sensitive to BoNT/A. As such, in certain embodiments where sensitivity to BoNT/A is desired, the cell is genetically engineered to express or overexpress SV2C or a variant or fragment thereof that is capable of binding BoNT/A. SV2C, however, does not bind BoNT/E, which instead binds SV2A and SV2B. As such, in certain embodiments where sensitivity to BoNT/E is desired, the cell is genetically engineered to express or overexpress SV2A and/or SV2B, or variants or fragments thereof that are capable of binding BoNT/E.

The present invention contemplates that the cell may be engineered to express or overexpress two or more proteins receptors, two or more enzymes of the ganglioside synthesis pathway, or protein receptor(s) and enzyme(s) of the ganglioside synthesis pathway. For example, the cell may be engineered to express or overexpress SV2A and SV2C. Such a cell may, for example, have increased sensitivity to BoNT/A and BoNT/E. Also, a cell may be engineered to express or overexpress GD3 synthase and SV2A and/or SV2C.

In addition, it is known that chimeric receptors are capable of binding neurotoxins. For example, chimeric receptors that comprise a domain of an aforementioned protein receptor that binds to the neurotoxin (e.g., the fourth luminal domain of SV2) fused to the transmembrane domain of another receptor, such as a LDL receptor, are known to bind to BoNT and allow for its internalization into the cell. The present invention thus also contemplates engineering cells to express such chimeric receptors.

The cell used in the present invention may be any cell, prokaryotic or eukaryotic, capable of expressing a ganglioside and/or a protein receptor as described above. Examples of such cells include neuronal cells, neuroendocrine cells (e.g., PC12), embryonic kidney cells (e.g. HEK293 cells), breast cancer cells (e.g., MC7), neuroblastoma cells (e.g., Neuro2a (N2a), M17, IMR-32, N18, and LA-N-2 cells), and neuroblastoma-glioma hybrid cells (e.g., NG108 cells). In certain embodiments, the cell is a neuroblastoma or neuroblastoma-glioma cell. In certain embodiments, the cell is an NG108, M17, or IMR-32 cell. In a particular embodiment, the cell is an NG108 cell.

Cells engineered to express or overexpress clostridial toxin receptors may be further selected for increased sensitivity using directed evolution. In this process, cells are exposed to clostridial neurotoxin and the cells that exhibit sensitivity to the lower concentrations of clostridial neurotoxin (as determined, for example, by exhibiting cleavage of an indicator protein therein), as compared to other cells, are selected. These cells are expected to have a greater sensitivity to clostridial neurotoxin than the cells at large. This process can be repeated with lower and lower concentrations of clostridial neurotoxin with the cells exhibiting sensitivity to the lower concentrations being selected.

The selected cells with each round will have increasing sensitivity to clostridial neurotoxin.

As discussed previously, the cell of the present invention may be used in an assay for determining the activity of a polypeptide (e.g., a modified or recombinant clostridial neurotoxin). Such an assay involves contacting the cell with the polypeptide and testing for the presence of product(s) resulting from the cleavage of a SNARE protein in the cell.

The term “contacting” as used herein refers to bringing the cell and the clostridial neurotoxin in physical proximity as to allow physical and/or chemical interaction. Contacting is carried out under conditions and for a time being sufficient to allow interaction of the polypeptide and a protein that is susceptible to proteolysis by wild-type clostridial neurotoxin (e.g., a SNARE protein).

In certain embodiments, such contacting may be by culturing the cell in media containing the polypeptide. The polypeptide is typically present in the media at a concentration of 0.0001 nM to 10,000 nM, 0.0001 to 1,000 nM, 0.0001 to 100 nM, 0.0001 to 10 nM, 0.0001 to 1 nM, 0.0001 to 0.1 nM, 0.0001 to 0.01 nM, or 0.0001 to 0.001 nM. Such culturing may, for example, be for 2 hours or more, 4 hours or more, 6 hours or more, 12 hours or more, 18 hours or more, 24 hours or more, 30 hours or more, 36 hours or more, 40 hours or more, or 48 hours or more.

In certain other embodiments, such contacting may be by transfecting the cell (e.g., transient transfection) with exogenous nucleic acid encoding the polypeptide.

To allow for use in such an assay, the cell comprises a protein that is susceptible to proteolysis by a wild-type clostridial neurotoxin. These proteins will be referred to herein as “indicator protein(s).” The indicator protein may be endogenous (e.g., an endogenous SNARE protein) or the cell may be genetically engineered to express or overexpress an indicator protein.

As discussed above, it is known that SNARE proteins such as SNAP-25, synaptobrevin, and syntaxin are susceptible to proteolysis by clostridial neurotoxins. For example, BoNT/A, BoNT/C, and BoNT/E are known to cleave SNAP-25, BoNT/C is also known to cleave syntaxin, and the other BoNT serotypes and TeNT are known to cleave synaptobrevin. Therefore, the present invention contemplates that such an indicator protein may comprise the amino acid sequence of such a SNARE protein. The invention also contemplates that the indicator protein may instead comprise the amino acid sequence of a variant or fragment of such a SNARE protein, provided that the variant or fragment is susceptible to proteolysis by a wild-type clostridial neurotoxin. In certain embodiments, the variant may have at least 80%, at least 85%, at least 90%, or at least 95% sequence identity with a SNARE protein. The portion of the indicator protein having the amino acid sequence of a SNARE protein, or a variant or fragment thereof, will herein be referred to as the “SNARE domain” of the indicator protein.

The term “susceptible to proteolysis” means that the protein is proteolytically cleavable by the protease component of a wild-type clostridial neurotoxin. In other words, such a protein comprises a protease recognition and cleavage site allowing it to be recognized and cleaved by the protease component of a wild-type clostridial neurotoxin.

In certain embodiments, the indicator protein is labeled. For example, U.S. Pat. No. 8,940,482 to Oyler et al. describes a cell-based assay for assessing the activity of a clostridial neurotoxin wherein the cell has been engineered to express a labeled fusion protein comprising a fluorescent protein domain fused to SNAP-25. The fluorescent protein domain is C-terminal to the SNAP-25 domain and becomes part of the C-terminal fragment that results following cleavage of SNAP-25 by the clostridial neurotoxin. In the assay described by Oyler, the full-length fusion protein is not readily degraded in the cell but the resulting C-terminal fragment is, resulting in the degradation of the fluorescent protein. This is due to the presence in SNAP-25 of a residue that serves as a degron only when it is exposed, by cleavage, at the N-terminal of a resulting fragment. Such “N-degrons” are tagged by ubiquitin ligases and thus the fragment is targeted by proteasomes for degradation.

The present invention therefore contemplates embodiments, such as that described in Oyler, wherein a cell is engineered to express a labeled indicator protein that, in full-length form, is not readily degraded. In such embodiments, cleavage results in a labeled fragment that is readily degraded in the cell (e.g., due to the presence of an N-degron). The indicator protein is labeled on the portion that forms the fragment that is readily degraded and the label is degraded along with the fragment. In such embodiments, the ability of a polypeptide to cleave a SNARE protein in a cell may be determined by the presence (or lack thereof) of the signal from the label following the contacting of the cell with the polypeptide.

In certain such embodiments, the indicator protein also includes a label on the portion of the indicator protein that, following cleavage, forms a fragment that does not degrade as readily as the other fragment. For example, the indicator protein may be a fusion protein comprising two labels and a SNARE domain with the labels flanking the SNARE domain. In embodiments wherein the full-length indicator protein is not readily degraded in the cell but, following cleavage thereof, one of the resulting fragments is, cleavage of the SNARE protein may be determined by comparing the signal obtained from the label on the readily degradable fragment with the signal from the label on the less readily degradable fragment. For example, in embodiments such as that in Oyler where the C-terminal fragment resulting from cleavage is readily degradable but the N-terminal fragment is not, cleavage can be determined by comparing the signal obtained from the label on the C-terminal fragment with the signal from the label on the N-terminal fragment. In such embodiments, labels emitting fluorescent signals that are more clearly distinguishable from each other (e.g., red and green or red and cyan) may be chosen.

The term “label”, as used herein, means a detectable marker and includes e.g. a radioactive label, an antibody and/or a fluorescent label. The amount of test substrate and/or cleavage product may be determined, for example, by methods of autoradiography or spectrometry, including methods based on energy resonance transfer between at least two labels such as a FRET assay (discussed further below). Alternatively, immunological methods such as western blot or ELISA may be used for detection.

Examples of labels that may be used in the practice of the present invention include: radioisotopes; fluorescent labels; phosphorescent labels; luminescent labels; and compounds capable of binding a labeled binding partner. Examples of fluorescent labels include: yellow fluorescent protein (YFP); blue fluorescent protein (BFP); green fluorescent protein (GFP), such as NeonGreen; red fluorescent protein (RFP), such as mScarlet; cyan fluorescent protein (CFP); and fluorescing mutants thereof. Examples of luminescent labels include: photoproteins; luciferases, such as firefly luciferase, Renilla and Gaussia luciferases; chemiluminescent compounds; and electrochemiluminescent (ECL) compounds. In embodiments as discussed above wherein an N-terminal label and a C-terminal label are chosen such that the signals emitted are more readily distinguishable from each other, examples of such label pairs may include RFP and GFP and RFP and CFP. For example, a RFP such as mScarlet may serve as the N-terminal label and a GFP such as NeonGreen or a CFP may serve as the C-terminal label.

In certain embodiments, the label is a protein label, such as an antibody, a fluorescent protein, a photoprotein, and a luciferase.

As used herein, “N-terminal label” refers to a label, whether protein or not, located on the portion of the indicator protein that is N-terminal to the clostridial neurotoxin cleavage site and “C-terminal label” refers to a label, whether protein or not, located on the portion of the indicator protein that is C-terminal to the clostridial neurotoxin cleavage site. The label need not be at the N-terminus or the C-terminus of the indicator protein to be termed the N-terminal or C-terminal label. Rather, these terms refer to the positions of the label relative to the clostridial neurotoxin cleavage site. In certain embodiments of the present invention, RFP, such as mScarlet, is used as the N-terminal label and GFP, such as NeonGreen, or CFP is used as the C-terminal label.

Another assay is a Fluorescence Resonance Energy Transfer (FRET) assay. In such an assay, the indicator protein comprises a donor label on one side of a cleavage site and an acceptor label on the other side. The donor label absorbs energy and subsequently transfers it to the acceptor label. The transfer of energy results in a reduction in the fluorescence intensity of the donor chromophore and an increase in the emission intensity of the acceptor chromophore. Cleavage of the substrate results in less successful transfer of energy. Thus, a successful cleavage can be determined based on the reduced ability for this transfer to take place. In such embodiments, YFP and CFP may be paired as a FRET pair, as can RFP and GFP.

In certain embodiments of the present invention, the indicator protein is a fusion protein that comprises a SNARE domain. The fusion protein may also comprise additional domains such as a label domain. The label domain may have the amino acid sequence of a protein label. An example of such a fusion protein comprises: an N-terminal label domain, such as the amino acid sequence for mScarlet; a SNARE domain, such as the amino acid sequence for SNAP-25; and a C-terminal label domain, such as the amino acid sequence for NeonGreen.

The fusion protein may also comprise other domains such as a selection marker (discussed further below). In such embodiments, the selection marker domain may be separated from the portion of the fusion protein containing the remaining domains (e.g., the SNARE domain and the label domain(s)) by a linker that may be cleaved to allow for separation of the selection marker and the remainder of the indicator protein following translation. The linker may, for example, be self-cleaving (e.g., a 2A self-cleaving peptide).

As discussed previously, the cell may be engineered to express or overexpress an indicator protein. The skilled artisan would know which nucleic acids may be used to allow for such expression and methods to engineer such cells to express such indicator proteins. An example of such a nucleic acid is SEQ ID NO: 1, which expresses a fusion protein having mScarlet as an N-terminal label, SNAP-25 as a SNARE domain, NeonGreen as a C-terminal label, luciferase as an additional label domain, puromycin-N-acetyltransferase as a selection marker, and a 2A self-cleaving peptide. Another example of such a nucleic acid is SEQ ID NO: 2, which expresses a fusion protein protein having mScarlet as an N-terminal label, SNAP-25 as a SNARE domain, CFP as a C-terminal label, luciferase as an additional label domain, puromycin-N-acetyltransferase as a selection marker, and a 2A self-cleaving peptide.

The present invention also relates in part to a method for making the aforementioned genetically-engineered cell. The method involves introducing an exogenous nucleic acid encoding a protein of interest into a cell. The protein of interest may be, for example: a clostridial neurotoxin receptor or a variant or fragment thereof having the ability to bind clostridial neurotoxin; an enzyme of the ganglioside synthesis pathway or a variant or fragment thereof having the catalytic activity of such enzyme; and/or an indicator protein.

In certain embodiments, the method involves transforming a cell with a nucleic acid encoding a protein of interest. Such transformation may be by transfection.

In certain embodiments, the nucleic acid encodes a fusion protein comprising two or more domains, with each domain having the amino acid sequence for a protein of interest or other components of the fusion protein. For example, a nucleic acid may encode a fusion protein comprising the amino acid sequence for a protein receptor (e.g., SV2A or SV2C) and the amino acid sequence for an enzyme of the ganglioside synthesis pathway (e.g., GD3 synthase). In another example, a nucleic acid may encode a fusion protein comprising the amino acid sequence for a protein receptor, the amino acid sequence for an enzyme of the ganglioside synthesis pathway, and the amino acid sequence for a selection marker. In yet another example, a nucleic acid may encode a fusion protein comprising the amino acid sequence for a protein receptor, the amino acid sequence for an enzyme of the ganglioside synthesis pathway, the amino acid sequence for an indicator protein, and the amino acid sequence for a selection marker.

In such embodiments, the domains may be separated from each other by linkers. The linkers may, for example, be cleavable by enzymes in the cell or contain a self-cleaving peptide (e.g., 2A self-cleaving peptide), allowing for the individual domains to form separate proteins in the cell.

The nucleic acid may optionally comprise regulatory elements. The term “regulatory elements” as used herein refers to regulatory elements of gene expression, including transcription and translation, and includes elements such as TATA boxes, promoters, enhancers, ribosome binding sites, Shine-Dalgarno sequences, IRES regions, polyadenylation signals, terminal capping structures, and the like. The regulatory element may comprise one or more heterologous regulatory elements or one or more homologous regulatory elements. A “homologous regulatory element” is a regulatory element of a wild-type cell, from which the nucleic acid molecule is derived, which is involved in the regulation of gene expression of the nucleic acid molecule or the polypeptide in the wild-type cell. A “heterologous regulatory element” is a regulatory element which is not involved in the regulation of gene expression of the nucleic acid molecule or the polypeptide in the wild-type cell. Regulatory elements for inducible expression, such as inducible promoters, may also be used.

The nucleic acid molecule can be, for example, hnRNA, mRNA, RNA, DNA, PNA, LNA, and/or modified nucleic acid molecules. The nucleic acid molecule can be circular, linear, integrated into a genome or episomal. Also, concatemers coding for fusion proteins comprising three, four, five, six, seven, eight, nine or ten polypeptides are encompassed. Moreover, the nucleic acid molecule may contain sequences encoding signal sequences for intracellular transport such as signals for transport into an intracellular compartment or for transport across the cellular membrane.

The nucleic acid may be designed to provide high levels of expression in the host cell. Methods of designing nucleic acid molecules to increase protein expression in host cells are known in the art, and include decreasing the frequency (number of occurrences) of “slow codons” in the encoding nucleic acid sequence.

The nucleic acid may be introduced using any means known in the art. For example, it may be included in a vector (e.g., a plasmid) used to introduce the nucleic acid into a cell.

Any vector known in the art to allow for expression of the nucleic acid in a cell may be used. The vector may be suitable for in vitro and/or in vivo expression of the protein of interest. The vector can be a vector for transient and/or stable gene expression. The vector may additionally comprise regulatory elements and/or selection markers. The vector may, for example, be artificial or be of viral origin, of phage origin, or of bacterial origin. Examples of vectors for use in the present invention include adenoviral vectors, vaccinia vectors, SV-40 viral vectors, retroviral vectors, λ-derivates, and plasmids. Examples of plasmids for use in the present invention include plasmids having a pD2500 or pcDNA3.1 backbone.

Methods for using vectors to introduce nucleic acid into a cell are known in the art. See Laura Bonetta, “The Inside Scoop—Evaluating Gene Delivery Methods,” Nature Methods 2: 875-883 (2005).

The host cell may comprise an inducer of expression of the protein of interest. Such an inducer of expression may be a nucleic acid molecule or a polypeptide or a chemical entity, including a small chemical entity. The inducer of expression may, for example, increase transcription or translation of a nucleic acid molecule encoding the protein of interest. The inducer may, for example, be expressed by recombinant means known to the skilled artisan. Alternatively, the inducer may be isolated from a cell, for example a clostridial cell.

In certain embodiments, cells that have been successfully transformed may be determined by determining the presence of a selection marker. In such embodiments, the vector containing the exogenous nucleic acid encoding the desired protein may also contain nucleic acid encoding a selection marker.

In certain embodiments, the selection marker is a detectable tag. Examples of such tags include a His tag, a GST tag, a Strep tag, and an SBP tag. The tag may be expressed as part of a fusion protein that also comprises the protein of interest. In such embodiments, the tag may be flanked by one or more protease cleavage sites or self-cleaving peptides. Such allow for the tag to be cleaved from the protein following translation.

In certain other embodiments, the selection marker confers resistance to an antibiotic. Examples of such selection markers include: puromycin-N-acetyltransferase (resistance to puromycin), aminoglycoside 3′f3-phosphotransferase (resistance to G418), blasticidin S deaminase (resistance to Blasticidin S), and hygromycin B phosphotransferase (resistance to hygromycin B). Successful transformation of the cell may thus be determined by exposing the cell to the relevant antibiotic.

In certain embodiments, the successful transformation of cells that are genetically engineered to express or overexpress a ganglioside and/or a protein receptor that bind a clostridial neurotoxin may be determined by contacting such cells with the clostridial neurotoxin and determining whether cleavage of an indicator protein therein has taken place.

The present invention further relates to the use of the aforementioned genetically-engineered cell in an assay to determine the biological activity of a polypeptide, for example, a modified or recombinant clostridial neurotoxin.

Biological activity of such polypeptides can be measured by various tests, all of which are known to the person skilled in the art.

As discussed previously, the assay involves contacting the cell with the polypeptide under conditions and for a period of time that would allow the protease domain of a wild-type clostridial neurotoxin to cleave an indicator protein in the cell and determining the presence of products resulting from the cleavage of the indicator protein. The indicator protein may be endogenous to the cell (e.g., an endogenous SNARE protein) or may be an exogenous indicator protein of the type described previously.

Such assays typically also involve a step of determining the degree of conversion of the indicator protein into its cleavage product(s). The observation of one or more cleavage product(s) generated after contacting the polypeptide with the indicator protein or the observation of an increase in the amount of cleavage product(s) is indicative of proteolytic activity of the polypeptide.

The step of determining may involve comparing full-length indicator protein and cleavage product(s). The comparing may involve determining the amount of full-length indicator protein and/or the amount of one or more cleavage product(s) and may also involve calculating the ratio of full-length indicator protein and cleavage product(s). In addition, the assay for determining the proteolytic activity may comprise a step of comparing the cleavage product(s) that appear following the contact of the polypeptide being assayed and the indicator protein and a control. The control may, for example, be the cleavage product(s) that appear following the contact of a clostridial neurotoxin that is known to be capable of cleaving the same indicator protein.

In certain embodiments, following contacting of a cell with the polypeptide, the cell may be lysed and analyzed by gel electrophoresis and Western blotting. For example, anti-SNAP-25 antibody that binds to the N-terminus of SNAP-25 may be used in a Western blot to determine the presence of full-length SNAP-25 and cleaved SNAP-25 (which would migrate in a separate band from full-length SNAP-25).

Methods and techniques used to lyse host cells, such as bacterial cells are known in the art. Examples include ultrasonication or the use of a French press.

In certain embodiments, the full-length indicator protein is not readily degraded in the cell but, following cleavage thereof, one of the resulting fragments is. This may, for example, be due to the presence of a residue that serves as a degron only when it is exposed, by cleavage, at the N-terminal of a resulting fragment.

In such embodiments, the indicator protein may be labeled on the portion thereof that is more readily degraded following cleavage. The label should be chosen so that, when degradation of the fragment occurs, the label is also degraded. In such embodiments, whether cleavage occurs can be determined based on measuring the signal from the label.

The signal received may be compared to a control.

In certain such embodiments wherein another fragment formed following cleavage is not as readily degraded, the indicator protein may also include a label on the portion of the indicator protein that, following cleavage, forms that fragment. In such embodiments, whether cleavage occurs can thus be determined by comparing the signal from the label on the more readily-degradable fragment with the signal from the label on the less readily-degradable fragment, which serves as a control.

The signal(s) from the label(s) may be analyzed using fluorescent-activated cell sorting (FACS). For example, in an embodiment wherein the full-length indicator protein and the N-terminal fragment thereof formed following cleavage are not readily degradable within the cell but the C-terminal fragment resulting from cleavage is and the N-terminal label is mScarlet and the C-terminal label is NeonGreen, FACS analysis of the cells after successful cleavage will show lower green emission as compared with red emission. By contrast, if cleavage does not occur, red and green fluorescence should be equally prevalent.

In the alternative, fluorescent photomicrographs may be taken of the cells. In an embodiment such as that described above, successful cleavage would result in less green fluorescence emitted in cells as compared to control cells that have not been exposed to protease. By contrast, red fluorescence should remain the same as control.

Also, in certain embodiments, the assay may be a FRET assay. As discussed previously, in such an assay the indicator protein comprises an N-terminal label and a C-terminal label with one label being a donor label and the other being an acceptor label. Transfer of energy between the donor label and the acceptor label results in a reduction in the fluorescence intensity of the donor label and an increase in the emission intensity of the acceptor label. The success of such a transfer is dependent on the labels remaining in close proximity. Cleavage of the indicator protein tends to render these labels more distant and thus such a transfer less successful. Successful cleavage can therefore be determined based on the reduced ability for the transfer of energy to take place.

In addition to the above, any other means known in the art by which to analyze fluorescence from indicator proteins to determine whether cleavage has occurred may be used in the practice of the present invention.

In certain embodiments, a polypeptide is deemed proteolytically active if 20% or more, 50% or more, 75% or more, 80% or more, 90% or more, 95% or more, 97% or more, 98% or more, or 99% or more of the indicator protein is converted into the cleavage product(s) in less than 1 minute, less than 5 minutes, less than 20 minutes, less than 40 minutes, less than 60 minutes, or less than 120 minutes.

Cleavage may be measured at intervals in order to follow the catalytic activity over time.

All references cited in this specification are herewith incorporated by reference with respect to their entire disclosure content and the disclosure content specifically mentioned in this specification.

### EXAMPLES

### Example 1—Selection of Optimum Parental Cell Line for Creation of Indicator Cell Line

Neuro2A (N2a; ATCC CCL-131), BE(2)-M17 (M17; ATCC CRL-2267), IMR-32 (ATCC CCL-127), and NG108-15 [108CC15] (ATCC HB-12317) cells were studied for the purpose of choosing the optimum parental cell line for the development of the stable transfected cell line.

Upon delivery, the cells were allowed to recover and grow. Stocks of the cells were then frozen down and stored in liquid nitrogen. Once sufficient vials of cell stocks were made, the cells were assayed for their sensitivity to BoNT/A.

The cells were cultured in media containing BoNT/A (Metabiologics, Inc.) for 8 or 24 hours. The cells cultured for 8 hours were cultured in media containing 0.1 nM, 1 nM, or 10 nM BoNT/A. The cells cultured for 24 hours were cultured in media containing 1 nM, 0.1 nM, or 0.01 nM BoNT/A.

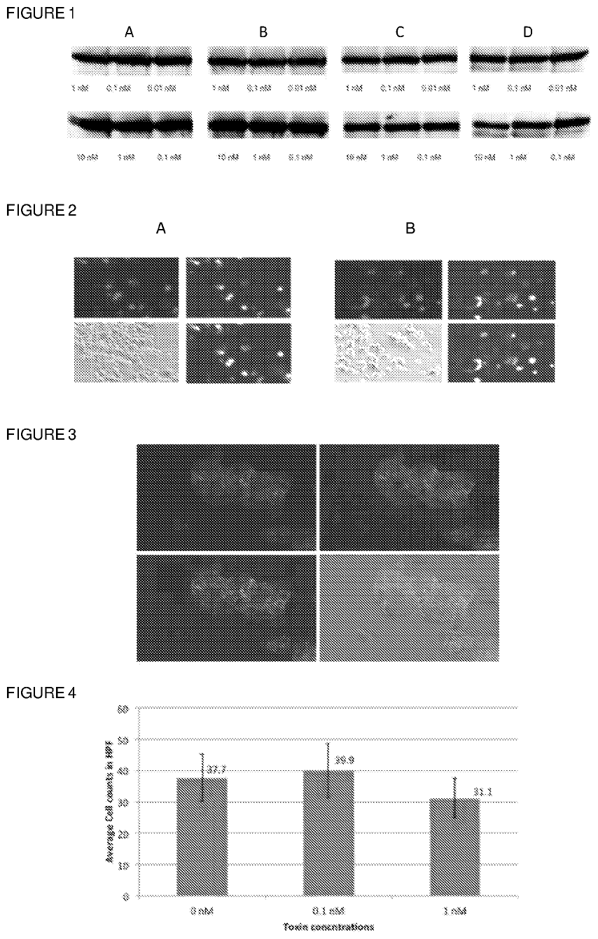

Cleavage of endogenous SNAP-25 was analyzed by Western blot using an anti-SNAP-25 antibody (Sigma #S9684) with standard protocols (FIG. 1). The NG108 cells showed greater sensitivity to BoNT/A than the other cells, with the N2a cells being the least sensitive. The NG108 cell line was therefore chosen as the primary candidate for the development of a stably transfected indicator cell line. The M17 and IMR-32 cell lines showed similar sensitivity to BoNT/A at the concentrations and times tested. Due to ease of culture and familiarity, M17 was chosen as a backup for the NG108.

### Example 2—Transfection of Cells with Plasmid Containing Indicator Construct

The sensitivities of NG108 cells and M17 cells to puromycin (InvivoGen #ANT-PR) and G418 (VWR #97064-358) were determined. The cells were grown to ˜50% confluency and then cultured with various concentrations of puromycin and G418. Both cell lines showed similar sensitivity to puromycin and G418.

Plasmids (pD2500; Atum) were engineered to contain nucleic acid sequences encoding puromycin-N-acetyltransferase (PuroR), a chimeric protein, and a 2A self-cleaving peptide. In the expressed product, the 2A self-cleaving peptide was located between PuroR and the chimeric protein. The chimeric protein contained SNAP-25 flanked between N-terminal and C-terminal fluorescent proteins and luciferase (located at the C-terminus). PuroR conferred resistance to puromycin. Luciferase allowed for luminescent measurements of degradation in addition to the fluorescent-based measurements of degradation facilitated by the fluorescence proteins. The N-terminal fluorescent protein was mScarlet and the C-terminal fluorescent protein was either NeonGreen, a green fluorescent protein, or Cyan Fluorescent Protein (CFP). The plasmid insert containing the nucleic acids encoding PuroR, the 2A self-cleaving peptide, and the construct containing mScarlet, SNAP-25, NeonGreen, and luciferase (mScarlet-SNAP25-GeNluc) had the nucleotide sequence of SEQ ID NO: 1. The plasmid insert containing nucleic acids encoding PuroR, the 2A self-cleaving peptide, and the construct containing mScarlet, SNAP-25, CFP, and luciferase (mScarlet-SNAP25-CyanNluc) had the nucleotide sequence of SEQ ID NO: 2.

NeonGreen was chosen due to its excitation/emission spectrum and intensity. In case NeonGreen did not degrade well upon cleavage of the indicator protein, CFP was chosen as a backup due to previous data indicating that it is rapidly degraded when the indicator protein is cleaved.

NG108 cells and M17 cells were transfected with plasmids containing either the mScarlet-SNAP25-GeNluc construct or the mScarlet-SNAP25-CyanNluc construct. Transfection was with Lipofectamine 3000 (ThermoFisher) or polyethyleneimine using standard protocols.

Twenty-four hours post-transfection, cells were analyzed by fluorescent microscopy to ascertain efficiency of transfection and correct expression of the indicator protein (FIG. 2A-B, examples of cells expressing indicator protein containing NeonGreen shown). Due to over-expression, much of the indicator protein was cytosolic. The red and green representing, respectively, the N- and C-terminal ends of the indicator protein were easily detected and indicated that the terminal ends co-localized. High transient expression was seen in both cell types with >70% transfection efficiency.

Upon confirmation that the cells were efficiently transfected and the indicator proteins were expressed correctly, the transfected cells were selected with either 2.5 μg/ml puromycin or “shocked” with an initial high dose of 20 μg/ml puromycin for 1 day and then cultured in 5-10 μg/ml puromycin. Both treatments yielded a pool of fluorescent, puromycin-resistant cells.

Concurrent with this, a number of additional transfections with both indicator constructs were done and selection for puromycin resistance conducted, yielding further pools of fluorescent, puromycin-resistant cells. This ultimately resulted in about 6 independent pools of fluorescent, puromycin-resistant NG108 cells and 2 independent pools of puromycin-resistant, fluorescent M17 cells. The pools were expanded, and stocks frozen down and tested for thaw viability.

The puromycin-resistant cells were analyzed to confirm stable transfection of the indicator construct (FIG. 3). In NG108 cells stably transfected with the indicator construct containing NeonGreen, both mScarlet (red) and NeonGreen (green) co-localized. This indicated that the full-length intact protein was made and distributed within the cell. Furthermore, the fluorescence was predominantly seen on the cell membrane, indicating the protein was correctly localized (due to the presence of SNAP-25).

### Example 3—Confirmation of Cleavage of Indicator Protein

Plasmids (pcDNA3.1) were engineered to contain SEQ ID NO: 3 which encodes the BoNT/A light chain, CFP, and an N-terminal SBP tag. The nucleic acid encoding the BoNT/A light chain was synthesized using DNA2.0 (Atum).

Cells from Example 2 stably transfected with the indicator constructs (mScarlet-SNAP25-GeNluc or mScarlet-SNAP25-CyanNluc) were transiently transfected with an expression vector containing the CFP-BoNT/A construct. Numerous red but not green or cyan cells were demonstrated at 24 and 48 after transfection, indicating that the indicator protein was cleaved and the C-terminal fragment rapidly degraded.

### Example 4—Confirmation of Cleavage of Indicator Protein

NG108 cells from Example 2 stably transfected with the mScarlet-SNAP25-GeNluc indicator construct were plated in 96-well optical plates (ThermoFisher #165305) (20-30 K cells per well) in complete DMEM media (Corning #50-013-PB) and allowed to adhere for 4 hours. The media was then changed to Neurobasal Plus (ThermoFisher #A35829) and the cells cultured for a further 20 hours after which the media was changed for Neurobasal Plus media containing BoNT/A at 0 (control), 0.1, or 1.0 nM. The cells were cultured for a further 24 hours. The cells were then trypsinized, washed once in media, and resuspended in DMEM/FBS media or DPBS with 10 units Benzonase/ml at ˜2×10⁶ cells/ml. The cells were then analysed on a SY3200 (Sony Biotechnologies) cell sorter using appropriate lasers/filters for NeonGreen and mScarlet.

FIG. 4 depicts the number of green cells per HPF for mScarlet-SNAP25-GeNluc expressing NG108 cells after treatment with BoNT/A for 24 hours. An approximately 25% reduction in green-positive cells per HPF was indicated in the pool of cells treated with 1 nM BoNT/A.

### Example 5—Confirmation of Cleavage of Indicator Protein

Representative NG108 and M17 cells from Example 2 stably transfected with the mScarlet-SNAP25-GeNluc indicator construct were trypsinized, washed once in media, and resuspended in DMEM/FBS media or DPBS with 10 units Benzonase/ml at ˜2×10⁶ cells/ml. The cells were then analyzed on a SY3200 (Sony Biotechnologies) cell sorter using appropriate lasers/filters for NeonGreen and mScarlet.

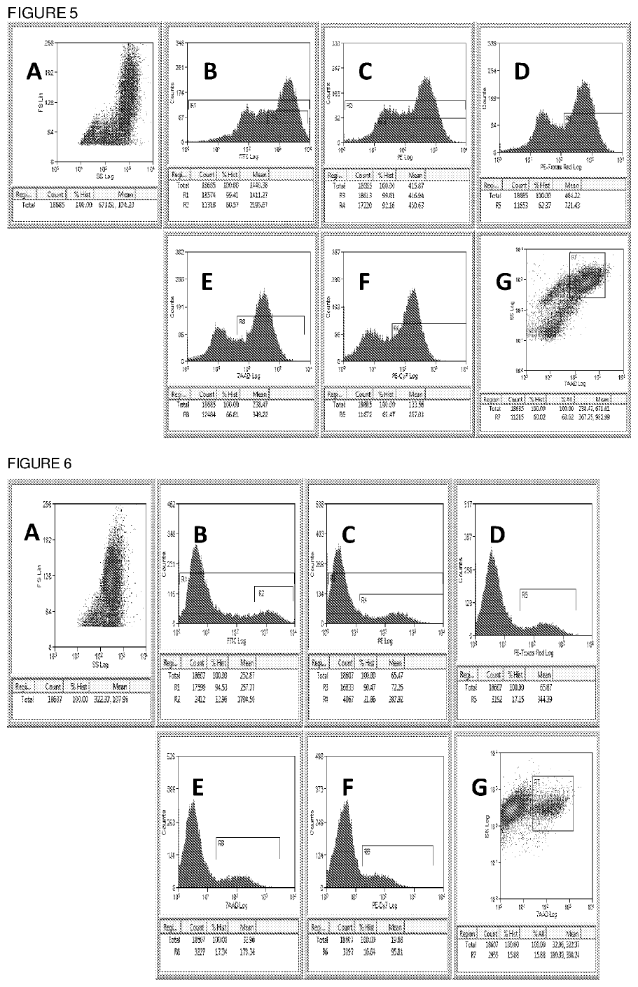

NG108 cells were excited at 488 nm to directly excite NeonGreen while minimally exciting mScarlet. Fluorescence emission was measured at different wavelengths. In addition, side- and forward-scattered light intensity was measured to identify sub-populations of cells. FIG. 5A depicts a scatter plot showing side-scatter (SS) on the x axis and forward-scatter (FS) on the y axis: the distribution illustrates variation in cell granularity/complexity (SS) and cell size (FS). FIG. 5B depicts a histogram showing cell distribution of emission fluorescence intensity measured at 525 nm (FITC filter). FIG. 5C depicts a histogram showing cell distribution of emission fluorescence intensity measured at 585 nm (PE filter). FIG. 5D depicts a histogram showing cell distribution of emission fluorescence intensity measured at 617 nm (PE-Texas Red filter). FIG. 5E depicts a histogram showing cell distribution of emission fluorescence intensity measured at 665 nm (7AAD filter). FIG. 5F depicts a histogram showing cell distribution of emission fluorescence intensity measured at 785 nm (PE-Cy7 filter). FIG. 5G depicts a scatter plot showing cell emission fluorescence of cells measured at 665 nm (7AAD filter) on the x axis and side-scatter (SS) on the y axis. The histograms show two distinct peaks of fluorescence with the less fluorescent peak representing non-expressing cells and the more fluorescent peak representing cells expressing the indicator protein. At all wavelengths of emission reading, the fluorescence remains high. This included when emission was measured at 785 nm (FIG. 5F), at which most of the emitted light is due to FRET, thus confirming FRET between NeonGreen and mScarlet. The percentage fraction of high fluorescent cells in the N108 pool ranged from 61% to 93%.

M17 cells were excited at 488 nm to directly excite NeonGreen while minimally exciting mScarlet. Fluorescence emission was measured at different wavelengths. In addition, side- and forward-scattered light intensity was measured to identify sub-population of cells. FIG. 6A depicts a scatter plot showing side-scatter (SS) on the x axis and forward-scatter (FS) on the y axis: the distribution illustrates variation in cell granularity/complexity (SS) and cell size (FS). FIG. 6B depicts a histogram showing cell distribution of emission fluorescence intensity measured at 525 nm (FITC filter). FIG. 6C depicts a histogram showing cell distribution of emission fluorescence intensity measured at 585 nm (PE filter). FIG. 6D depicts a histogram showing cell distribution of emission fluorescence intensity measured at 617 nm (PE-Texas Red filter). FIG. 6E depicts a histogram showing cell distribution of emission fluorescence intensity measured at 665 nm (7AAD filter). FIG. 6F depicts a histogram showing cell distribution of emission fluorescence intensity measured at 785 nm (PE-Cy7 filter). FIG. 6G depicts a scatter plot showing cell emission fluorescence of cells measured at 665 nm (7AAD filter) on the x axis and side-scatter (SS) on the y axis. The histograms show two distinct peaks of fluorescence with the less fluorescent peak representing non-expressing cells and the more fluorescent peak representing cells expressing the indicator protein. At all wavelengths of emission reading, the fluorescence remains high. This included when emission was measured at 785 nm (FIG. 6F), at which most of the emitted light is due to FRET, thus confirming FRET between NeonGreen and mScarlet. The percentage fraction of high fluorescent cells in the M17 pool ranged from 14% to 24%, i.e., the fraction of cells expressing fluorescent proteins in the M17 cell pool was smaller compared to the NG108 cell pool.

NG108 cells from Example 2 stably transfected with the mScarlet-SNAP25-GeNluc indicator construct were treated with BoNT/A at 0 (control), 0.1, or 1.0 nM in the manner described in Example 4.

Fluorescence from stably transfected pools of NG108 cells was measured using a SY3200 (Sony Biotechnologies) Analyzer. Cells were excited at 488 nm to directly excite NeonGreen while minimally exciting mScarlet. Fluorescence emission was measured at 530 nm (FITC filter), which detects NeonGreen fluorescence but not mScarlet fluorescence. FIG. 7A depicts a histogram showing the distribution of emission fluorescence intensity from untreated (control) cells measured at 525 nm. FIG. 7B depicts a histogram showing the distribution of emission fluorescence intensity from cells treated with 0.1 nM BoNT/A measured at 525 nm. FIG. 7C depicts a histogram showing the distribution of emission fluorescence intensity from cells treated with 1 nM BoNT/A measured at 525 nm. Fluorescence of NeonGreen decreased by about 15% in the cell pool (mean fluorescence of cells in Gate R2) after treatment with 1 nM BoNT/A (FIG. 7C) compared to untreated control (FIG. 7A) suggesting that the resulting NeonGreen—containing C-terminal fragment is degraded after cleavage.

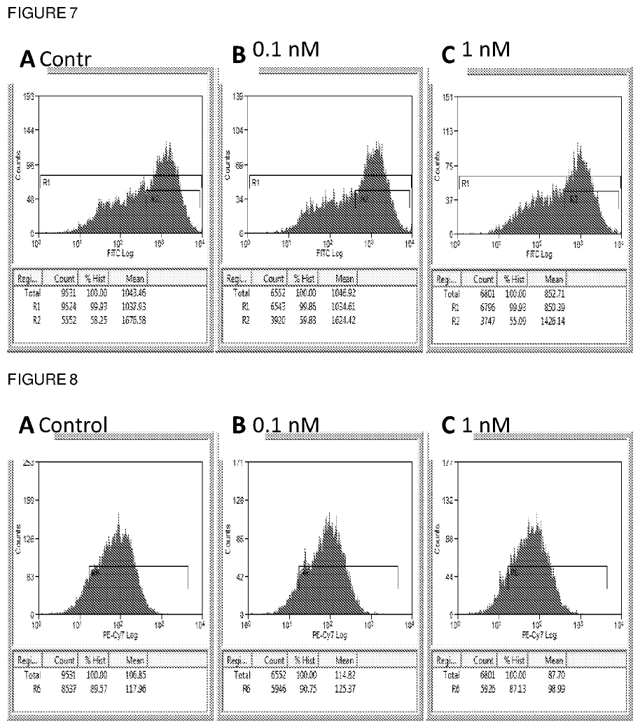

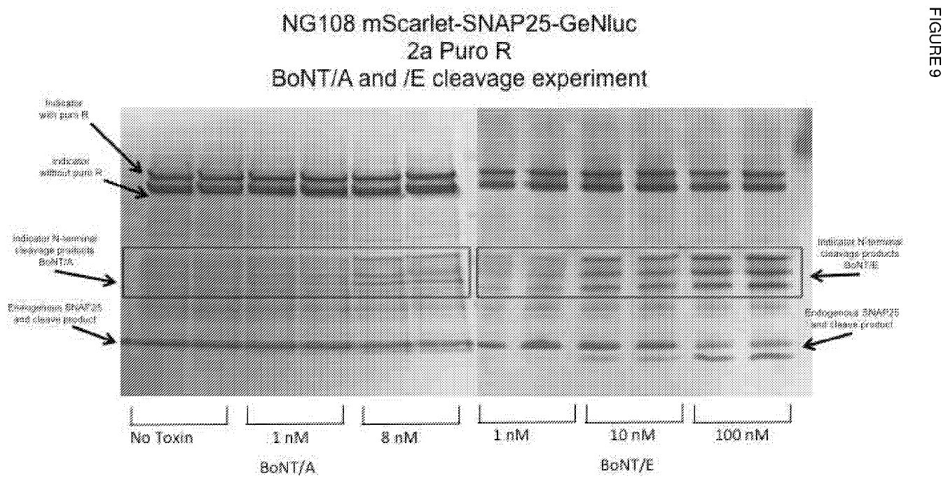

A flow cytometry determination of loss of FRET emission was also conducted. Fluorescence from stably transfected pools of NG108 cells was measured using a SY3200 (Sony Biotechnologies) Analyzer. Cells were excited at 488 nm to directly excite NeonGreen while minimally exciting mScarlet. Fluorescence emission was measured at 785 nm (Cy7 filter), which detects mScarlet fluorescence but not NeonGreen fluorescence. FIG. 8A depicts a histogram showing the distribution of emission fluorescence intensity from untreated (control) cells measured at 785 nm. FIG. 8B depicts a histogram showing the distribution of emission fluorescence intensity from cells treated with 0.1 nM BoNT/A measured at 785 nm. FIG. 8C depicts a histogram showing the distribution of emission fluorescence intensity from cells treated with 1 nM BoNT/A measured at 785 nm. FRET intensity decreased by 16% (mean fluorescence of cells in Gate R6) after treatment with 1 nM BoNT/A (FIG. 8C) compared to the untreated control (FIG. 8A), consistent with NeonGreen degradation.

### Example 6—Confirmation of Cleavage

A Western blot was performed on NG108 cells from Example 2 stably transfected with the mScarlet-SNAP25-GeNluc indicator construct and treated with no toxin (control), 1 nM or 8 nM BoNT/A or 1 nM, 10 nM, or 100 nM BoNT/E in the manner described in Example 4 (FIG. 9).

Cell lines were tested for cleavage of SNAP-25 (either endogenous or exogenous) using standard blotting techniques and a rabbit anti-SNAP25 primary antibody.

Cells were lysed using M-PER reagent (ThermoFisher #78501) according to manufacturer recommendations. The lysate was clarified at 15 kg for 10 minutes and a 10 μl sample run on a NuPage 12% Bis-Tris Gel (ThermoFisher #NP0341BOX) in MOPS buffer (ThermoFisher #NP0001) at 200V. The proteins were transferred to a PVDF membrane (ThermoFisher #LC2005) using the XCell II blotting system and Nu-Page transfer protocol. The resultant blot was blocked in 1% BSA/0.05% Tween20/PBS, primary antibody 1:3,000 anti-SNAP-25, secondary antibody 1:5,000 alkaline phosphatase conjugated goat anti-rabbit (ThermoFisher #31340), and developed in NBT/BCIP substrate (ThermoFisher #34042). The developed blot was scanned and densitometry calculated using ImageJ and plotted in MS Excel.

The indicator protein was detected in all lysates. No apparent cleavage products were detected in the control samples. The cells treated with either BoNT/A or BoNT/E produced cleavage products, increasing with higher dosage. Interestingly, the cells appear to be about as sensitive to BoNT/E as to BoNT/A.

### Example 7—Transfection with Receptor Construct

Plasmids (pD2500; Atum) were engineered to contain nucleic acid encoding the receptor construct GD3-SV2C-Syt and aminoglycoside 3′-phosphotransferase (Neo) (SEQ ID NO: 4). The nucleic acid expressed a fusion protein comprising GD3 synthase, SV2C, and syntaxin, and Neo, with each domain separated from each other by 2A self-cleaving peptides. Syntaxin was engineered into the fusion protein for use with other isoforms of BoNT. Neo conferred resistance to G418.

NG108 cells from Example 2 stably transfected with the mScarlet-SNAP25-GeNluc indicator construct were grown in T75 flasks to ˜60% confluency. They were then transfected with the receptor construct-containing plasmid (2 μg/ml) in 5 ml OptiMem/polyethylenimine overnight according to standard protocol. In the morning, the cells were washed 1× with fresh complete DMEM media and cultured for a further 24 to 48 hours in complete DMEM media. The media was then changed to complete DMEM media with 500 μg/ml G418 and the cells cultured for a further 1 to 2 weeks, with media/G418 changes as needed. Cells were observed to start dying by 3 days post G418 addition and continued for ˜1 week (˜60% cell death). The cells left after ˜2 weeks were G418 resistant.

### Example 8—Directed Evolution of Cells

The cells from Example 7 were subjected to two sorts to isolate those cells that had the highest fluorescence and thus highest expression of the indicator protein.

### Example 9—Directed Evolution of Cells

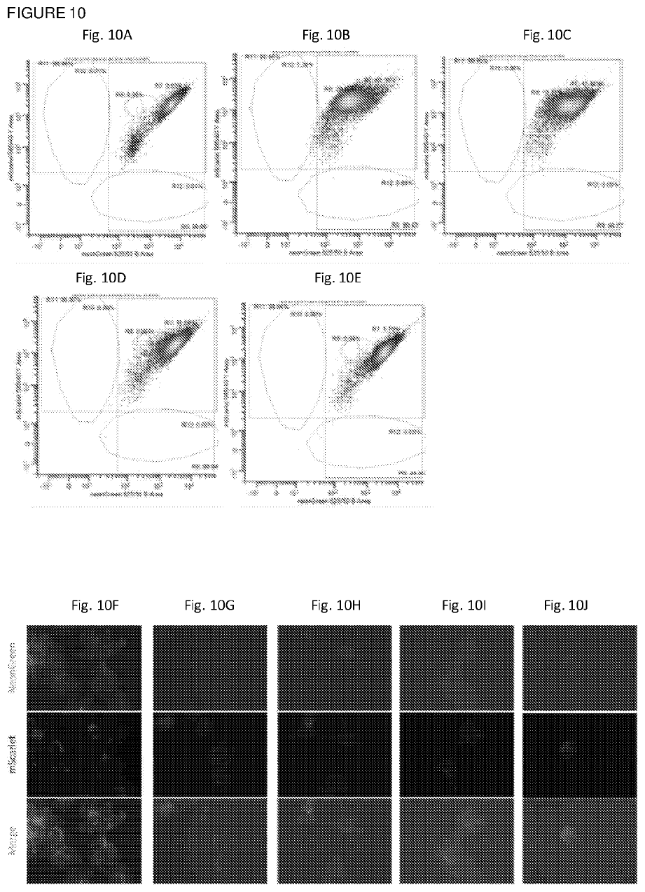

The cells selected for high expression of indicator protein in Example 8 were treated in the fashion described in Example 4 at 0.1 nM, 1 nM, or 10 nM for BoNT/A or 10 nM BoNT/E for 72 hours. After treatment, the cells were washed three times, trypsinized, resuspended in fresh media, and sorted. Screening of the cells showed a clear dose sensitive response to BoNT/A (FIG. 10A). Fluorescent microscopy showed that the cells also decreased in green fluorescence with dose of treatment while maintaining the same level of red fluorescence (FIG. 10B). While the results clearly showed an increase in BoNT/A sensitivity, it was noted that a similar increase was not seen for BoNT/E.

The cells that were sensitive to BoNT/A at 1,000 pM (1 nM) were selected. Example 10—Directed evolution of cells

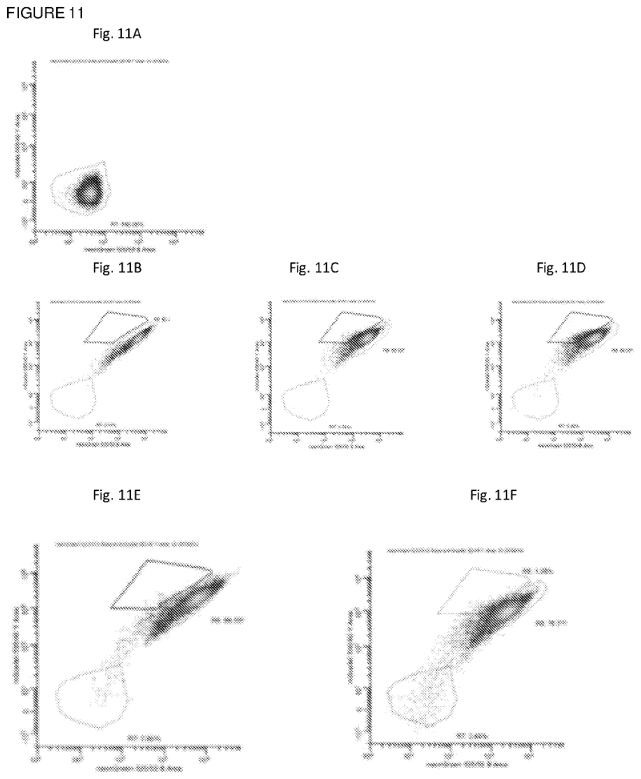

Wild-type NG108 cells (i.e., not transfected with the reporter or receptor constructs) were sorted (FIG. 11A). These cells exhibit neither green nor red fluorescence.

The cells from Example 9 that were sensitive to BoNT/A at 1,000 pM (1 nM) were expanded and treated with 100 pM BoNT/A in the fashion previously discussed or not treated (control). After treatment for 48 or 96 hours, the cells were washed three times, trypsinized, resuspended in fresh media, and sorted. FIGS. 11B-D depict, respectively, the flow cytometry data for the control, the cells treated with 100 pM BoNT/A for 48 hours, and the cells treated with 100 pM BoNT/A for 96 hours.

Cells from Example 8 that were sorted twice for high expression of the indicator construct but not subject to the sorting in Example 9 for sensitivity to BoNT/A at 1,000 pM were treated with 100 pM BoNT/A in the fashion previously discussed for 96 hours or not treated (control). FIGS. 11E-F depict, respectively, flow cytometry data for the control and the cells treated with 100 pM BoNT/A for 96 hours.

In FIGS. 11A-F, the roughly circular gate highlights the cells the exhibit neither red nor green fluorescence (i.e., cells that do not express the indicator protein), the roughly oval gate highlights the cells that exhibit both red and green fluorescence (i.e., cells that express the indicator protein wherein the indicator protein has not been cleaved), and the quadrilateral gate highlights the cells that exhibit red fluorescence but comparatively decreased green fluorescence (i.e., cells that express the indicator protein wherein the indicator protein has been cleaved).

The cells previously selected for sensitivity to 1 nM BoNT/A (FIG. 11D) exhibited a significantly greater sensitivity to BoNT/A at 100 pM (greater cleavage) than the cells that were not so selected (FIG. 11F).

The cells that were sensitive to BoNT/A at 100 pM after 96 hours of treatment (>2 logs more sensitive than wild type NG108) were selected.

### Example 11—Directed Evolution of Cells

The cells from Example 10 that were sensitive to BoNT/A at 100 pM after 96 hours of treatment were expanded and treated with 10 pM BoNT/A for 96 hours in the fashion previously discussed or not treated (control). After treatment, the cells were washed three times, trypsinized, resuspended in fresh media, and sorted.

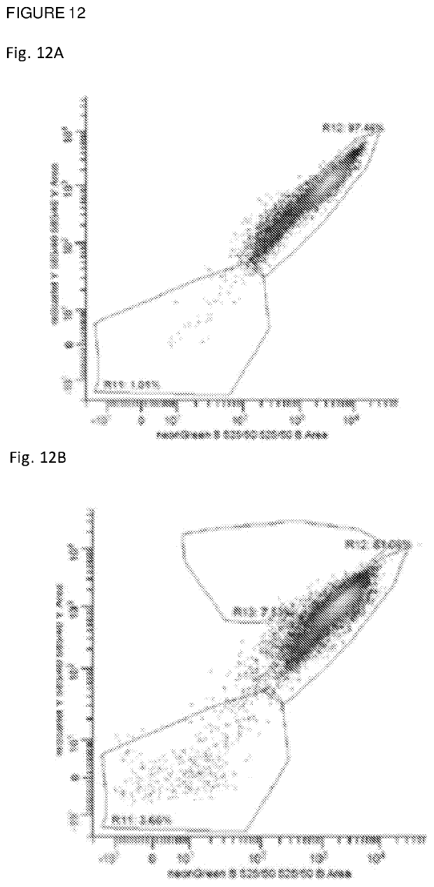

FIGS. 12A-B depict, respectively, flow cytometry data for the control and the cells treated with 10 pM BoNT/A. There was a noticeable shift in the fluorescence of treated cells, albeit not as dramatic as seen with higher concentrations of toxin.

### Example 12—Receptor Construct for BoNT/E

To confer sensitivity to BoNT/E, NG108 cells from Example 2 that were stably transfected with the mScarlet-SNAP25-GeNluc indicator construct were transfected with a plasmid containing a GD3-SV2A-Syt receptor construct (SEQ ID NO: 5) using the transfection procedure described in Example 2. This plasmid was constructed by modifying the plasmid containing the GD2-SV2C-Syt receptor construct using a HiFi Kit (New England Biolabs), oligonucleotides from IDT, and SV2A sequence synthesized by GeneArt.

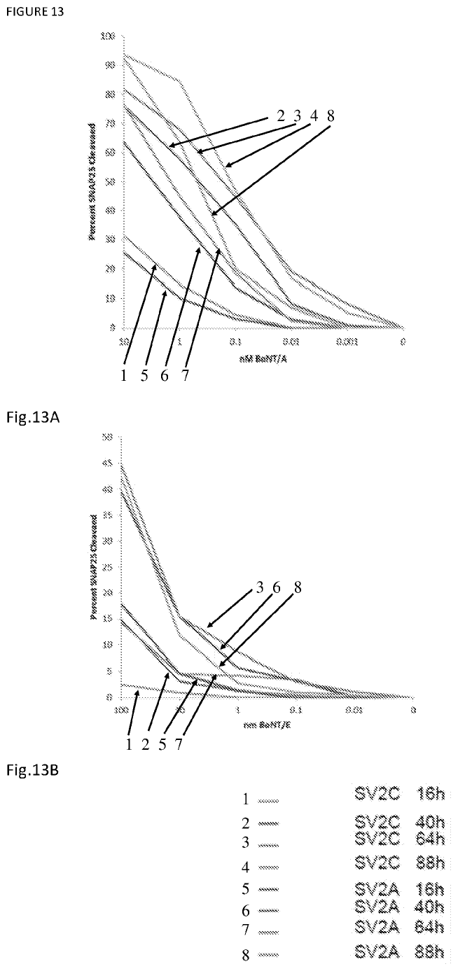

These cells and cells from Example 7 that expressed the GD3-SV2C-Syt receptor construct were cultured in media containing either BoNT/A at 0 (control), 10 nM, 1 nM, 0.1 nM, 0.01 nM, 0.001 nM, or 0 nM (control) BoNT/A or BoNT/E at 100 nM, 10 nM, 1 nM, 0.1 nM, 0.01 nM, or 0 nM (control). The cells were treated for 16, 40, 64, or 88 hours. Following treatment, the cells were lysed and anti-SNAP-25 Western blot was performed using anti-SNAP-25 antibody. Densitometry data from the blot was plotted as a percent of SNAP-25 cleaved (FIG. 13). There was a marked increase in the sensitivity of the cells expressing SV2A to both BoNT/A and BoNT/E as compared to the cells expressing SV2C, confirming the need for SV2A to confer BoNT/E sensitivity.

### SEQUENCE LISTING

### Description of the Sequences

SEQ ID NO: 1 is the nucleotide sequence of the nucleic acid encoding a fusion protein comprising an N-terminal mScarlet label, SNAP-25, a C-terminal NeonGreen label, and a C-terminal luciferase.

SEQ ID NO: 2 is the nucleotide sequence of the nucleic acid encoding a fusion protein comprising an N-terminal mScarlet label, SNAP-25, a C-terminal CFP label, and a C-terminal luciferase.

SEQ ID NO: 3 is the nucleotide sequence of the nucleic acid encoding a fusion protein comprising an N-terminal CFP and the light chain of BoNT/A.

SEQ ID NO: 4 is the nucleotide sequence of the nucleic acid encoding a fusion protein comprising domains having the amino acid sequences for GD3 synthase, SV2C, syntaxin, and aminoglycoside 3′-phosphotransferase. In the fusion protein, each domain is separated from each other by a 2A self-cleaving peptide.

SEQ ID NO: 5 is the nucleotide sequence of the nucleic acid encoding a fusion protein comprising domains having the amino acid sequences for GD3 synthase, SV2A, syntaxin, and aminoglycoside 3′-phosphotransferase. In the fusion protein, each domain is separated from each other by a 2A self-cleaving peptide.

SEQ ID NO: 6 is the amino acid sequence for GD3 synthase.

SEQ ID NO: 7 is the amino acid sequence for a 2A self-cleaving peptide encoded by SEQ ID NOs: 4 and 5.

SEQ ID NO: 8 is the amino acid sequence for SV2A.

SEQ ID NO: 9 is the amino acid sequence for SV2B.

SEQ ID NO: 10 is the amino acid sequence for SV2C.

SEQ ID NO: 11 is the amino acid sequence for the fourth luminal domain of SV2A.

SEQ ID NO: 12 is the amino acid sequence for the fourth luminal domain of SV2B.

SEQ ID NO: 13 is the amino acid sequence for the fourth luminal domain of SV2C.

SEQ ID NO: 14 is the amino acid sequence for synaptotagmin I.

SEQ ID NO: 15 is the amino acid sequence for synaptotagmin II.

SEQ ID NO: 16 is the amino acid sequence for MScarlet.

SEQ ID NO: 17 is the amino acid sequence for NeonGreen.

SEQ ID NO: 18 is the amino acid sequence for CFP.

SEQ ID NO: 19 is the amino acid sequence for SNAP-25.

SEQ ID NO: 20 is the amino acid sequence for aminoglycoside 3′-phosphotransferase (Neo).

SEQ ID NO: 21 is the amino acid sequence for puromycin-N-acetyltransferase (PuroR).

SEQ ID NO: 22 is the amino acid sequence for luciferase.

SEQ ID NO: 1
ATGGTGTCGAAGGGGGAAGCGGTGATCAAGGAGTTCATGA
GGTTTAAAGTGCATATGGAGGGATCTATGAACGGACACGA
GTTTGAGATCGAAGGGGAAGGAGAGGGGCGCCCATACGAA
GGCACCCAGACTGCCAAGCTGAAAGTCACAAAGGGTGGAC
CCTTGCCCTTCTCGTGGGATATTCTGAGCCCGCAATTCAT
GTACGGGTCCCGGGCCTTCACCAAGCACCCTGCTGACATT
CCGGATTACTATAAGCAGAGCTTCCCGGAAGGCTTCAAAT
GGGAGCGAGTGATGAACTTCGAGGATGGAGGCGCCGTGAC
CGTGACTCAGGACACTTCACTGGAAGATGGCACTCTGATC
TACAAGGTCAAGCTGCGGGGCACCAACTTCCCACCGGACG
GACCGGTCATGCAGAAAAAGACCATGGGATGGGAGGCCTC
CACCGAGCGCCTGTACCCCGAAGATGGAGTCCTCAAGGGG
GACATCAAGATGGCCCTGCGGCTCAAGGATGGTGGAAGAT
ACCTGGCTGACTTCAAGACCACGTACAAGGCCAAGAAGCC
AGTCCAGATGCCCGGCGCGTACAATGTGGATCGCAAGCTG
GACATCACTTCCCACAACGAGGACTACACCGTGGTGGAGC
AGTACGAACGGTCCGAGGGTCGGCACTCCACTGGTGGCAT
GGACGAGCTGTACAAAATGGCCGAGGATGCAGACATGAGA
AACGAACTGGAAGAAATGCAGCGGAGAGCAGACCAGCTCG
CGGACGAATCACTGGAATCGACCCGCCGGATGCTTCAACT
GGTCGAGGAATCAAAGGACGCGGGTATCCGGACCCTTGTG
ATGCTGGACGAACAGGGAGAGCAGCTGGAGAGGATCGAAG
AGGGAATGGACCAGATTAACAAGGACATGAAGGAAGCGGA
AAAGAACCTCACCGACCTTGGAAAGTTCTGCGGGTTGTGC
GTGTGTCCGTGCAACAAGCTGAAGTCCTCCGACGCCTACA
AGAAGGCCTGGGGAAACAACCAGGACGGTGTCGTGGCTTC
CCAACCCGCACGGGTGGTGGATGAGCGGGAACAGATGGCG
ATTTCCGGAGGCTTCATTAGACGCGTGACCAACGACGCCC
GCGAAAACGAGATGGACGAAAACCTGGAACAAGTGTCGGG
AATCATCGGAAACTTGAGACACATGGCCCTCGACATGGGC
AACGAAATTGATACACAGAACCGGCAGATTGACCGGATCA
TGGAAAAGGCAGACTCAAACAAGACTCGGATTGACGAAGC
GAACCAGAGGGCCACTAAGATGTTGGGTTCCGGGATGGTG
TCAAAGGGAGAAGAAGACAACATGGCATCACTGCCCGCCA
CCCACGAGCTGCACATCTTCGGTTCCATCAACGGGGTCGA
CTTCGACATGGTCGGCCAGGGAACTGGAAACCCGAATGAC
GGTTATGAAGAACTGAACCTTAAATCAACCAAGGGGGACC
TTCAGTTCTCGCCCTGGATTTTGGTCCCTCACATTGGATA
CGGATTCCATCAGTATCTGCCGTACCCCGACGGAATGAGC
CCGTTCCAGGCTGCCATGGTGGACGGATCGGGATACCAGG
TCCACCGCACCATGCAGTTTGAAGATGGCGCAAGCCTGAC
CGTGAACTACCGGTATACCTACGAGGGCTCACACATCAAG
GGGGAAGCCCAAGTCAAGGGTACCGGCTTCCCGGCCGACG
GACCAGTGATGACCAACTCCTTGACCGCCGCCGACTGGTG
CCGCAGCAAGAAAACTTACCCCAACGATAAGACAATCATC
TCCACTTTCAAGTGGTCCTACACCACGGGCAACGGCAAAC
GCTACCGAAGCACTGCACGGACCACCTACACTTTCGCGAA
GCCTATGGCCGCCAACTACCTGAAGAACCAGCCGATGTAC
GTGTTCAGAAAGACGGAACTCAAGCACTCCAAAACCGAAC
TGAACTTTAAGGAGTGGCAGAAGGCTTTCACTGGATTCGA
GGACTTTGTCGGCGACTGGCGCCAGACTGCCGGCTACAAC
CTGGACCAAGTGCTCGAACAGGGGGGTGTCTCCAGCCTCT
TCCAAAATCTGGGCGTGTCCGTGACCCCGATCCAGCGGAT
CGTGCTCAGCGGGGAAAACGGCCTGAAGATCGATATCCAC
GTCATCATCCCGTACGAGGGACTGAGCGGCGACCAGATGG
GTCAGATCGAAAAGATTTTCAAGGTGGTCTATCCCGTGGA
TGACCACCACTTCAAAGTGATCCTGCATTACGGGACCCTC
GTGATCGACGGCGTCACCCCGAACATGATTGATTACTTCG
GACGGCCTTATGAAGGGATCGCCGTGTTCGACGGCAAAAA
GATCACCGTGACTGGCACCCTGTGGAACGGAAATAAGATC
ATTGACGAGCGGCTGATCAACCCAGACGGGTCGCTGCTGT
TCCGCGTGACCATCAACGGAGTGACCGGCTGGCGGCTGTG
CGAGCGCATCCTCGCCTGATAG
SEQ ID NO: 2
ATGGTGTCGAAGGGGGAAGCGGTGATCAAGGAGTTCATGA
GGTTTAAAGTGCATATGGAGGGATCTATGAACGGACACGA
GTTTGAGATCGAAGGGGAAGGAGAGGGGCGCCCATACGAA
GGCACCCAGACTGCCAAGCTGAAAGTCACAAAGGGTGGAC
CCTTGCCCTTCTCGTGGGATATTCTGAGCCCGCAATTCAT
GTACGGGTCCCGGGCCTTCACCAAGCACCCTGCTGACATT
CCGGATTACTATAAGCAGAGCTTCCCGGAAGGCTTCAAAT
GGGAGCGAGTGATGAACTTCGAGGATGGAGGCGCCGTGAC
CGTGACTCAGGACACTTCACTGGAAGATGGCACTCTGATC
TACAAGGTCAAGCTGCGGGGCACCAACTTCCCACCGGACG
GACCGGTCATGCAGAAAAAGACCATGGGATGGGAGGCCTC
CACCGAGCGCCTGTACCCCGAAGATGGAGTCCTCAAGGGG
GACATCAAGATGGCCCTGCGGCTCAAGGATGGTGGAAGAT
ACCTGGCTGACTTCAAGACCACGTACAAGGCCAAGAAGCC
AGTCCAGATGCCCGGCGCGTACAATGTGGATCGCAAGCTG
GACATCACTTCCCACAACGAGGACTACACCGTGGTGGAGC
AGTACGAACGGTCCGAGGGTCGGCACTCCACTGGTGGCAT
GGACGAGCTGTACAAAATGGCCGAGGATGCAGACATGAGA
AACGAACTGGAAGAAATGCAGCGGAGAGCAGACCAGCTCG
CGGACGAATCACTGGAATCGACCCGCCGGATGCTTCAACT
GGTCGAGGAATCAAAGGACGCGGGTATCCGGACCCTTGTG
ATGCTGGACGAACAGGGAGAGCAGCTGGAGAGGATCGAAG
AGGGAATGGACCAGATTAACAAGGACATGAAGGAAGCGGA
AAAGAACCTCACCGACCTTGGAAAGTTCTGCGGGTTGTGC
GTGTGTCCGTGCAACAAGCTGAAGTCCTCCGACGCCTACA
AGAAGGCCTGGGGAAACAACCAGGACGGTGTCGTGGCTTC
CCAACCCGCACGGGTGGTGGATGAGCGGGAACAGATGGCG
ATTTCCGGAGGCTTCATTAGACGCGTGACCAACGACGCCC
GCGAAAACGAGATGGACGAAAACCTGGAACAAGTGTCGGG
AATCATCGGAAACTTGAGACACATGGCCCTCGACATGGGC
AACGAAATTGATACACAGAACCGGCAGATTGACCGGATCA
TGGAAAAGGCAGACTCAAACAAGACTCGGATTGACGAAGC
GAACCAGAGGGCCACTAAGATGTTGGGTTCCGGGATGGTG
TCAAAGGGAGAAGAACTGTTCACTGGAGTGGTGCCCATCC
TGGTGGAGCTGGATGGCGATGTGAACGGCCATAAATTCTC
AGTCAGCGGAGAGGGAGAGGGCGATGCGACTTACGGAAAG
CTGACTTTGAAGTTTATCTGCACTACCGGAAAGCTGCCTG
TGCCATGGCCTACCCTCGTGACCACCCTGTCCTGGGGCGT
CCAATGTTTCGCACGCTACCCTGACCATATGAAGCAGCAC
GACTTCTTCAAGTCCGCCATGCCCGAGGGCTACGTGCAGG
AACGCACCATCTTCTTCAAGGACGACGGGAACTACAAAAC
CAGGGCTGAAGTGAAGTTCGAGGGAGACACCCTGGTCAAT
CGGATTGAATTGAAGGGAATCGATTTCAAGGAAGATGGAA
ACATCCTGGGACATAAGCTTGAGTACAACTACTTCTCCGA
CAACGTGTACATCACGGCCGATAAGCAGAAGAACGGAATC
AAAGCTAACTTCAAGATTCGGCACAACATTGAGGACGGCG
GCGTCCAGCTGGCGGACCATTATCAGCAGAATACCCCTAT
TGGGGATGGACCGGTGCTGCTCCCGGACAACCATTACCTG
TCCACCCAATCTAAGCTGAGCAAGGACCCAAACGAGAAGC
GCGATCACATGGTGCTGCTCGAGTTCGTGACTGCCGCCGG
GCTTCACACACTTGAGGACTTTGTCGGCGACTGGCGCCAG
ACTGCCGGCTACAACCTGGACCAAGTGCTCGAACAGGGGG
GTGTCTCCAGCCTCTTCCAAAATCTGGGCGTGTCCGTGAC
CCCGATCCAGCGGATCGTGCTCAGCGGGGAAAACGGCCTG
AAGATCGATATCCACGTCATCATCCCGTACGAGGGACTGA
GCGGCGACCAGATGGGTCAGATCGAAAAGATTTTCAAGGT
GGTCTATCCCGTGGATGACCACCACTTCAAAGTGATCCTG
CATTACGGGACCCTCGTGATCGACGGCGTCACCCCGAACA
TGATTGATTACTTCGGACGGCCTTATGAAGGGATCGCCGT
GTTCGACGGCAAAAAGATCACCGTGACTGGCACCCTGTGG
AACGGAAATAAGATCATTGACGAGCGGCTGATCAACCCAG
ACGGGTCGCTGCTGTTCCGCGTGACCATCAACGGAGTGAC
CGGCTGGCGGCTGTGCGAGCGCATCCTCGCCTGATAG
SEQ ID NO: 3
ATGACCATGGATGAGCAGCAATCGCAGGCTGTAGCCCCGG
TATATGTCGGTGGTATGGATGAGAAAACGACTGGGTGGCG
GGGTGGACACGTCGTCGAGGGCCTGGCAGGCGAACTTGAA
CAACTGCGGGCTCGCTTGGAGCACCACCCGCAAGGACAGC
GCGAGCCGTCCATGGTGTCAAAGGGGGAGGAACTGTTTAC
TGGGGTCGTCCCTATCTTGGTGGAACTCGACGGGGATGTG
AACGGACACAAGTTTTCGGTATCCGGGGAAGGCGAGGGGG
ATGCCACgTATGGAAAGCTCACACTTAAGTTCATCTGCACG
ACAGGGAAGCTCCCAGTGCCTTGGCCCACGTTGGTGACTAC
GCTCACATGGGGTGTCCAGTGCTTCGCACGGTATCCCGACC
AcATGAAGCAGCATGATTTCTTTAAGTCAGCCATGCCGGAG
GGATATGTACAAGAAAGGACCATCTTCTTCAAAGATGACGG
TAACTACAAGACCAGAGCCGAGGTAAAGTTTGAAGGCGAC
ACTCTCGTGAACAGGATTGAGCTGAAGGGAATTGATTTCA
AAGAGGATGGGAACATCCTTGGTCACAAATTGGAGTACAA
TGCCATTTCGGATAACGTGTACATTACAGCGGATAAGCAG
AAGAATGGGATCAAAGCGAATTTCAAAATCAGGCATAACA
TCGAGGACGGGTCGGTGCAGCTCGCCGACCATTACCAGCA
GAATACGCCCATCGGAGATGGACCCGTACTTCTGCCCGAC
AATCATTATCTGTCAACGCAATCAGCGCTTAGCAAAGATC
CCAATGAGAAAAGGGACCACATGGTGCTCCTCGAATTtGT
GACGGCAGCGGGAATTACCCTCGGGATGGACGAACTGT
ACAAAAGCGGGTTGAGACTCGAGCGCTGAACTCGAGATGC
CTTTTGTCAACAAGCAGTTTAACTATAAGGATCCCGTGAA
TGGTGTGGACATTGCCTACATCAAGATTCCAAACGCTGGA
CAAATGCAGCCCGTCAAGGCTTTCAAAATTCACAACAAGA
TCTGGGTGATCCCGGAGAGGGACACCTTTACCAATCCAGA
AGAGGGCGACCTTAACCCTCCGCCAGAGGCCAAACAGGTG
CCCGTGAGCTATTACGACTCAACTTATCTCTCCACCGACA
ACGAAAAGGACAATTACCTCAAGGGAGTCACCAAGCTGTT
CGAACGGATCTACTCTACCGATCTCGGCAGGATGCTCCTG
ACTTCTATCGTGCGGGGCATCCCCTTCTGGGGTGGGAGCA
CCATTGACACCGAACTGAAGGTGATTGATACCAATTGCAT
CAACGTCATCCAGCCAGACGGTTCCTACCGGTCTGAGGAG
CTCAATCTTGTGATTATTGGCCCGTCAGCTGATATCATCC
AGTTCGAATGCAAGTCTTTCGGACACGACGTGCTTAATCT
CACCCGCAATGGTTACGGAAGCACCCAGTACATCAGATTC
TCTCCGGACTTTACTTTCGGATTTGAAGAGTCACTGGAAG
TCGACACCAATCCTCTGCTCGGAGCCGGAAAGTTCGCCAC
CGACCCTGCAGTGACCCTTGCTCACGAGCTGATTCATGCA
GAGCATCGCCTGTACGGGATCGCCATCAATCCTAACCGCG
TGTTTAAGGTCAATACCAACGCTTACTATGAAATGAGCGG
ACTGGAGGTGTCCTTCGAGGAACTGCGCACCTTCGGAGGT
CATGACGCTAAGTTCATCGACTCACTGCAAGAGAATGAGT
TCCGGCTGTACTATTACAACAAGTTTAAGGATGTCGCCTC
AACTCTGAACAAGGCCAAAAGCATCATCGGCACCACCGCC
AGCCTGCAATACATGAAAAACGTGTTCAAGGAAAAGTACC
TTCTTAGCGAAGATACTTCCGGGAAGTTTTCAGTCGACAA
ACTGAAGTTCGACAAGCTGTACAAGATGCTCACCGAAATC
TACACCGAGGACAATTTTGTGAACTTCTTCAAAGTGATTA
ACAGAAAGACCTATCTGAACTTCGACAAAGCCGTGTTCCG
GATTAACATTGTGCCCGATGAGAACTACACTATCAAGGAC
GGGTTCAACCTTAAGGGTGCAAATCTTTCAACTAATTTCA
ACGGACAGAATACTGAGATCAATTCAAGGAACTTCACTAG
ACTCAAGAATTTCACTGGGCTTTTCGAGTTCTATAAGCTG
CTGTGTGTCCGCGGAATTATCCCCTTCAAGTGAAGCTTCG
TCAATGA
SEQ ID NO: 4
ATGTCCCCATGTGGACGAGCGCGCAGACAGACCTCAAGAG
GAGCGATGGCCGTGCTGGCCTGGAAGTTCCCGAGGACTC
GCCTGCCCATGGGAGCCTCTGCTCTGTGTGTGGTCGTGC
TGTGTTGGCTGTACATCTTCCCGGTGTACCGGCTGCCTAA
CGAAAAGGAAATTGTGCAGGGCGTGCTCCAGCAGGGGACCGC
TTGGCGGCGCAACCAGACCGCTGCGAGGGCTTTTCGGAAGC
AGATGGAAGATTGTTGCGACCCCGCCCATCTTTTCGCGATG
ACCAAGATGAACAGCCCGATGGGAAAGTCCATGTGGTACGA
CGGAGAGTTCCTGTATTCCTTCACCATTGACAACAGCACTT
ACTCACTGTTCCCGCAAGCCACCCCCTTCCAACTGCCGCT
TAAGAAGTGCGCCGTCGTGGGGAACGGCGGCATCCTCAAG
AAGTCCGGATGCGGGCGCCAGATTGATGAAGCCAACTTCG
TGATGCGGTGCAATCTCCCGCCACTCTCGTCCGAGTACAC
CAAGGACGTGGGGTCAAAGTCGCAGCTCGTCACCGCCAAC
CCTTCGATCATCAGACAACGGTTCCAGAACCTTCTGTGGA
GCCGGAAAACATTTGTGGATAACATGAAGATCTACAACCA
TTCCTACATCTATATGCCTGCCTTCTCCATGAAAACTGGA
ACCGAACCCTCCCTGAGAGTGTACTACACCCTGTCCGACG
TGGGCGCAAACCAGACCGTCCTTTTCGCCAACCCCAACTT
CCTGCGCTCCATCGGAAAGTTCTGGAAGTCCAGAGGCATT
CACGCGAAACGCTTGTCCACTGGATTGTTCTTGGTGTCCG
CCGCTCTGGGCCTGTGCGAGGAAGTGGCCATATACGGATT
CTGGCCTTTCTCCGTCAACATGCACGAGCAGCCCATCTCC
CACCATTATTACGACAATGTCCTGCCTTTCTCGGGATTTC
ACGCGATGCCCGAGGAGTTCTTGCAACTGTGGTACCTTCA
CAAGATCGGTGCCCTGCGGATGCAGCTGGACCCTTGCGAG
GACACCTCGCTGCAACCCACCTCGGAGCAGAAACTCATTT
CCGAAGAGGATCTGAACGGGGAGCAGAAGCTCATCTCCGA
GGAGGACCTGAACGGAGAACAGAAGCTGATTAGCGAAGAG
GACCTGGGCAGCGGTGCCACCAATTTTTCTCTGCTCAAGC
AGGCCGGAGATGTGGAAGAGAACCCGGGTCCCATGGAGGA
CTCCTACAAAGATCGGACTTCTCTGATGAAGGGAGCCAAG
GACATCGCCAGGGAAGTGAAGAAGCAAACCGTCAAGAAGG
TCAACCAGGCCGTGGACAGAGCCCAGGACGAGTACACCCA
GCGGTCGTACTCGCGGTTCCAGGATGAAGAGGATGACGAC
GACTACTACCCTGCCGGCGAAACCTATAATGGGGAAGCCA
ACGATGACGAAGGCTCCAGCGAAGCCACTGAAGGACACGA
CGAGGACGACGAAATCTACGAAGGAGAATACCAGGGCATC
CCTTCGATGAATCAGGCCAAAGATTCAATTGTGTCAGTGG
GACAGCCTAAGGGCGACGAGTACAAGGACCGGAGAGAGCT
CGAAAGCGAGCGGAGGGCCGACGAAGAGGAACTGGCACAA
CAGTACGAGCTGATCATCCAGGAGTGTGGGCACGGCCGGT
TCCAGTGGGCGCTGTTCTTCGTGCTCGGAATGGCACTGAT
GGCCGACGGCGTGGAAGTGTTCGTGGTCGGATTCGTGCTG
CCCTCGGCCGAAACCGACCTCTGCATTCCCAACTCCGGCT
CGGGATGGCTGGGGTCCATCGTGTACCTGGGAATGATGGT
CGGCGCCTTCTTCTGGGGTGGCCTGGCAGACAAGGTCGGC
CGGAAGCAGTCCCTCTTGATCTGCATGAGCGTCAACGGAT
TTTTCGCCTTCCTGTCATCATTCGTGCAAGGTTACGGGTT
CTTCCTTTTCTGCCGCCTGCTGTCCGGCTTTGGGATCGGC
GGGGCTATTCCGACTGTGTTCTCCTACTTTGCCGAAGTGC
TGGCTCGCGAAAAACGGGGCGAACACCTTTCCTGGCTGTG
TATGTTCTGGATGATCGGCGGCATCTACGCCTCGGCCATG
GCCTGGGCTATTATCCCGCATTATGGGTGGTCCTTCTCAA
TGGGAAGCGCATACCAGTTCCATTCGTGGCGGGTGTTCGT
GATCGTGTGCGCCCTCCCGTGTGTGTCCTCCGTGGTGGCT
CTGACATTCATGCCGGAGTCACCTCGGTTCTTGTTGGAAG
TCGGGAAGCACGACGAAGCCTGGATGATTCTGAAGCTGAT
CCACGACACTAATATGCGGGCCCGGGGACAGCCTGAGAAA
GTGTTCACCGTCAACAAGATTAAGACCCCGAAGCAAATCG
ATGAACTGATTGAAATTGAGTCCGACACCGGAACTTGGTA
CCGCCGGTGCTTCGTGCGGATTCGCACCGAGCTGTACGGA
ATCTGGCTCACCTTCATGCGCTGCTTCAACTACCCCGTGC
GCGACAACACCATCAAGCTGACCATCGTGTGGTTCACTCT
GTCTTTCGGCTAGTATGGGCTGTCAGTGTGGTTCCCGGAT
GTCATCAAGCCGCTCCAATCCGATGAATACGCCCTGCTGA
CCCGCAATGTGGAGAGAGACAAATACGCCAACTTCACCAT
CAATTTCACCATGGAAAACCAGATTCACACCGGAATGGAG
TACGACAATGGACGATTCATCGGAGTGAAGTTCAAGAGCG
TGACCTTCAAGGACTCGGTGTTCAAGTCCTGTACCTTCGA
GGACGTGACCAGCGTGAACACTTATTTTAAGAATTGCACCT
TCATCGATACTGTGTTCGATAACACCGACTTCGAGC
CCTATAAGTTCATTGACTCGGAGTTCAAGAACTGTTCATT
CTTCCACAACAAGACTGGTTGCCAGATCACCTTCGATGACGA
CTACAGCGCCTACTGGATCTACTTTGTGAACTTTTTGGGAA
CTCTCGCAGTGCTTCCTGGCAACATTGTGTCCGCACTCCTG
ATGGATCGGATTGGCAGGCTCACGATGCTTG
GGGGGTCCATGGTCCTCTCCGGGATCTCGTGCTTCTTCCT
GTGGTTCGGCACCTCGGAGTCCATGATGATCGGAATGTTG
TGCCTGTACAACGGTCTGACCATGTGCGCCTGGAACAGCC
TCGACGTGGTCACCGTCGAGCTGTA
TCCTACCGACCGGCGCGCGACAGGCTTCGGATTCCTGAAC
GCACTGTGCAAGGCAGCCGCGGTCCTGGGAAATCTGATCT
TTGGTTCGCTGGTGTCCATCACTAAGAGCATCCCTATTCT
GCTCGCCTCCACGGTGCTCGTGTGTGGTGGCCTGGTCGGG
CTGTGCCTGCCCGACACTCGCACCCAAGTGCTCATGGACT
ACAAGGATGACGATGATAAGGGAGACTACAAGGACGATGA
CGACAAGGGGGATTACAAGGACGACGATGACAAAGGAAGC
GGCGCCACTAACTTTTCCCTGCTGAAGCAGGCCGGGGACG
TCGAAGAAAACCCCGGGCCAATGCGCAACATTTTCAAGCG
GAATCAGGAGCCTATCGTGGCCCCGGCCACCACTACCGCC
ACTATGCCTATTGGACCTGTCGACAACTCCACGGAATCAG
GCGGCGCCGGCGAATCCCAAGAGGACATGTTCGCCAAGCT
GAAGGAGAAACTGTTCAACGAAATCAACAAGATTCCCCTC
CCGCCGTGGGCCCTGATCGCTATCGCTGTCGTCGCCGGAC
TGCTGCTGCTTACTTGCTGCTTCTGCATTTGCAAGAAGTG
TTGTTGCAAGAAAAAGAAAAACAAGAAGGAAAAGGGGAAG
GGAATGAAGAACGCCATGAATATGAAGGACATGAAGGGCG
GACAGGATGATGATGATGCTGAAACTGGGCTGACTGAGGG
CGAAGGAGAGGGCGAAGAGGAGAAGGAACCTGAGAACCTG
GGAAAGCTCCAATTCTCCCTGGATTACGACTTCCAAGCCA
ACCAGCTGACTGTGGGAGTGTTGCAAGCCGCCGAGCTGCC
AGCCCTGGACATGGGCGGCACCTCCGACCCCTATGTGAAG
GTGTTCTTGCTGCCTGACAAGAAGAAGAAGTACGAAACCA
AGGTGCACCGCAAGACCCTGAACCCCGCTTTCAACGAAAC
CTTCACTTTCAAAGTGCCCTACCAAGAGCTCGGGGGAAAG
ACTCTCGTGATGGCGATCTACGACTTCGACCGGTTCAGCA
AGCACGATATCATCGGGGAGGTCAAGGTCCCGATGAACAC
CGTGGACCTTGGCCAACCGATTGAAGAATGGCGCGATCTC
CAGGGTGGCGAAAAGGAGGAGCCCGAGAAACTGGGTGACA
TCTGTACATCACTGCGCTACGTGCCGACCGCCGGGAAGCT
CACTGTCTGCATCCTGGAGGCCAAGAACCTGAAGAAAATG
GACGTGGGCGGGCTCTCCGACCCTTACGTGAAGATCCACC
TGATGCAGAACGGAAAGCGGCTGAAGAAGAAGAAAACCAC
TGTGAAGAAAAAGACTCTGAACCCCTACTTCAACGAGTCG
TTCTCCTTCGAAATCCCGTTTGAGCAAATCCAGAAGGTCC
AAGTGGTCGTGACTGTGCTTGACTACGACAAGCTCGGAAA
GAACGAGGCCATTGGCAAAATCTTCGTGGGATCGAACGCA
ACTGGCACCGAGCTGAGACACTGGTCTGACATGCTCGCCA
ACCCAAGGCGGCCGATTGCTCAGTGGCACTCCTTGAAACC
TGAGGAAGAAGTGGATGCCCTTCTTGGAAAGAACAAGATG
TACCCCTACGACGTCCCTGATTACGCGGGATACCCGTACG
ATGTGCCTGACTATGCCGGCTACCCGTACGATGTGCCAGA
CTACGCTGGCTCCGGAGCCACGAACTTTTCGCTGCTGAAA
CAGGCCGGCGACGTGGAAGAAAATCCCG
GTCCAATGATTGAACAAGATGGATTGCACGCTGGTTCTCC
GGCCGCTTGGGTGGAGAGGCTATTCGGCTATGACTGGGCA
CAACAGACAATCGGCTGCTCTGATGCCGCCGTGTTCCGGC
TGTCAGCGCAGGGGCGCCCGGTTCTTTTTGTCAAGACCGA
CCTGTCCGGTGCCCTGAATGAACTGCAGGACGAGGCAGCG
CGGCTATCGTGGCTGGCCACGACGGGCGTTCCTTGCGCAG
CTGTGCTCGACGTTGTCACTGAAGCGGGAAGGGACTGGCT
GCTATTGGGCGAAGTGCCGGGGCAGGATCTCCTGTCATCT
CACCTTGCTCCTGCCGAGAAAGTATCCATCATGGCTGATG
CAATGCGGCGGCTGCATACGCTTGATCCGGCTACATGCCC
ATTCGACCACCAAGCGAAACATCGCATCGAGCGAGCACGT
ACTCGGATGGAAGCCGGTCTTGTCGATCAGGATGATCTGG
ACGAGGAACATCAGGGGCTCGCGCCAGCCGAACTGTTCGC
CAGGCTCAAGGCGCGCATGCCCGACGGCGAGGATCTCGTC
GTGACCCATGGCGATGCCTGCTTGCCGAATATCATGGTGG
AAAATGGCCGCTTTTCTGGATTCATCGACTGTGGCCGGCT
GGGTGTGGCGGACCGCTATCAGGACATAGCGTTGGCTACC
CGTGATATTGCTGAGGAACTTGGCGGCGAATGGGCTGACC
GCTTCCTCGTGCTTTACGGTATCGCCGCTCCCGATTCGCA
GCGCATCGCCTTCTATCGCCTTCTTGACGAGTTCTTCTGA
TAG
SEQ ID NO: 5
ATGTCCCCATGTGGACGAGCGCGCAGACAGACCTCAAGAG
GAGCGATGGCCGTGCTGGCCTGGAAGTTCCCGAGGACTCG
CCTGCCCATGGGAGCCTCTGCTCTGTGTGTGGTCGTGCTG
TGTTGGCTGTACATCTTCCCGGTGTACCGGCTGCCTAACG
AAAAGGAAATTGTGCAGGGCGTGCTCCAGCAGGGGACCGC
TTGGCGGCGCAACCAGACCGCTGCGAGGGCTTTTCGGAAG
CAGATGGAAGATTGTTGCGACCCCGCCCATCTTTTCGCGA
TGACCAAGATGAACAGCCCGATGGGAAAGTCCATGTGGTA
CGACGGAGAGTTCCTGTATTCCTTCACCATTGACAACAGC
ACTTACTCACTGTTCCCGCAAGCCACCCCCTTCCAACTGC
CGCTTAAGAAGTGCGCCGTCGTGGGGAACGGCGGCATCCT
CAAGAAGTCCGGATGCGGGCGCCAGATTGATGAAGCCAAC
TTCGTGATGCGGTGCAATCTCCCGCCACTCTCGTCCGAGT
ACACCAAGGACGTGGGGTCAAAGTCGCAGCTCGTCACCGC
CAACCCTTCGATCATCAGACAACGGTTCCAGAACCTTCTG
TGGAGCCGGAAAACATTTGTGGATAACATGAAGATCTACA
ACCATTCCTACATCTATATGCCTGCCTTCTCCATGAAAAC
TGGAACCGAACCCTCCCTGAGAGTGTACTACACCCTGTCC
GACGTGGGCGCAAACCAGACCGTCCTTTTCGCCAACCCCA
ACTTCCTGCGCTCCATCGGAAAGTTCTGGAAGTCCAGAGG
CATTCACGCGAAACGCTTGTCCACTGGATTGTTCTTGGTG
TCCGCCGCTCTGGGCCTGTGCGAGGAAGTGGCCATATACG
GATTCTGGCCTTTCTCCGTCAACATGCACGAGCAGCCCAT
CTCCCACCATTATTACGACAATGTCCTGCCTTTCTCGGGA
TTTCACGCGATGCCCGAGGAGTTCTTGCAACTGTGGTACC
TTCACAAGATCGGTGCCCTGCGGATGCAGCTGGACCCTTG
CGAGGACACCTCGCTGCAACCCACCTCGGAGCAGAAACTC
ATTTCCGAAGAGGATCTGAACGGGGAGCAGAAGCTCATCT
CCGAGGAGGACCTGAACGGAGAACAGAAGCTGATTAGCGA
AGAGGACCTGGGCAGCGGTGCCACCAATTTTTCTCTGCTC
AAGCAGGCCGGAGATGTGGAAGAGAACCCGGGTCCCATGG
AGGACTCCTACAAAGATCGGACTTCTCTGATGAAGGGAGC
CAAGGACATCGCCAGGGAAGTGAAGAAGCAAACCGTCAAG
AAGGTCAACCAGGCCGTGGACAGAGCCCAGGACGAGTACA
CCCAGCGGTCGTACTCGCGGTTCCAGGATGAAGAGGATGA
CGACGACTACTACCCTGCCGGCGAAACCTATAATGGGGAA
GCCAACGATGACGAAGGCTCCAGCGAAGCCACTGAAGGAC
ACGACGAGGACGACGAAATCTACGAAGGAGAATACCAGGG
CATCCCTTCGATGAATCAGGCCAAAGATTCAATTGTGTCA
GTGGGACAGCCTAAGGGCGACGAGTACAAGGACCGGAGAG
AGCTCGAAAGCGAGCGGAGGGCCGACGAAGAGGAACTGGC
ACAACAGTACGAGCTGATCATCCAGGAGTGTGGGCACGGC
CGGTTCCAGTGGGCGCTGTTCTTCGTGCTCGGAATGGCAC
TGATGGCCGACGGCGTGGAAGTGTTCGTGGTCGGATTCGT
GCTGCCCTCGGCCGAAACCGACCTCTGCATTCCCAACTCC
GGCTCGGGATGGCTGGGGTCCATCGTGTACCTGGGAATGA
TGGTCGGCGCCTTCTTCTGGGGTGGCCTGGCAGACAAGGT
CGGCCGGAAGCAGTCCCTCTTGATCTGCATGAGCGTCAAC
GGATTTTTCGCCTTCCTGTCATCATTCGTGCAAGGTTACG
GGTTCTTCCTTTTCTGCCGCCTGCTGTCCGGCTTTGGGAT
CGGCGGGGCTATTCCGACTGTGTTCTCCTACTTTGCCGAA
GTGCTGGCTCGCGAAAAACGGGGCGAACACCTTTCCTGGC
TGTGTATGTTCTGGATGATCGGCGGCATCTACGCCTCGGC
CATGGCCTGGGCTATTATCCCGCATTATGGGTGGTCCTTC
TCAATGGGAAGCGCATACCAGTTCCATTCGTGGCGGGTGT
TCGTGATCGTGTGCGCCCTCCCGTGTGTGTCCTCCGTGGT
GGCTCTGACATTCATGCCGGAGTCACCTCGGTTCTTGTTG
GAAGTCGGGAAGCACGACGAAGCCTGGATGATTCTGAAGC
TGATCCACGACACTAATATGCGGGCCCGGGGACAGCCTGA
GAAAGTGTTCACCGTCAACAAGATTAAGACCCCGAAGCAA
ATCGATGAACTGATTGAAATTGAGTCCGACACCGGAACTT
GGTACCGCCGGTGCTTCGTGCGGATTCGCACCGAGCTGTA
CGGAATCTGGCTCACCTTCATGCGCTGCTTCAACTACCCC
GTGCGCGACAACACCATCAAGCTGACCATCGTGTGGTTCA
CTCTGTCTTTCGGCTACTATGGGCTGTCAGTGTGGTTCCC
GGATGTCATCAAGCCGCTCCAATCCGATGAATACGCCCTG
CTGACCCGCAATGTGGAGAGAGACAAATACGCCAACTTCA
CCATCAATTTCACCATGGAAAACCAGATTCACACCGGAAT
GGAGTACGACAATGGACGATTCATCGGAGTGAAGTTCAAG
AGCGTGACCTTCAAGGACTCGGTGTTCAAGTCCTGTACCT
TCGAGGACGTGACCAGCGTGAACACTTATTTTAAGAATTG
CACCTTCATCGATACTGTGTTCGATAACACCGACTTCGAG
CCCTATAAGTTCATTGACTCGGAGTTCAAGAACTGTTCAT
TCTTCCACAACAAGACTGGTTGCCAGATCACCTTCGATGA
CGACTACAGCGCCTACTGGATCTACTTTGTGAACTTTTTG
GGAACTCTCGCAGTGCTTCCTGGC
AACATTGTGTCCGCACTCCTGATGGATCGGATTGGCAGGC
TCACGATGCTTGGGGGGTCCATGGTCCTCTCCGGGATCTC
GTGCTTCTTCCTGTGGTTCGGCACCTCGGAGTCCATGATG
ATCGGAATGTTGTGCCTGTACAACGGTCTGACCATCAGCG
CCTGGAACAGCCTCGACGTGGTCACCGTCGAGCTGTATCC
TACCGACCGGCGCGCGACAGGCTTCGGATTCCTGAACGCA
CTGTGCAAGGCAGCCGCGGTCCTGGGAAATCTGATCTTTG
GTTCGCTGGTGTCCATCACTAAGAGCATCCCTATTCTGCT
CGCCTCCACGGTGCTCGTGTGTGGTGGCCTGGTCGGGCTG
TGCCTGCCCGACACTCGCACCCAAGTGCTCATGGACTACA
AGGATGACGATGATAAGGGAGACTACAAGGACGATGACGA
CAAGGGGGATTACAAGGACGACGATGACAAAGGAAGCGGC
GCCACTAACTTTTCCCTGCTGAAGCAGGCCGGGGACGTCG
AAGAAAACCCCGGGCCAATGCGCAACATTTTCAAGCGGAA
TCAGGAGCCTATCGTGGCCCCGGCCACCACTACCGCCACT
ATGCCTATTGGACCTGTCGACAACTCCACGGAATCAGGCG
GCGCCGGCGAATCCCAAGAGGAGTTGTTCGCCAAGCTGAA
GGAGAAACTGTTCAACGAAATCAA
CAAGATTCCCCTCCCGCCGTGGGCCCTGATCGCTATCGCT
GTCGTCGCCGGACTGCTGCTGCTTACTTGCTGCTTCTGCA
TTTGCAAGAAGTGTTGTTGCAAGAAAAAGAAAAACAAGAA
GGAAAAGGGGAAGGGAATGAAGAACGCCATGAATATGAAG
GACATGAAGGGCGGACAGGATGATGATGATGCTGAAACTG
GGCTGACTGAGGGCGAAGGAGAGGGCGAAGAGGAGAAGGA
ACCTGAGAACCTGGGAAAGCTCCAATTCTCCCTGGATTAC
GACTTCCAAGCCAACCAGCTGACTGTGGGAGTGTTGCAAG
CCGCCGAGCTGCCAGCCCTGGACATGGGCGGCACCTCCGA
CCCCTATGTGAAGGTGTTCTTGCTGCCTGACAAGAAGAAG
AAGTACGAAACCAAGGTGCACCGCAAGACCCTGAACCCCG
CTTTCAACGAAACCTTCACTTTCAAAGTGCCCTACCAAGA
GCTCGGGGGAAAGACTCTCGTGATGGCGATCTACGACTTC
GACCGGTTCAGCAAGCACGATATCATCGGGGAGGTCAAGG
TCCCGATGAACACCGTGGACCTTGGCCAACCGATTGAAGA
ATGGCGCGATCTCCAGGGTGGCGAAAAGGAGGAGCCCGAG
AAACTGGGTGACATCTGTACATCACTGCGCTACGTGCCGA
CCGCCGGGAAGCTCACTGTCTGCATCCTGGAGGCCAAGAA
CCTGAAGAAAATGGACGTGGGCGGGCTCTCCGACCCTTAC
GTGAAGATCCACCTGATGCAGAACGGAAAGCGGCTGAAGA
AGAAGAAAACCACTGTGAAGAAAAAGACTCTGAACCCCTA
CTTCAACGAGTCGTTCTCCTTCGAAATCCCGTTTGAGCAA
ATCCAGAAGGTCCAAGTGGTCGTGACTGTGCTTGACTACG
ACAAGCTCGGAAAGAACGAGGCCATTGGCAAAATCTTCGT
GGGATCGAACGCAACTGGCACCGAGCTGAGACACTGGTCT
GACATGCTCGCCAACCCAAGGCGGCCGATTGCTCAGTGGC
ACTCCTTGAAACCTGAGGAAGAAGTGGATGCCCTTCTTGG
AAAGAACAAGATGTACCCCTACGACGTCCCTGATTACGCG
GGATACCCGTACGATGTGCCTGACTATGCCGGCTACCCGT
ACGATGTGCCAGACTACGCTGGCTCCGGAGCCACGAACTT
TTCGCTGCTGAAACAGGCCGGCGACGTGGAAGAAAATCCC
GGTCCAATGATTGAACAAGATGGATTGCACGCTGGTTCTC
CGGCCGCTTGGGTGGAGAGGCTATTCGGCTATGACTGGGC
ACAACAGACAATCGGCTGCTCTGATGCCGCCGTGTTCCGG
CTGTCAGCGCAACTGCAGGACGAGGCAGCGCGGCTATCGT
GGCTGGCCACGACGGGCGTTCCTTGCGCAGCTGTGCTCGA
CGTTGTCACTGAAGCGGGAAGGGACTGGCTGCTATTGGGC
GAAGTGCCGGGGCAGGATCTCCTGTCATCTCACCTTGCTC
CTGCCGAGAAAGTATCCATCATGGCTGATGCAATGCGGCG
GCTGCATACGCTTGATCCGGCTACATGCCCATTCGACCAC
CAAGCGAAACATCGCATCGAGCGAGCACGTACTCGGATGG
AAGCCGGTCTTGTCGATCAGGATGATCTGGACGAGGAACA
TCAGGGGCTCGCGCCAGCCGAACTGTTCGCCAGGCTCAAG
GCGCGCATGCCCGACGGCGAGGATCTCGTCGTGACCCATG
GCGATGCCTGCTTGCCGAATATCATGGTGGAAAATGGCCG
CTTTTCTGGATTCATCGACTGTGGCCGGCTGGGTGTGGCG
GACCGCTATCAGGACATAGCGTTGGCTACCCGTGATATT
GCTGAGGAACTTGGCGGCGAATGGGCTGACCGCTTCCT
CGTGCTTTACGGTATCGCCGCTCCCGATTCGCAGCGCAT
CGCCTTCTATCGCCTTCTTGACGAGTTCTTCTGATAG
SEQ ID NO: 6
MSPCGRARRQTSRGAMAVLAWKPPRTRLPMGASALCVVVL
CVVLYIFPVYRLPNEKEIVQGVLQQGTAWRRNQTAARAFR
KQMEDCCDPAHLFAMTKMNSPMGKSMWYDGEFLYSFTIDN
STYSLFPQATPFQLPLKKCAVVGNGGILKKSGCGRQIDEA
NFVMRCNLPPLSSEYTKDVGSKSQLVTANPSIIRQRFQNL
LWSRKTFVDNMKTYNHSYIYMPAFSMKTGTEPSLRVYYTL
SDVGANQTVLFANPNFLRSIGKFWKSRGIHAKRLSTGLFL
VSAALGLCEEVAIYGFWPFSVNMHEQPISHHYYDNVLPFS
GFHAMPEEFLQLWYLHKIGALRMQLDPCEDTSLQPTS
SEQ ID NO: 7
GSGATNFSLLKQAGDVEENPGP
SEQ ID NO: 8
MEEGFRDRAAFIRGAKDIAKEVKKHAAKKVVKGLDRVQDE
YSRRSYSRFEEEDDDDDFPAPSDGYYRGEGTQDEEEGGAS
SDATEGHDEDDEIYEGEYQGIPRAESGGKGERMADGAPLA
GVRGGLSDGEGPPGGRGEAQRRKEREELAQQYEAILRECG
HGRFQWTLYFVLGLALMADGVEVFVVGFVLPSAEKDMCLS
DSNKGMLGLIVYLGMMVGAFLWGGLADRLGRRQCLLISLS
VNSVFAFFSSFVQGYGTFLFCRLLSGVGIGGSIPIVFSYF
SEFLAQEKRGEHLSWLCMFWMIGGVYAAAMAWAIIPHYGW
SFQMGSAYQFHSWRVFVLVCAFPSVFAIGALTTQPESPRF
FLENGKHDEAWMVLKQVHDTNMRAKGHPERVFSVTHIKTI
HQEDELIEIQSDTGTWYQRWGVRALSLGGQVWGNFLSCFG
PEYRRITLMMMGVWFTMSFSYYGLTVWFPDMIRHLQAVDY
ASRTKVFPGERVEHVTFNFTLENQIHRGGQYFNDKFIGLR
LKSVSFEDSLFEECYFEDVTSSNTFFRNCTFINTVFYNTD
LFEYKFVNSRLINSTFLHNKEGCPLDVTGTGEGAYMVYFV
SFLGTLAVLPGNIVSALLMDKIGRLRMLAGSSVMSCVSCF
FLSFGNSESAMIALLCLFGGVSIASWNALDVLTVELYPSD
KRTTAFGFLNALCKLAAVLGISIFTSFVGITKAAPILFAS
AALALGSSLALKLPETRGQ VLQ
SEQ ID NO: 9
MEDSYKDRTSLMKGAKDIAREVKKQTVKKVNQAVDRAQDE
YTQRSYSRFQDEEDDDDYYPAGETYNGEANDDEGSSEATE
GHDEDDEIYEGEYQGIPSMNQAKDSIVSVGQPKGDEYKDR
RELESERRADEEELAQQYELIIQECGHGRFQWALFFVLGM
ALMADGVEVFVVGFVLPSAETDLCIPNSGSGWLGSIVYLG
MMVGAFFWGGLADKVGRKQSLLICMSVNGFFAFLSSFVQG
YGFFLFCRLLSGFGIGGAIPTVFSYFAEVLAREKRGEHLS
WLCMFWMIGGIYASAMAWAIIPHYGWSFSMGSAYQFHSWR
VFVIVCALPCVSSWALTFMPESPRFLLEVGKHDEAWMILK
LIHDTNMRARGQPEKVFTVNKIKTPKQIDELIEIESDTGT
WYRRCFVRIRTELYGIWLTFMRCFNYPVRDNTIKLTIWFT
LSFGYYGLSVWFPDVIKPLQSDEYALLTRNVERDKYANFT
INFTMENQIHTGMEYDNGRFIGVKFKSVTFKDSVFKSCTF
EDVTSVNTYFKNCTFIDTVFDNTDFEPYKFIDSEFKNCS
FFHNKTGCQITFDDDYSAYWIYFVNFLGTLAVLPGNIVSA
LLMDRIGRLTMLGGSMVLSGISCFFLWFGTSESMMIGMLC
LYNGLTISAWNSLDWTVELYPTDRRATGFGFLNALCKAA
AVLGNLIFGSLVSITKSIPILLASTVLVCGGLVGLCLPD
TRTQVLM
SEQ ID NO: 10
MDDYKYQDNYGGYAPSDGYYRGNESNPEEDAQSDVTEGHD
EEDEIYEGEYQGIPHPDDVKAKQAKMAPSRMDSLRGQTDL
MAERLEDEEQLAHQYETIMDECGHGRFQWILFFVLGLALM
ADGVEVFVVSFALPSAEKDMCLSSSKKGMLGMIVYLGMMA
GAFILGGLADKIGRKRVLSMSLAVNASFASLSSFYQGYGA
FLFCRLISGIGIGGALPIVFAYFSEFLSREKRGEHLSWLG
IFWMTGGLYASAMAWSIIPHYGWGFSMGTNYHFHSWRVFV
IVCALPCTVSMVALKFMPESPRFLLEMGKHDEAWMILKQV
HDTNMRAKGTPEKVFTVSNIKTPKQMDEFIEIQSSTGTWY
QRWLVRFKTIFKQVWDNALYCVMGPYRMNTLILAVVWFAM
AFSYYGLTVWFPDMIRYFQDEEYKSKMKVFFGEHVYGATI
NFTMENQIHQHGKLVNDKFTRMYFKHVLFEDTFFDECYFE
DVTSTDTYFKNCTIESTIFYNTDLYEHKFINCRFINSTFL
EQKEGCHMDLEQDNDFLIYLVSFLGSLSVLPGNIISALLM
DRIGRLKMIGGSMLISAVCCFFLFFGNSESAMIGWQCLFC
GTSIAAWNALDVITVELYPTNQRATAFGILNGLCKFGAIL
GNTIFASFVGITKVVPILLAAASLVGGGLIALRLPETREQ
VLM
SEQ ID NO: 11
FPDMIRHLQAVDYASRTKVFPGERVEHVTFNFTLENQIHR
GGQYFNDKFIGLRLKSVSFEDSLFEECYFEDVTSSNTFF
RNCTFINTVFYNTDLFEYKFVNSRLINSTFLHNKEGCPLD
VTGTGEGAY
SEQ ID NO: 12
WFPDMIRYFQDEEYKSKMKVFFGEHVYGATINFTMENQIH
QHGKLVNDKFTRMYFKHVLFEDTFFDECYFEDVTSTDTYF
KNCTIESTIFYNTDLYEHKFINCRFINSTFLEQKEGCHMD
LEQDNDFLIY
SEQ ID NO: 13
WFPDVIKPLQSDEYALLTRNVERDKYANFTINFTMENQIH
TGMEYDNGRFIGVKFKSVTFKDSVFKSCTFEDVTSVNTYF
KNCTFIDTVFDNTDFEPYKFIDSEFKNCSFFHNKTGCQI
TFDDDYSAY
SEQ ID NO: 14
MVSESHHEALAAPPVTTVATVLPSNATEPASPGEGKEDAF
SKLKEKFMNELHKIPLPPWALIAIAIVAVLLVLTCCFCIC
KKCLFKKKNKKKGKEKGGKNAINMKDVKDLGKTMKDQALK
DDDAETGLTDGEEKEEPKEEEKLGKLQYSLDYDFQNNQLL
VGIIQAAELPALDMGGTSDPYVKVFLLPDKKKKFETKVHR
KTLNPVFNEQFTFKVPYSELGGKTLVMAVYDFDRFSKHDI
IGEFKVPMNTVDFGHVTEEWRDLQSAEKEEQEKLGDICFS
LRYVPTAGKLTVVILEAKNLKKMDVGGLSDPYVKIHLMQN
GKRLKKKKTTIKKNTLNPYYNESFSFEVPFEQIQKVQVVV
TVLDYDKIGKNDAIGKVFVGYNSTGAELRHWSDMLANPRR
PIAQWHTLQVEEEVD AMLAVKK
SEQ ID NO: 15
MRNIFKRNQEPIVAPATTTATMPIGPVDNSTESGGAGESQ
EDMFAKLKEKLFNEINKIPLPPWALIAIAWAGLLLLTCCF
CICKKCCCKKKKNKKEKGKGMKNAMNMKDMKGGQDDDDAE
TGLTEGEGEGEEEKEPENLGKLQFSLDYDFQANQLTVGVL
QAAELPALDMGGTSDPYVKVFLLPDKKKKYETKVHRKTLN
PAFNETFTFKVPYQELGGKTLVMAIYDFDRFSKHDIIGEV
KVPMNTVDLGQPIEEWRDLQGGEKEEPEKLGDICTSLRYV
PTAGKLTVCILEAKNLKKMDVGGLSDPYVKIHLMQNGKRL
KKKKTTVKKKTLNPYFNESFSFEIPFEQIQKVQVVVTVLD
YDKLGKNEAIGKIFVGSNATGTELRHWSDMLANPRRPIAQ
WHSLKPEEEVDA LLGKNK
SEQ ID NO: 16
MVSKGEAVIKEFMRFKVHMEGSMNGHEFEIEGEGEGRPYE
GTQTAKLKVTKGGPLPFSWDILSPQFMYGSRAFTKHPADI
PDYYKQSFPEGFKWERVMNFEDGGAVTVTQDTSLEDGTLI
YKVKLRGTNFPPDGPVMQKKTMGWEASTERLYPEDGVLKG
DIKMALRLKDGGRYLADFKTTYKAKKPVQMPGAYNVDRKL
DITSFINEDYTVVEQYERSEGRHSTGGMDELYK
SEQ ID NO: 17
MASLPATHELHIFGSINGVDFDMVGQGTGNPNDGYEELNL
KSTKGDLQFSPWILVPHIGYGFHQYLPYPDGMSPFQAAMV
DGSGYQVHRTMQFEDGASLTVNYRYTYEGSHIKGEAQVKG
TGFPADGPVMTNSLTAADWCRSKKTYPNDKTIISTFKWSY
TTGNGKRYRSTARTTYTFAKPMAANYLKNQPMYVFRKTEL
KHSKTELNFKEWQKAFTGFEDFVGDWRQTAGYNLDQVLEQG
GVSSLFQ
SEQ ID NO: 18
LFTGVVPILVELDGDVNGHKFSVSGEGEGDATYGKLTLKF
ICTTGKLPVPWPTLVTTLSWGVQCFARYPDHMKQHDFFKS
AMPEGYVQERTIFFKDDGNYKTRAEVKFEGDTLVNRIELK
GIDFKEDGNILGHKLEYNYFSDNVYITADKQKNGIKANFK
IRHNIEDGGVQLADHYQQNTPIGDGPVLLPDNHYLSTQSK
LSKDPNEKRDHMVLLEFVTAAGL
SEQ ID NO: 19
MAEDADMRNELEEMQRRADQLADESLESTRRMLQLVEESK
DAGIRTLVMLDEQGEQLERIEEGMDQINKDMKEAEKNLTDL
GKFCGLCVCPCNKLKSSDAYKKAWGNNQDGVVASQPARVVD
EREQMAISGGFIRRVTNDARENEMDENLEQVSGIIGNLRHM
ALDMGNEIDTQNRQIDRIMEKADSNKTRIDEANQRATKMLG
SG
SEQ ID NO: 20
MIEQDGLHAGSPAAWVERLFGYDWAQQTIGCSDAAVFRLS
AQGRPVLFVKTDLSGALNELQDEAARLSWLATTGVPCAAV
LDVVTEAGRDWLLLGEVPGQDLLSSHLAPAEKVSIMADA
MRRLHTLDPATCPFDHQAKHRIERARTRMEAGLVDQD
DLDEEHQGLAPAELFARLKARMPDGEDLVVTHGDACLPNI
MVENGRFSGFIDCGRLGVADRYQDIALATRDIAEELGGEW
ADRFLVLYGLAAPDSQRIAFYRLLD EFF
SEQ ID NO: 21
MTEYKPTVRLATRDDVPRAVRTLAAAFADYPATRHTVDPD
RHIERVTELQELFLTRVGLDIGKVWVADDGAAVAVWTTPE
SVEAGAVFAEIGPRMAELSGSRLAAQQQMEGLLAPHRPKE
PAWFLATVGVSPDHQGKGLGSAVVLPGVEAAERAGVPAFLE
TSAPRNLPFYERLGFTVTADVEVPEGPRTWCMTRKPGA
SEQ ID NO: 22
HTLEDFVGDWRQTAGYNLDQVLEQGGVSSLFQNLGVSVTP
IQRIVLSGENGLKIDIHVIIPYEGLSGDQMGQIEKIFKVV
YPVDDHHFKVILHYGTLVIDGVTPNMIDYFGRPYEGIAVF
DGKKITVTGTLWNGNKIIDERLINPDGSLLFRVTINGVTG
WRLCERILA

1. A cell that has been genetically engineered to express or overexpress a clostridial neurotoxin receptor, or a variant or fragment thereof.

2. An in vitro method for characterizing the activity of a clostridial neurotoxin formulation or identifying a clostridial neurotoxin formulation for therapeutic (and/or cosmetic) use, said method comprising:
a. providing a cell having an exogenous nucleic acid that provides for expression or overexpression of a receptor and/or ganglioside having binding affinity for the clostridial neurotoxin, and an exogenous nucleic acid that provides for expression or overexpression of an indicator protein cleavable by the clostridial neurotoxin;
b. contacting said cell with the clostridial neurotoxin formulation;
c. comparing a level of cleavage of the indicator protein subsequent to contact with the clostridial neurotoxin formulation with a level of cleavage pre-contact with the clostridial neurotoxin formulation; and
d. identifying (i) the clostridial neurotoxin formulation as being suitable for therapeutic (and/or cosmetic) use when the level of cleavage of the indicator protein subsequent to the contact is increased, or identifying (ii) the presence of activity when the level of cleavage of the indicator protein subsequent to the contact is increased; or
e. identifying (i) the clostridial neurotoxin formulation as being unsuitable for therapeutic (and/or cosmetic) use when the level of cleavage of the indicator protein subsequent to the contact is not increased, or identifying (ii) the absence of activity when the level of cleavage of the indicator protein subsequent to the contact is not increased.

3. An in vitro method for characterizing the activity of a clostridial neurotoxin formulation or identifying a clostridial neurotoxin formulation that is suitable for therapeutic (and/or cosmetic) use, said method comprising:
a. providing a cell having an exogenous nucleic acid that provides for expression or overexpression of a receptor and/or ganglioside having binding affinity for the clostridial neurotoxin, and an exogenous nucleic acid that provides for expression or overexpression of an indicator protein cleavable by the clostridial neurotoxin;
b. contacting said cell with the clostridial neurotoxin formulation;
c. comparing a level of cleavage of the indicator protein subsequent to contact with the clostridial neurotoxin formulation with a level of cleavage subsequent to contact with a control clostridial neurotoxin formulation; and
d. identifying (i) the clostridial neurotoxin formulation as being suitable for therapeutic (and/or cosmetic) use when the level of cleavage of the indicator protein subsequent to the contact is increased or equivalent to a level of cleavage subsequent to contact with a control clostridial neurotoxin formulation, or identifying (ii) the presence of activity when the level of cleavage of the indicator protein subsequent to the contact is increased or equivalent to a level of cleavage subsequent to contact with a control clostridial neurotoxin formulation; or
e. identifying (i) the clostridial neurotoxin as being unsuitable for therapeutic (and/or cosmetic) use when the level of cleavage of the indicator protein subsequent to the contact is not increased or equivalent to a level of cleavage subsequent to contact with a control clostridial neurotoxin formulation, or identifying (ii) the absence of activity when the level of cleavage of the indicator protein subsequent to the contact is not increased or not equivalent to a level of cleavage subsequent to contact with a control clostridial neurotoxin formulation.

4. The cell or method according to any one of the preceding claims, wherein a cell that overexpresses the receptor and/or ganglioside expresses more of the receptor and/or ganglioside when compared with a natural target of the clostridial neurotoxin.

5. The cell or method according to any one of the preceding claims, wherein a cell that overexpresses the receptor and/or ganglioside expresses more of the receptor and/or ganglioside when compared with a cell lacking the exogenous nucleic acid.

6. The cell or method of any one of the preceding claims, wherein the cell is a neuronal cell, a non-neuronal cell, a neuroendocrine cell, an embryonic kidney cell, a breast cancer cell, a neuroblastoma cells, or a neuroblastoma-glioma hybrid cell; preferably a non-neuronal cell.

7. The cell or method of any one the preceding claims, wherein the cell is a neuroblastoma cell or a neuroblastoma-glioma cell.

8. The cell or method of any one of the preceding claims, wherein the cell is a neuroblastoma-glioma cell.

9. The cell or method of any one of the preceding claims, wherein the cell is an NG108 cell.

10. The cell or method of any one of the preceding claims, wherein the cell has been genetically engineered to express or overexpress a ganglioside.

11. The cell or method of any one of the preceding claims, wherein the cell has been genetically engineered to express or overexpress GM1a, GD1a, GD1b, GT1b, and/or GQ1b.

12. The cell or method of any one of the preceding claims, wherein the cell has been genetically engineered to express or overexpress GD1a, GD1b, and/or GT1b.

13. The cell or method of any one of the preceding claims, wherein the cell has been genetically engineered to express or overexpress GD1b and/or GT1b.

14. The cell or method of any one of the preceding claims, wherein the cell has been genetically engineered to express or overexpresses an enzyme of the ganglioside synthesis pathway, or a variant or fragment thereof that has the catalytic activity of such an enzyme.

15. The cell or method of any one of the preceding claims, wherein the cell has been genetically engineered to express or overexpress glucosylceramide synthase, GalT-I, GalNAcT, GM3 synthase, GD3 synthase, GT3 synthase, galactosylceramide synthase, GM4 synthase, GalT-II, ST-IV, or ST-V, or a variant or fragment thereof that has the catalytic activity of such an enzyme.

16. The cell or method of any one of the preceding claims, wherein the cell has been genetically engineered to express or overexpress GD3 synthase, or a variant or fragment thereof that has the catalytic activity of GD3 synthase.

17. The cell or method of any one of the preceding claims, wherein the cell has been genetically engineered to express or overexpress GD3 synthase.

18. The cell or method of any one of the preceding claims, wherein the cell has been genetically engineered to express or overexpress a protein receptor, or a variant or fragment thereof that has the ability to bind clostridial neurotoxin.

19. The cell or method of any one of the preceding claims, wherein the cell has been genetically engineered to express or overexpress a SV2 or a synaptotagmin, or a variant or fragment thereof that has the ability to bind clostridial neurotoxin.

20. The cell or method of any one of the preceding claims, wherein the cell has been genetically engineered to express or overexpress a SV2, or a variant or fragment thereof that has the ability to bind clostridial neurotoxin.

21. The cell or method of any one of the preceding claims, wherein the cell has been genetically engineered to express or overexpress SV2A or SV2C (preferably SV2A), or a variant or fragment thereof that has the ability to bind clostridial neurotoxin.

22. The cell or method of any one of the preceding claims, wherein the cell has been genetically engineered to express or overexpress the fourth luminal domain of SV2A or SV2C.

23. The cell or method of any one of the preceding claims, wherein the cell has been genetically engineered to express or overexpress an indicator protein.

24. The cell or method of any one of the preceding claims, wherein the cell has been genetically engineered to express or overexpress an indicator protein comprising a SNARE domain.

25. The cell or method of any one of the preceding claims, wherein the cell has been genetically engineered to express or overexpress an indicator protein, the indicator protein comprising the amino acid sequence of syntaxin, synaptobrevin, or SNAP-25, or a variant or fragment thereof that is susceptible to proteolysis by the protease component of a wild-type clostridial neurotoxin.

26. The cell or method of any one of the preceding claims, wherein the cell has been genetically engineered to express or overexpress a labelled indicator protein.

27. The cell or method of any one of the preceding claims, wherein the cell has been genetically engineered to express or overexpress an indicator protein comprising an N-terminal label and a C-terminal label.

28. The cell or method of any one of the preceding claims, wherein the cell has been genetically engineered to express or overexpress an indicator protein comprising the amino acid sequence of a fluorescent protein label.

29. The cell or method of any one of the preceding claims, wherein the cell has been genetically engineered to express or overexpress an indicator protein comprising the amino acid sequence of mScarlet and the amino acid sequence of NeonGreen.

30. The cell or method of any one of the preceding claims, wherein the cell has been genetically engineered to express or overexpress an indicator protein comprising mScarlet as an N-terminal label and NeonGreen as a C-terminal label.

31. A method of producing the cell of any one of claim 1 or 4-30, wherein the method comprises introducing into a cell a nucleic acid encoding: a clostridial neurotoxin receptor, or a variant or fragment thereof that has the ability to bind clostridial neurotoxin; and/or an enzyme of the ganglioside synthesis pathway, or a variant or fragment thereof that has the catalytic activity of such enzyme.

32. The method of claim 31, wherein the method further comprises introducing into the cell a nucleic acid encoding an indicator protein.

33. The method of claims 31-32, wherein the nucleic acid is introduced by transfection.

34. An assay for determining the activity of a modified or recombinant neurotoxin, the method comprising contacting the cell any one of claim 1 or 4-30 with the modified or recombinant neurotoxin under conditions and for a period of time that would allow the protease domain of a wild-type clostridial neurotoxin to cleave an indicator protein in the cell and determining the presence of product resulting from the cleavage of such an indicator protein.

35. The assay of claim 30, wherein the full-length indicator protein is not readily degraded in the cell but, following cleavage thereof, one of the resulting fragments is.

36. The assay of any one of claims 30-31, wherein the indicator protein is labeled.

37. The assay of any one of claims 30-32, wherein the indicator protein comprises a C-terminal label and full-length indicator protein is not readily degraded in the cell but, following cleavage thereof, the resulting C-terminal fragment is and the degradation of the C-terminal fragment results in the degradation of the C-terminal label.

38. The assay of any one of claims 30-33, wherein the indicator protein comprises a C-terminal label and the full-length indicator protein is not readily degraded in the cell but, following cleavage thereof, the resulting C-terminal fragment is and the degradation of the C-terminal fragment results in the degradation of the C-terminal label and cleavage of the indicator protein is determined by measuring the signal from the C-terminal label following the contacting of the cell with the modified or recombinant neurotoxin.

39. The assay of claim 30 wherein, following such contact, the cell is lysed and the resulting cell lysate is contacted with antibodies and a Western blot performed.

40. A method for engineering a cell suitable for use in an assay for characterizing the activity of a clostridial neurotoxin formulation or identifying a clostridial neurotoxin formulation that is suitable for therapeutic (and/or cosmetic) use, the method comprising:
a. manipulating a cell to incorporate:
i. an exogenous nucleic acid that provides for expression or overexpression of a receptor and/or ganglioside having binding affinity for the clostridial neurotoxin; and
ii. an exogenous nucleic acid that provides for expression or overexpression of an indicator protein cleavable by the clostridial neurotoxin.


SEQUENCE LISTING (22 sequences)

SEQ ID NO: 1 - DNA, 2502 bp, Artificial Sequence
  Synthetic
atggtgtcgaagggggaagcggtgatcaaggagttcatgaggtttaaagtgcatatggagggatctatgaacggacacgagtttgagatcgaaggggaaggagaggggcgcccatacgaaggcacccagactgccaagctgaaagtcacaaagggtggacccttgcccttctcgtgggatattctgagcccgcaattcatgtacgggtcccgggccttcaccaagcaccctgctgacattccggattactataagcagagcttcccggaaggcttcaaatgggagcgagtgatgaacttcgaggatggaggcgccgtgaccgtgactcaggacacttcactggaagatggcactctgatctacaaggtcaagctgcggggcaccaacttcccaccggacggaccggtcatgcagaaaaagaccatgggatgggaggcctccaccgagcgcctgtaccccgaagatggagtcctcaagggggacatcaagatggccctgcggctcaaggatggtggaagatacctggctgacttcaagaccacgtacaaggccaagaagccagtccagatgcccggcgcgtacaatgtggatcgcaagctggacatcacttcccacaacgaggactacaccgtggtggagcagtacgaacggtccgagggtcggcactccactggtggcatggacgagctgtacaaaatggccgaggatgcagacatgagaaacgaactggaagaaatgcagcggagagcagaccagctcgcggacgaatcactggaatcgacccgccggatgcttcaactggtcgaggaatcaaaggacgcgggtatccggacccttgtgatgctggacgaacagggagagcagctggagaggatcgaagagggaatggaccagattaacaaggacatgaaggaagcggaaaagaacctcaccgaccttggaaagttctgcgggttgtgcgtgtgtccgtgcaacaagctgaagtcctccgacgcctacaagaaggcctggggaaacaaccaggacggtgtcgtggcttcccaacccgcacgggtggtggatgagcgggaacagatggcgatttccggaggcttcattagacgcgtgaccaacgacgcccgcgaaaacgagatggacgaaaacctggaacaagtgtcgggaatcatcggaaacttgagacacatggccctcgacatgggcaacgaaattgatacacagaaccggcagattgaccggatcatggaaaaggcagactcaaacaagactcggattgacgaagcgaaccagagggccactaagatgttgggttccgggatggtgtcaaagggagaagaagacaacatggcatcactgcccgccacccacgagctgcacatcttcggttccatcaacggggtcgacttcgacatggtcggccagggaactggaaacccgaatgacggttatgaagaactgaaccttaaatcaaccaagggggaccttcagttctcgccctggattttggtccctcacattggatacggattccatcagtatctgccgtaccccgacggaatgagcccgttccaggctgccatggtggacggatcgggataccaggtccaccgcaccatgcagtttgaagatggcgcaagcctgaccgtgaactaccggtatacctacgagggctcacacatcaagggggaagcccaagtcaagggtaccggcttcccggccgacggaccagtgatgaccaactccttgaccgccgccgactggtgccgcagcaagaaaacttaccccaacgataagacaatcatctccactttcaagtggtcctacaccacgggcaacggcaaacgctaccgaagcactgcacggaccacctacactttcgcgaagcctatggccgccaactacctgaagaaccagccgatgtacgtgttcagaaagacggaactcaagcactccaaaaccgaactgaactttaaggagtggcagaaggctttcactggattcgaggactttgtcggcgactggcgccagactgccggctacaacctggaccaagtgctcgaacaggggggtgtctccagcctcttccaaaatctgggcgtgtccgtgaccccgatccagcggatcgtgctcagcggggaaaacggcctgaagatcgatatccacgtcatcatcccgtacgagggactgagcggcgaccagatgggtcagatcgaaaagattttcaaggtggtctatcccgtggatgaccaccacttcaaagtgatcctgcattacgggaccctcgtgatcgacggcgtcaccccgaacatgattgattacttcggacggccttatgaagggatcgccgtgttcgacggcaaaaagatcaccgtgactggcaccctgtggaacggaaataagatcattgacgagcggctgatcaacccagacgggtcgctgctgttccgcgtgaccatcaacggagtgaccggctggcggctgtgcgagcgcatcctcgcctgatag

SEQ ID NO: 2 - DNA, 2517 bp, Artificial Sequence
  Synthetic
atggtgtcgaagggggaagcggtgatcaaggagttcatgaggtttaaagtgcatatggagggatctatgaacggacacgagtttgagatcgaaggggaaggagaggggcgcccatacgaaggcacccagactgccaagctgaaagtcacaaagggtggacccttgcccttctcgtgggatattctgagcccgcaattcatgtacgggtcccgggccttcaccaagcaccctgctgacattccggattactataagcagagcttcccggaaggcttcaaatgggagcgagtgatgaacttcgaggatggaggcgccgtgaccgtgactcaggacacttcactggaagatggcactctgatctacaaggtcaagctgcggggcaccaacttcccaccggacggaccggtcatgcagaaaaagaccatgggatgggaggcctccaccgagcgcctgtaccccgaagatggagtcctcaagggggacatcaagatggccctgcggctcaaggatggtggaagatacctggctgacttcaagaccacgtacaaggccaagaagccagtccagatgcccggcgcgtacaatgtggatcgcaagctggacatcacttcccacaacgaggactacaccgtggtggagcagtacgaacggtccgagggtcggcactccactggtggcatggacgagctgtacaaaatggccgaggatgcagacatgagaaacgaactggaagaaatgcagcggagagcagaccagctcgcggacgaatcactggaatcgacccgccggatgcttcaactggtcgaggaatcaaaggacgcgggtatccggacccttgtgatgctggacgaacagggagagcagctggagaggatcgaagagggaatggaccagattaacaaggacatgaaggaagcggaaaagaacctcaccgaccttggaaagttctgcgggttgtgcgtgtgtccgtgcaacaagctgaagtcctccgacgcctacaagaaggcctggggaaacaaccaggacggtgtcgtggcttcccaacccgcacgggtggtggatgagcgggaacagatggcgatttccggaggcttcattagacgcgtgaccaacgacgcccgcgaaaacgagatggacgaaaacctggaacaagtgtcgggaatcatcggaaacttgagacacatggccctcgacatgggcaacgaaattgatacacagaaccggcagattgaccggatcatggaaaaggcagactcaaacaagactcggattgacgaagcgaaccagagggccactaagatgttgggttccgggatggtgtcaaagggagaagaactgttcactggagtggtgcccatcctggtggagctggatggcgatgtgaacggccataaattctcagtcagcggagagggagagggcgatgcgacttacggaaagctgactttgaagtttatctgcactaccggaaagctgcctgtgccatggcctaccctcgtgaccaccctgtcctggggcgtccaatgtttcgcacgctaccctgaccatatgaagcagcacgacttcttcaagtccgccatgcccgagggctacgtgcaggaacgcaccatcttcttcaaggacgacgggaactacaaaaccagggctgaagtgaagttcgagggagacaccctggtcaatcggattgaattgaagggaatcgatttcaaggaagatggaaacatcctgggacataagcttgagtacaactacttctccgacaacgtgtacatcacggccgataagcagaagaacggaatcaaagctaacttcaagattcggcacaacattgaggacggcggcgtccagctggcggaccattatcagcagaatacccctattggggatggaccggtgctgctcccggacaaccattacctgtccacccaatctaagctgagcaaggacccaaacgagaagcgcgatcacatggtgctgctcgagttcgtgactgccgccgggcttcacacacttgaggactttgtcggcgactggcgccagactgccggctacaacctggaccaagtgctcgaacaggggggtgtctccagcctcttccaaaatctgggcgtgtccgtgaccccgatccagcggatcgtgctcagcggggaaaacggcctgaagatcgatatccacgtcatcatcccgtacgagggactgagcggcgaccagatgggtcagatcgaaaagattttcaaggtggtctatcccgtggatgaccaccacttcaaagtgatcctgcattacgggaccctcgtgatcgacggcgtcaccccgaacatgattgattacttcggacggccttatgaagggatcgccgtgttcgacggcaaaaagatcaccgtgactggcaccctgtggaacggaaataagatcattgacgagcggctgatcaacccagacgggtcgctgctgttccgcgtgaccatcaacggagtgaccggctggcggctgtgcgagcgcatcctcgcctgatag

SEQ ID NO: 3 - DNA, 2250 bp, Artificial Sequence
  Synthetic
atgaccatggatgagcagcaatcgcaggctgtagccccggtatatgtcggtggtatggatgagaaaacgactgggtggcggggtggacacgtcgtcgagggcctggcaggcgaacttgaacaactgcgggctcgcttggagcaccacccgcaaggacagcgcgagccgtccatggtgtcaaagggggaggaactgtttactggggtcgtccctatcttggtggaactcgacggggatgtgaacggacacaagttttcggtatccggggaaggcgagggggatgccacgtatggaaagctcacacttaagttcatctgcacgacagggaagctcccagtgccttggcccacgttggtgactacgctcacatggggtgtccagtgcttcgcacggtatcccgaccacatgaagcagcatgatttctttaagtcagccatgccggagggatatgtacaagaaaggaccatcttcttcaaagatgacggtaactacaagaccagagccgaggtaaagtttgaaggcgacactctcgtgaacaggattgagctgaagggaattgatttcaaagaggatgggaacatccttggtcacaaattggagtacaatgccatttcggataacgtgtacattacagcggataagcagaagaatgggatcaaagcgaatttcaaaatcaggcataacatcgaggacgggtcggtgcagctcgccgaccattaccagcagaatacgcccatcggagatggacccgtacttctgcccgacaatcattatctgtcaacgcaatcagcgcttagcaaagatcccaatgagaaaagggaccacatggtgctcctcgaatttgtgacggcagcgggaattaccctcgggatggacgaactgtacaaaagcgggttgagactcgagcgctgaactcgagatgccttttgtcaacaagcagtttaactataaggatcccgtgaatggtgtggacattgcctacatcaagattccaaacgctggacaaatgcagcccgtcaaggctttcaaaattcacaacaagatctgggtgatcccggagagggacacctttaccaatccagaagagggcgaccttaaccctccgccagaggccaaacaggtgcccgtgagctattacgactcaacttatctctccaccgacaacgaaaaggacaattacctcaagggagtcaccaagctgttcgaacggatctactctaccgatctcggcaggatgctcctgacttctatcgtgcggggcatccccttctggggtgggagcaccattgacaccgaactgaaggtgattgataccaattgcatcaacgtcatccagccagacggttcctaccggtctgaggagctcaatcttgtgattattggcccgtcagctgatatcatccagttcgaatgcaagtctttcggacacgacgtgcttaatctcacccgcaatggttacggaagcacccagtacatcagattctctccggactttactttcggatttgaagagtcactggaagtcgacaccaatcctctgctcggagccggaaagttcgccaccgaccctgcagtgacccttgctcacgagctgattcatgcagagcatcgcctgtacgggatcgccatcaatcctaaccgcgtgtttaaggtcaataccaacgcttactatgaaatgagcggactggaggtgtccttcgaggaactgcgcaccttcggaggtcatgacgctaagttcatcgactcactgcaagagaatgagttccggctgtactattacaacaagtttaaggatgtcgcctcaactctgaacaaggccaaaagcatcatcggcaccaccgccagcctgcaatacatgaaaaacgtgttcaaggaaaagtaccttcttagcgaagatacttccgggaagttttcagtcgacaaactgaagttcgacaagctgtacaagatgctcaccgaaatctacaccgaggacaattttgtgaacttcttcaaagtgattaacagaaagacctatctgaacttcgacaaagccgtgttccggattaacattgtgcccgatgagaactacactatcaaggacgggttcaaccttaagggtgcaaatctttcaactaatttcaacggacagaatactgagatcaattcaaggaacttcactagactcaagaatttcactgggcttttcgagttctataagctgctgtgtgtccgcggaattatccccttcaagtgaagcttcgtcaatga

SEQ ID NO: 4 - DNA, 5772 bp, Artificial Sequence
  Synthetic
atgtccccatgtggacgagcgcgcagacagacctcaagaggagcgatggccgtgctggcctggaagttcccgaggactcgcctgcccatgggagcctctgctctgtgtgtggtcgtgctgtgttggctgtacatcttcccggtgtaccggctgcctaacgaaaaggaaattgtgcagggcgtgctccagcaggggaccgcttggcggcgcaaccagaccgctgcgagggcttttcggaagcagatggaagattgttgcgaccccgcccatcttttcgcgatgaccaagatgaacagcccgatgggaaagtccatgtggtacgacggagagttcctgtattccttcaccattgacaacagcacttactcactgttcccgcaagccacccccttccaactgccgcttaagaagtgcgccgtcgtggggaacggcggcatcctcaagaagtccggatgcgggcgccagattgatgaagccaacttcgtgatgcggtgcaatctcccgccactctcgtccgagtacaccaaggacgtggggtcaaagtcgcagctcgtcaccgccaacccttcgatcatcagacaacggttccagaaccttctgtggagccggaaaacatttgtggataacatgaagatctacaaccattcctacatctatatgcctgccttctccatgaaaactggaaccgaaccctccctgagagtgtactacaccctgtccgacgtgggcgcaaaccagaccgtccttttcgccaaccccaacttcctgcgctccatcggaaagttctggaagtccagaggcattcacgcgaaacgcttgtccactggattgttcttggtgtccgccgctctgggcctgtgcgaggaagtggccatatacggattctggcctttctccgtcaacatgcacgagcagcccatctcccaccattattacgacaatgtcctgcctttctcgggatttcacgcgatgcccgaggagttcttgcaactgtggtaccttcacaagatcggtgccctgcggatgcagctggacccttgcgaggacacctcgctgcaacccacctcggagcagaaactcatttccgaagaggatctgaacggggagcagaagctcatctccgaggaggacctgaacggagaacagaagctgattagcgaagaggacctgggcagcggtgccaccaatttttctctgctcaagcaggccggagatgtggaagagaacccgggtcccatggaggactcctacaaagatcggacttctctgatgaagggagccaaggacatcgccagggaagtgaagaagcaaaccgtcaagaaggtcaaccaggccgtggacagagcccaggacgagtacacccagcggtcgtactcgcggttccaggatgaagaggatgacgacgactactaccctgccggcgaaacctataatggggaagccaacgatgacgaaggctccagcgaagccactgaaggacacgacgaggacgacgaaatctacgaaggagaataccagggcatcccttcgatgaatcaggccaaagattcaattgtgtcagtgggacagcctaagggcgacgagtacaaggaccggagagagctcgaaagcgagcggagggccgacgaagaggaactggcacaacagtacgagctgatcatccaggagtgtgggcacggccggttccagtgggcgctgttcttcgtgctcggaatggcactgatggccgacggcgtggaagtgttcgtggtcggattcgtgctgccctcggccgaaaccgacctctgcattcccaactccggctcgggatggctggggtccatcgtgtacctgggaatgatggtcggcgccttcttctggggtggcctggcagacaaggtcggccggaagcagtccctcttgatctgcatgagcgtcaacggatttttcgccttcctgtcatcattcgtgcaaggttacgggttcttccttttctgccgcctgctgtccggctttgggatcggcggggctattccgactgtgttctcctactttgccgaagtgctggctcgcgaaaaacggggcgaacacctttcctggctgtgtatgttctggatgatcggcggcatctacgcctcggccatggcctgggctattatcccgcattatgggtggtccttctcaatgggaagcgcataccagttccattcgtggcgggtgttcgtgatcgtgtgcgccctcccgtgtgtgtcctccgtggtggctctgacattcatgccggagtcacctcggttcttgttggaagtcgggaagcacgacgaagcctggatgattctgaagctgatccacgacactaatatgcgggcccggggacagcctgagaaagtgttcaccgtcaacaagattaagaccccgaagcaaatcgatgaactgattgaaattgagtccgacaccggaacttggtaccgccggtgcttcgtgcggattcgcaccgagctgtacggaatctggctcaccttcatgcgctgcttcaactaccccgtgcgcgacaacaccatcaagctgaccatcgtgtggttcactctgtctttcggctactatgggctgtcagtgtggttcccggatgtcatcaagccgctccaatccgatgaatacgccctgctgacccgcaatgtggagagagacaaatacgccaacttcaccatcaatttcaccatggaaaaccagattcacaccggaatggagtacgacaatggacgattcatcggagtgaagttcaagagcgtgaccttcaaggactcggtgttcaagtcctgtaccttcgaggacgtgaccagcgtgaacacttattttaagaattgcaccttcatcgatactgtgttcgataacaccgacttcgagccctataagttcattgactcggagttcaagaactgttcattcttccacaacaagactggttgccagatcaccttcgatgacgactacagcgcctactggatctactttgtgaactttttgggaactctcgcagtgcttcctggcaacattgtgtccgcactcctgatggatcggattggcaggctcacgatgcttggggggtccatggtcctctccgggatctcgtgcttcttcctgtggttcggcacctcggagtccatgatgatcggaatgttgtgcctgtacaacggtctgaccatcagcgcctggaacagcctcgacgtggtcaccgtcgagctgtatcctaccgaccggcgcgcgacaggcttcggattcctgaacgcactgtgcaaggcagccgcggtcctgggaaatctgatctttggttcgctggtgtccatcactaagagcatccctattctgctcgcctccacggtgctcgtgtgtggtggcctggtcgggctgtgcctgcccgacactcgcacccaagtgctcatggactacaaggatgacgatgataagggagactacaaggacgatgacgacaagggggattacaaggacgacgatgacaaaggaagcggcgccactaacttttccctgctgaagcaggccggggacgtcgaagaaaaccccgggccaatgcgcaacattttcaagcggaatcaggagcctatcgtggccccggccaccactaccgccactatgcctattggacctgtcgacaactccacggaatcaggcggcgccggcgaatcccaagaggacatgttcgccaagctgaaggagaaactgttcaacgaaatcaacaagattcccctcccgccgtgggccctgatcgctatcgctgtcgtcgccggactgctgctgcttacttgctgcttctgcatttgcaagaagtgttgttgcaagaaaaagaaaaacaagaaggaaaaggggaagggaatgaagaacgccatgaatatgaaggacatgaagggcggacaggatgatgatgatgctgaaactgggctgactgagggcgaaggagagggcgaagaggagaaggaacctgagaacctgggaaagctccaattctccctggattacgacttccaagccaaccagctgactgtgggagtgttgcaagccgccgagctgccagccctggacatgggcggcacctccgacccctatgtgaaggtgttcttgctgcctgacaagaagaagaagtacgaaaccaaggtgcaccgcaagaccctgaaccccgctttcaacgaaaccttcactttcaaagtgccctaccaagagctcgggggaaagactctcgtgatggcgatctacgacttcgaccggttcagcaagcacgatatcatcggggaggtcaaggtcccgatgaacaccgtggaccttggccaaccgattgaagaatggcgcgatctccagggtggcgaaaaggaggagcccgagaaactgggtgacatctgtacatcactgcgctacgtgccgaccgccgggaagctcactgtctgcatcctggaggccaagaacctgaagaaaatggacgtgggcgggctctccgacccttacgtgaagatccacctgatgcagaacggaaagcggctgaagaagaagaaaaccactgtgaagaaaaagactctgaacccctacttcaacgagtcgttctccttcgaaatcccgtttgagcaaatccagaaggtccaagtggtcgtgactgtgcttgactacgacaagctcggaaagaacgaggccattggcaaaatcttcgtgggatcgaacgcaactggcaccgagctgagacactggtctgacatgctcgccaacccaaggcggccgattgctcagtggcactccttgaaacctgaggaagaagtggatgcccttcttggaaagaacaagatgtacccctacgacgtccctgattacgcgggatacccgtacgatgtgcctgactatgccggctacccgtacgatgtgccagactacgctggctccggagccacgaacttttcgctgctgaaacaggccggcgacgtggaagaaaatcccggtccaatgattgaacaagatggattgcacgctggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacatgcccattcgaccaccaagcgaaacatcgcatcgagcgagcacgtactcggatggaagccggtcttgtcgatcaggatgatctggacgaggaacatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaggaacttggcggcgaatgggctgaccgcttcctcgtgctttacggtatcgccgctcccgattcgcagcgcatcgccttctatcgccttcttgacgagttcttctgatag

SEQ ID NO: 5 - DNA, 5772 bp, Artificial Sequence; identical to SEQ ID NO: 4
  Synthetic

SEQ ID NO: 6 - protein, 356 aa, Artificial Sequence
  Synthetic
MSPCGRARRQTSRGAMAVLAWKFPRTRLPMGASALCVVVLCWLYIFPVYRLPNEKEIVQGVLQQGTAWRRNQTAARAFRKQMEDCCDPAHLFAMTKMNSPMGKSMWYDGEFLYSFTIDNSTYSLFPQATPFQLPLKKCAVVGNGGILKKSGCGRQIDEANFVMRCNLPPLSSEYTKDVGSKSQLVTANPSIIRQRFQNLLWSRKTFVDNMKIYNHSYIYMPAFSMKTGTEPSLRVYYTLSDVGANQTVLFANPNFLRSIGKFWKSRGIHAKRLSTGLFLVSAALGLCEEVAIYGFWPFSVNMHEQPISHHYYDNVLPFSGFHAMPEEFLQLWYLHKIGALRMQLDPCEDTSLQPTS

SEQ ID NO: 7 - protein, 22 aa, Artificial Sequence
  Synthetic
GSGATNFSLLKQAGDVEENPGP

SEQ ID NO: 8 - protein, 742 aa, Artificial Sequence
  Synthetic
MEEGFRDRAAFIRGAKDIAKEVKKHAAKKVVKGLDRVQDEYSRRSYSRFEEEDDDDDFPAPSDGYYRGEGTQDEEEGGASSDATEGHDEDDEIYEGEYQGIPRAESGGKGERMADGAPLAGVRGGLSDGEGPPGGRGEAQRRKEREELAQQYEAILRECGHGRFQWTLYFVLGLALMADGVEVFVVGFVLPSAEKDMCLSDSNKGMLGLIVYLGMMVGAFLWGGLADRLGRRQCLLISLSVNSVFAFFSSFVQGYGTFLFCRLLSGVGIGGSIPIVFSYFSEFLAQEKRGEHLSWLCMFWMIGGVYAAAMAWAIIPHYGWSFQMGSAYQFHSWRVFVLVCAFPSVFAIGALTTQPESPRFFLENGKHDEAWMVLKQVHDTNMRAKGHPERVFSVTHIKTIHQEDELIEIQSDTGTWYQRWGVRALSLGGQVWGNFLSCFGPEYRRITLMMMGVWFTMSFSYYGLTVWFPDMIRHLQAVDYASRTKVFPGERVEHVTFNFTLENQIHRGGQYFNDKFIGLRLKSVSFEDSLFEECYFEDVTSSNTFFRNCTFINTVFYNTDLFEYKFVNSRLINSTFLHNKEGCPLDVTGTGEGAYMVYFVSFLGTLAVLPGNIVSALLMDKIGRLRMLAGSSVMSCVSCFFLSFGNSESAMIALLCLFGGVSIASWNALDVLTVELYPSDKRTTAFGFLNALCKLAAVLGISIFTSFVGITKAAPILFASAALALGSSLALKLPETRGQVLQ

SEQ ID NO: 9 - protein, 727 aa, Artificial Sequence
  Synthetic
MEDSYKDRTSLMKGAKDIAREVKKQTVKKVNQAVDRAQDEYTQRSYSRFQDEEDDDDYYPAGETYNGEANDDEGSSEATEGHDEDDEIYEGEYQGIPSMNQAKDSIVSVGQPKGDEYKDRRELESERRADEEELAQQYELIIQECGHGRFQWALFFVLGMALMADGVEVFVVGFVLPSAETDLCIPNSGSGWLGSIVYLGMMVGAFFWGGLADKVGRKQSLLICMSVNGFFAFLSSFVQGYGFFLFCRLLSGFGIGGAIPTVFSYFAEVLAREKRGEHLSWLCMFWMIGGIYASAMAWAIIPHYGWSFSMGSAYQFHSWRVFVIVCALPCVSSVVALTFMPESPRFLLEVGKHDEAWMILKLIHDTNMRARGQPEKVFTVNKIKTPKQIDELIEIESDTGTWYRRCFVRIRTELYGIWLTFMRCFNYPVRDNTIKLTIVWFTLSFGYYGLSVWFPDVIKPLQSDEYALLTRNVERDKYANFTINFTMENQIHTGMEYDNGRFIGVKFKSVTFKDSVFKSCTFEDVTSVNTYFKNCTFIDTVFDNTDFEPYKFIDSEFKNCSFFHNKTGCQITFDDDYSAYWIYFVNFLGTLAVLPGNIVSALLMDRIGRLTMLGGSMVLSGISCFFLWFGTSESMMIGMLCLYNGLTISAWNSLDVVTVELYPTDRRATGFGFLNALCKAAAVLGNLIFGSLVSITKSIPILLASTVLVCGGLVGLCLPDTRTQVLM

SEQ ID NO: 10 - protein, 683 aa, Artificial Sequence
  Synthetic
MDDYKYQDNYGGYAPSDGYYRGNESNPEEDAQSDVTEGHDEEDEIYEGEYQGIPHPDDVKAKQAKMAPSRMDSLRGQTDLMAERLEDEEQLAHQYETIMDECGHGRFQWILFFVLGLALMADGVEVFVVSFALPSAEKDMCLSSSKKGMLGMIVYLGMMAGAFILGGLADKLGRKRVLSMSLAVNASFASLSSFVQGYGAFLFCRLISGIGIGGALPIVFAYFSEFLSREKRGEHLSWLGIFWMTGGLYASAMAWSIIPHYGWGFSMGTNYHFHSWRVFVIVCALPCTVSMVALKFMPESPRFLLEMGKHDEAWMILKQVHDTNMRAKGTPEKVFTVSNIKTPKQMDEFIEIQSSTGTWYQRWLVRFKTIFKQVWDNALYCVMGPYRMNTLILAVVWFAMAFSYYGLTVWFPDMIRYFQDEEYKSKMKVFFGEHVYGATINFTMENQIHQHGKLVNDKFTRMYFKHVLFEDTFFDECYFEDVTSTDTYFKNCTIESTIFYNTDLYEHKFINCRFINSTFLEQKEGCHMDLEQDNDFLIYLVSFLGSLSVLPGNIISALLMDRIGRLKMIGGSMLISAVCCFFLFFGNSESAMIGWQCLFCGTSIAAWNALDVITVELYPTNQRATAFGILNGLCKFGAILGNTIFASFVGITKVVPILLAAASLVGGGLIALRLPETREQVLM

SEQ ID NO: 11 - protein, 128 aa, Artificial Sequence
  Synthetic
FPDMIRHLQAVDYASRTKVFPGERVEHVTFNFTLENQIHRGGQYFNDKFIGLRLKSVSFEDSLFEECYFEDVTSSNTFFRNCTFINTVFYNTDLFEYKFVNSRLINSTFLHNKEGCPLDVTGTGEGAY

SEQ ID NO: 12 - protein, 130 aa, Artificial Sequence
  Synthetic
WFPDMIRYFQDEEYKSKMKVFFGEHVYGATINFTMENQIHQHGKLVNDKFTRMYFKHVLFEDTFFDECYFEDVTSTDTYFKNCTIESTIFYNTDLYEHKFINCRFINSTFLEQKEGCHMDLEQDNDFLIY

SEQ ID NO: 13 - protein, 128 aa, Artificial Sequence
  Synthetic
WFPDVIKPLQSDEYALLTRNVERDKYANFTINFTMENQIHTGMEYDNGRFIGVKFKSVTFKDSVFKSCTFEDVTSVNTYFKNCTFIDTVFDNTDFEPYKFIDSEFKNCSFFHNKTGCQITFDDDYSAY

SEQ ID NO: 14 - protein, 422 aa, Artificial Sequence
  Synthetic
MVSESHHEALAAPPVTTVATVLPSNATEPASPGEGKEDAFSKLKEKFMNELHKIPLPPWALIAIAIVAVLLVLTCCFCICKKCLFKKKNKKKGKEKGGKNAINMKDVKDLGKTMKDQALKDDDAETGLTDGEEKEEPKEEEKLGKLQYSLDYDFQNNQLLVGIIQAAELPALDMGGTSDPYVKVFLLPDKKKKFETKVHRKTLNPVFNEQFTFKVPYSELGGKTLVMAVYDFDRFSKHDIIGEFKVPMNTVDFGHVTEEWRDLQSAEKEEQEKLGDICFSLRYVPTAGKLTVVILEAKNLKKMDVGGLSDPYVKIHLMQNGKRLKKKKTTIKKNTLNPYYNESFSFEVPFEQIQKVQVVVTVLDYDKIGKNDAIGKVFVGYNSTGAELRHWSDMLANPRRPIAQWHTLQVEEEVDAMLAVKK

SEQ ID NO: 15 - protein, 419 aa, Artificial Sequence
  Synthetic
MRNIFKRNQEPIVAPATTTATMPIGPVDNSTESGGAGESQEDMFAKLKEKLFNEINKIPLPPWALIAIAVVAGLLLLTCCFCICKKCCCKKKKNKKEKGKGMKNAMNMKDMKGGQDDDDAETGLTEGEGEGEEEKEPENLGKLQFSLDYDFQANQLTVGVLQAAELPALDMGGTSDPYVKVFLLPDKKKKYETKVHRKTLNPAFNETFTFKVPYQELGGKTLVMAIYDFDRFSKHDIIGEVKVPMNTVDLGQPIEEWRDLQGGEKEEPEKLGDICTSLRYVPTAGKLTVCILEAKNLKKMDVGGLSDPYVKIHLMQNGKRLKKKKTTVKKKTLNPYFNESFSFEIPFEQIQKVQVVVTVLDYDKLGKNEAIGKIFVGSNATGTELRHWSDMLANPRRPIAQWHSLKPEEEVDALLGKNK

SEQ ID NO: 16 - protein, 232 aa, Artificial Sequence
  Synthetic
MVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYK

SEQ ID NO: 17 - protein, 248 aa, Artificial Sequence
  Synthetic
MASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTGFEDFVGDWRQTAGYNLDQVLEQGGVSSLFQ

SEQ ID NO: 18 - protein, 223 aa, Artificial Sequence
  Synthetic
LFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNYFSDNVYITADKQKNGIKANFKIRHNIEDGGVQLADHYQQNTPIGDGPVLLPDNHYLSTQSKLSKDPNEKRDHMVLLEFVTAAGL

SEQ ID NO: 19 - protein, 206 aa, Artificial Sequence
  Synthetic
MAEDADMRNELEEMQRRADQLADESLESTRRMLQLVEESKDAGIRTLVMLDEQGEQLERIEEGMDQINKDMKEAEKNLTDLGKFCGLCVCPCNKLKSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSG

SEQ ID NO: 20 - protein, 264 aa, Artificial Sequence [sequence omitted]

SEQ ID NO: 21 - protein, 199 aa, Artificial Sequence
  Synthetic
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 22 - protein, 169 aa, Artificial Sequence
  Synthetic
HTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA